(19) United States
(12) Patent Application Publication
(10) Pub. No.: US 2023/0140430 A1
(43) Pub. Date: May 4, 2023

(54) 1H-PYRAZOLO[4,3-d]PYRIMIDINE COMPOUNDS AS TOLL-LIKE RECEPTOR 7 (TLR7) AGONISTS

(71) Applicant: BRISTOL-MYERS SQUIBB COMPANY, Princeton, NJ (US)

(72) Inventors: Matthew COX, San Francisco, CA (US); Liqi HE, San Jose, CA (US); Sanjeev GANGWAR, Foster City, CA (US); Ashvinikumar V. GAVAI, Princeton Junction, NJ (US); Matthias BROEKEMA, New Hope, PA (US); Qiang CONG, Palo Alto, CA (US); Daniel O'MALLEY, New Hope, PA (US); Yam B. POUDEL, Fremont, CA (US); Christine M. TARBY, Lawrenceville, NJ (US)

(21) Appl. No.: 17/792,887

(22) PCT Filed: Jan. 26, 2021
(86) PCT No.: PCT/US2021/014976

§ 371 Date: Jul. 14, 2022

Related U.S. Application Data
(60) Provisional application No. 62/966,098, filed on Jan. 27, 2020.
(60) Provisional application No. 63/057,675, filed on Jul. 28, 2020.

(51) Int. Cl. C07D 519/00; A61K 45/06

(52) U.S. Cl. CPC C07D 519/00; A61K 45/06

(57) ABSTRACT

Compounds according to formula I are useful as agonists of Toll-like receptor 7 (TLR7). Such compounds can be used in cancer treatment, especially in combination with an anti-cancer immunotherapy agent, or as a vaccine adjuvant.

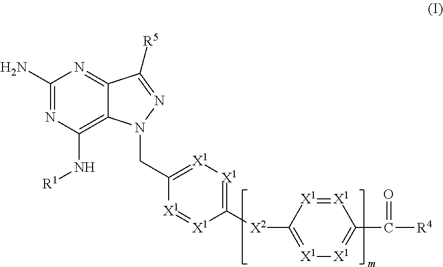

### CROSS-REFERENCE TO RELATED APPLICATIONS

This application claims the benefit under 35 U.S.C. § 119(e) of U.S. Provisional Application Ser. No. 63/057,675, filed Jul. 28, 2020, and US Provisional Application Ser. No. 62/966,098, filed Jan. 27, 2020; the disclosures of which are incorporated herein by reference.

### BACKGROUND OF THE DISCLOSURE

This disclosure relates to Toll-like receptor 7 (“TLR7”) agonists and conjugates thereof, and methods for the preparation and use of such agonists and their conjugates.

Toll-like receptors (“TLRs”) are receptors that recognize pathogen-associated molecular patterns (“PAMPs”), which are small molecular motifs conserved in certain classes of pathogens. TLRs can be located either on a cell's surface or intracellularly. Activation of a TLR by the binding of its cognate PAMP signals the presence of the associated pathogen inside the host—i.e., an infection—and stimulates the host's immune system to fight the infection. Humans have 10 TLRs, named TLR1, TLR2, TLR3, and so on.

The activation of a TLR—with TLR7 being the most studied—by an agonist can have a positive effect on the action of vaccines and immunotherapy agents in treating a variety of conditions other than actual pathogen infection, by stimulating the immune response overall. Thus, there is considerable interest in the use of TLR7 agonists as vaccine adjuvants or as enhancers in cancer immunotherapy. See, for example, Vasilakos and Tomai 2013, Sato-Kaneko et al. 2017, Smits et al. 2008, and Ota et al. 2019.

TLR7, an intracellular receptor located on the membrane of endosomes, recognizes PAMPs associated with single-stranded RNA viruses. Its activation induces secretion of Type I interferons such as IFNα and IFNβ (Lund et al. 2004). TLR7 has two binding sites, one for single stranded RNA ligands (Berghöfer et al. 2007) and one for small molecules such as guanosine (Zhang et al. 2016).

TLR7 can bind to, and be activated by, guanosine-like synthetic agonists such as imiquimod, resiquimod, and gardiquimod, which are based on a 1H-imidazo[4,5-c]quinoline scaffold. For a review of small-molecule TLR7 agonists, see Cortez and Va 2018.

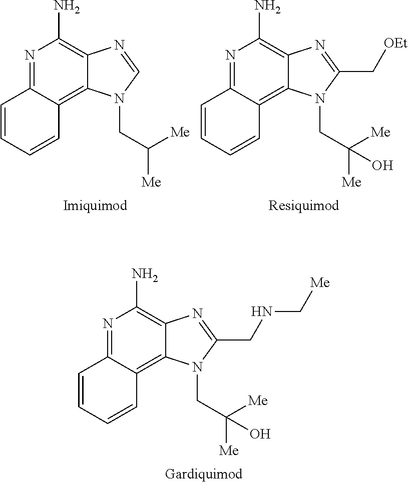

Synthetic TLR7 agonists based on a pteridinone molecular scaffold are also known, as exemplified by vesatolimod (Desai et al. 2015).

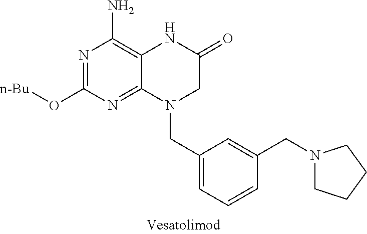

Other synthetic TLR7 agonists based on a purine-like scaffold have been disclosed, frequently according to the general formula (A):

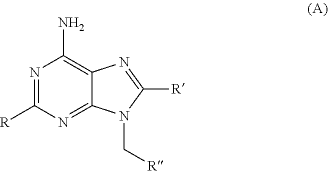

where R, R′, and R″ are structural variables, with R″ typically containing an unsubstituted or substituted aromatic or heteroaromatic ring.

Disclosures of bioactive molecules having a purine-like scaffold and their uses in treating conditions such as fibrosis, inflammatory disorders, cancer, or pathogenic infections include: Akinbobuyi et al. 2015 and 2016; Barberis et al. 2012; Carson et al. 2014; Ding et al. 2016, 2017a, and 2017b; Graupe et al. 2015; Hashimoto et al. 2009; He et al. 2019a and 2019b; Holldack et al. 2012; Isobe et al. 2009a and 2012; Poudel et al. 2019a and 2019b; Pryde 2010; and Young et al. 2019.

The group R″ can be pyridyl: Bonfanti et al. 2015a and 2015b; Halcomb et al. 2015; Hirota et al. 2000; Isobe et al. 2002, 2004, 2006, 2009a, 2009b, 2011, and 2012; Kasibhatla et al. 2007; Koga-Yamakawa et al. 2013; Musmuca et al. 2009; Nakamura 2012; Ogita et al. 2007; and Yu et al. 2013.

There are disclosures of related molecules in which the 6,5-fused ring system of formula (A)—a pyrimidine six member ring fused to an imidazole five member ring—is modified. (a) Dellaria et al. 2007, Jones et al. 2010 and 2012, and Pilatte et al. 2017 disclose compounds in which the pyrimidine ring is replaced by a pyridine ring. (b) Chen et al. 2011, Coe et al. 2017, Poudel et al. 2020a and 2020b, and Zhang et al. 2018 disclose compounds in which the imidazole ring is replaced by a pyrazole ring. (c) Cortez et al. 2017 and 2018; Li et al. 2018; and McGowan et al. 2016a, 2016b, and 2017 disclose compounds in which the imidazole ring is replaced by a pyrrole ring.

Bonfanti et al. 2015b and 2016 and Purandare et al. 2019 disclose TLR7 modulators in which the two rings of a purine moiety are spanned by a macrocycle:

A TLR7 agonist can be conjugated to a partner molecule, which can be, for example, a phospholipid, a poly(ethylene glycol) (“PEG”), an antibody, or another TLR (commonly TLR2). Exemplary disclosures include: Carson et al. 2013, 2015, and 2016, Chan et al. 2009 and 2011, Cortez et al. 2017, Gadd et al. 2015, Lioux et al. 2016, Maj et al. 2015, Vernejoul et al. 2014, and Zurawski et al. 2012. A frequent conjugation site is at the R″ group of formula (A).

Jensen et al. 2015 discloses the use of cationic lipid vehicles for the delivery of TLR7 agonists.

Some TLR7 agonists, including resiquimod are dual TLR7/TLR8 agonists. See, for example, Beesu et al. 2017, Embrechts et al. 2018, Lioux et al. 2016, and Vernejoul et al. 2014.

Full citations for the documents cited herein by first author or inventor and year are listed at the end of this specification.

### BRIEF SUMMARY OF THE DISCLOSURE

This specification relates to compounds having a 1H-pyrazolo[4,3d]pyrimidine aromatic system, having activity as TLR7 agonists.

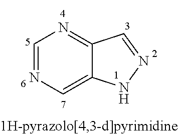

In one aspect, there is provided a compound with a structure according to formula I

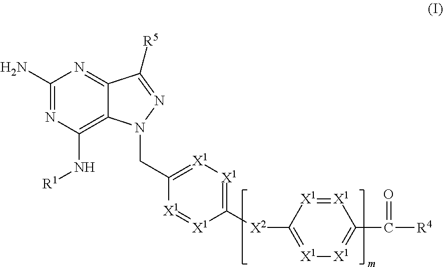

wherein
each X¹ is independently N or CR²;
X² is O, CH₂, NH, S, or N(C₁-C₃ alkyl);
R¹ is (C₁-C₅ alkyl),
   (C₂-C₅ alkenyl),
   (C₁-C₈ alkanediyl)₀₋₁(C₃-C₆ cycloalkyl),
   (C₂-C₈ alkanediyl)OH,
   (C₂-C₈ alkanediyl)O(C₁-C₃ alkyl),
   (C₁-C₄ alkanediyl)₀₋₁(5-6 membered heteroaryl),
   (C₁-C₄ alkanediyl)₀₋₁phenyl,
   (C₁-C₄ alkanediyl)CF₃,
   (C₂-C₈ alkanediyl)N[C(═O)](C₁-C₃ alkyl),
   or
   (C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R⁴ is NH₂,
   NH(C₁-C₅ alkyl),
   N(C₁-C₅ alkyl)₂,
   NH(C₁-C₄ alkanediyl)₀₋₁(C₃-C₅ cycloalkyl),
   N(C₃-C₆ cycloalkyl)₂,
   or
   a moiety having the structure

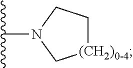

R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl,

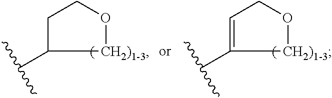

R^{x} and R^{y} are independently H or C₁-C₃ alkyl or R^{x} and R^{y} combine with the nitrogen to which they are bonded to form a 3- to 7-membered heterocycle;
and
m is 0 or 1;
wherein in R¹, R², R⁴, and R⁵
   an alkyl, alkanediyl, cycloalkyl, phenyl, 5 or 6-membered heteroaryl, or moiety of the formula

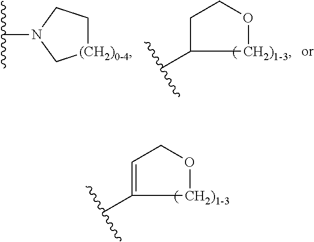

is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl);
and
an alkyl, alkanediyl, cycloalkyl, or cyclic moiety of the formula

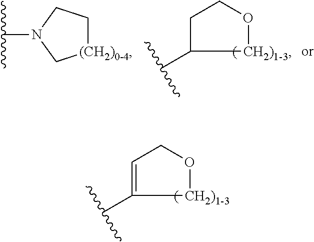

may have a CH₂ group replaced by O, SO₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

Compounds disclosed herein have activity as TLR7 agonists and some can be conjugated to an antibody for targeted delivery to a target tissue or organ of intended action. They can also be PEGylated, to modulate their pharmaceutical properties.

Compounds disclosed herein, or their conjugates or their PEGylated derivatives, can be used in the treatment of a subject suffering from a condition amenable to treatment by activation of the immune system, by administering to such subject a therapeutically effective amount of such a compound or a conjugate thereof or a PEGylated derivative thereof, especially in combination with a vaccine or a cancer immunotherapy agent.

### DETAILED DESCRIPTION OF THE DISCLOSURE

### Compounds

In one aspect, in formula (I), the moiety

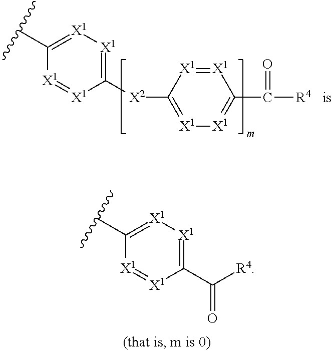

In one aspect, compounds of this disclosure are according to formula (Ia), wherein R¹, R², R⁴, and R⁵ are as defined in respect of formula (I):

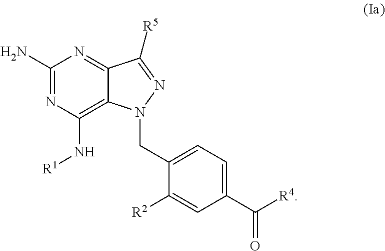

In another aspect, compounds of this disclosure are according to formula (Ib), wherein R¹, R⁴, and R⁵ are as defined in respect of formula (I):

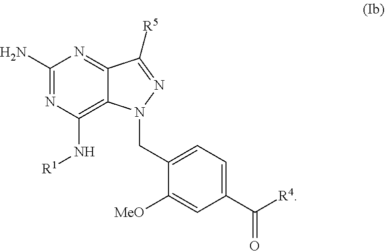

In formula (Ib), R⁵ preferably is H.

In another aspect, this disclosure provides a compound having a structure according to formula (Ib) wherein
R¹ is

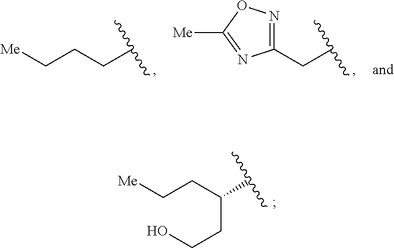 [SMILES: *CCCC ; *Cc1noc(C)n1 ; *[C@@H](CCC)CCO]

R⁴ is

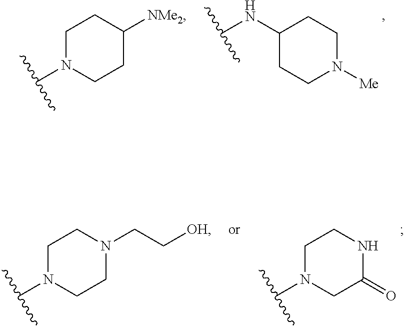 [SMILES: *N1CCC(N(C)C)CC1 ; *NC1CCN(C)CC1 ; *N1CCN(CCO)CC1 ; *N1CCNC(=O)C1]

and
R⁵ is H, Me, or F.

Preferably, R¹ is selected from the following group (“preferred R¹ group”), consisting of

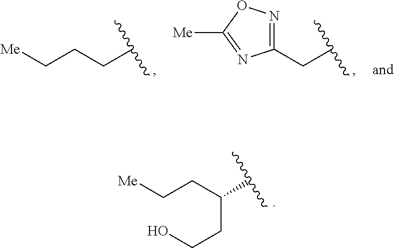 [SMILES: *CCCC ; *Cc1noc(C)n1 ; *[C@@H](CCC)CCO]

In another embodiment, R¹ is

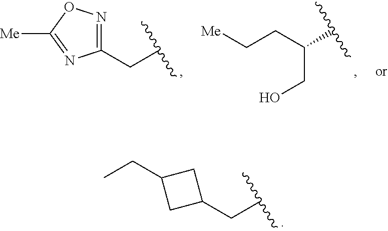

Examples of groups R² include (with the first being preferred)

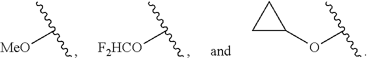

Examples of groups R⁴ include:

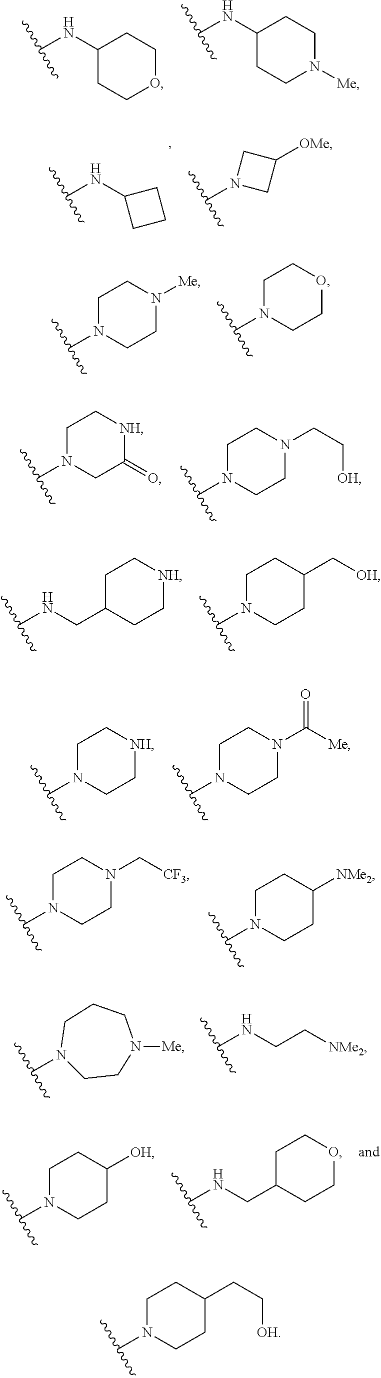

Preferably, R⁴ is selected from the following group (“preferred R⁴ group”), consisting of:

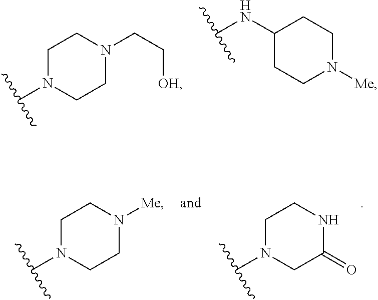 [SMILES: *N1CCN(CCO)CC1 ; *NC1CCN(C)CC1 ; *N1CCN(C)CC1 ; *N1CCNC(=O)C1]

Examples of suitable groups, R⁵ are H,

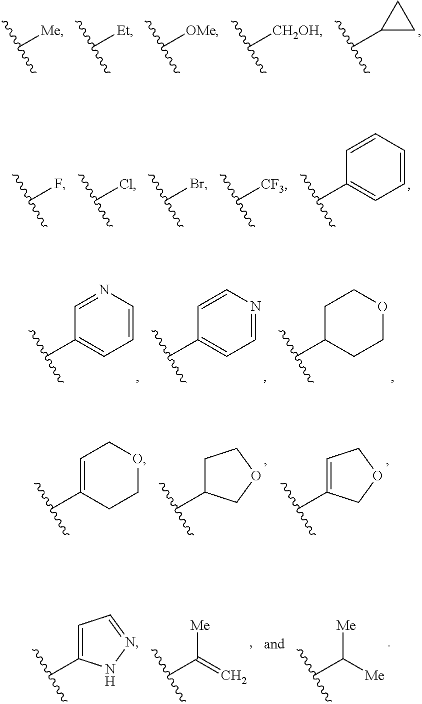

Preferably, R⁵ is H or Me.

Preferably, R⁴ is selected from the preferred R⁴ group, in combination with an R³ selected from the preferred R³ group; an R¹ selected from the preferred R¹ group, and R⁵ equals H or Me.

By way of exemplification and not of limitation, moieties of the formula

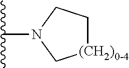 [SMILES: *N1CCCC1]

include

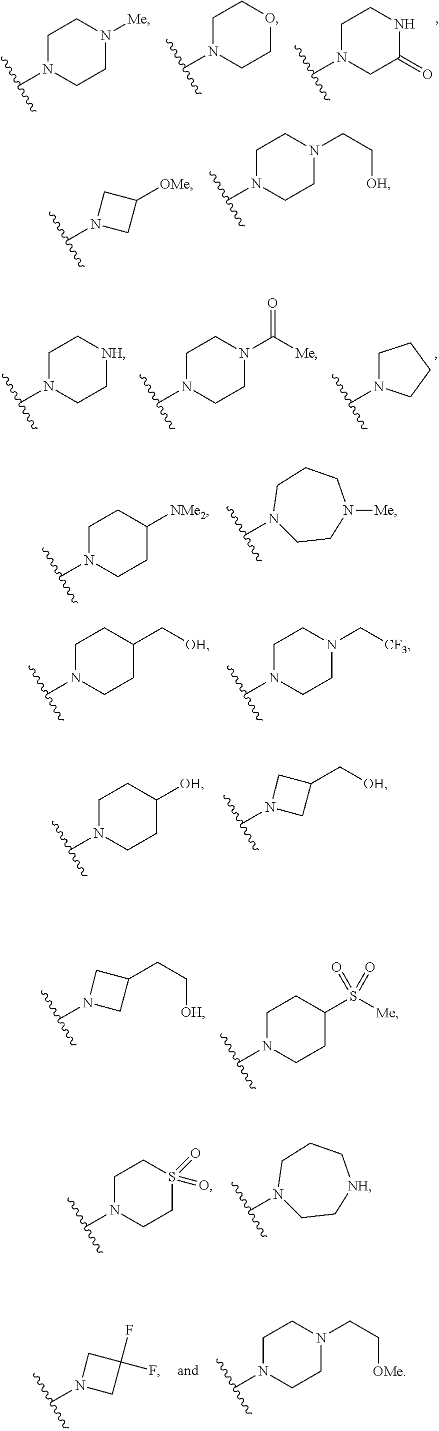

Some of the above exemplary moieties of the formula

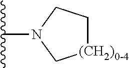 [SMILES: *N1CCCC1]

bear optional substituents and/or optionally have one or more CH₂ groups replaced by O, SO₂, etc., as described in the BRIEF SUMMARY OF THE DISCLOSURE above.

Specific examples of compounds disclosed herein are shown in the following Table A. The table also provides data relating to biological activity: human TLR7 agonism reporter assay and/or induction of the CD69 gene in human whole blood, determined per the procedures provided hereinbelow. The right-most column contains analytical data (mass spectrum, LC/MS retention time, and NMR). In one embodiment, a compound of this disclosure has (a) a human TLR7 (hTLR7) Reporter Assay EC₅₀ value of less than 1,000 nM and (b) a human whole blood (hWB) CD69 induction EC₅₀ value of less than 1,000 nM. (Where an assay was performed multiple times, the reported value is an average.)

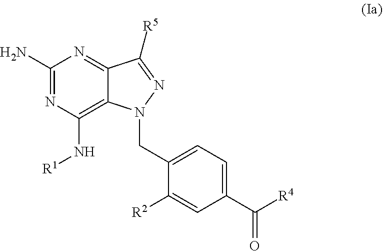

TABLE A
Compounds According to Formula (Ia)

| | | hTLR7 | hWB | Analytical Data |
|---|---|---|---|---|
| Cpd. | Structure | Agonism | CD69 | (LC/MS, Liquid Chromatography RT, |
| No. | (R⁵ = H unless noted otherwise) | EC₅₀ (nM) | EC₅₀ (nM) | ¹H NMR (500 MHz, DMSO-d₆)) |
| 101 | 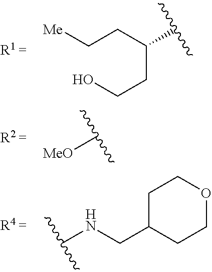 | 1,296.4 | 1000 | [M + H]⁺ 511.2 RT (min)/Method 1.05/I δ 8.50 (d, J = 6.0 Hz, 1H), 7.60 (s, 1H), 7.49 (d, J = 1.6 Hz, 1H), 7.31 (d, J = 8.2 Hz, 1H), 6.42 (d, J = 7.9 Hz, 1H), 5.77 (d, J = 17.3 Hz, 1H), 5.67- 5.59 (m, 3H), 4.33 (s, 3H), 3.35 (s, 1H), 3.17 (dt, J = 12.1, 6.2 Hz, 4H), 2.73 (t, J = 12.6 Hz, 2H), 1.74 (d, J = 13.7 Hz, 3H), 1.68-1.61 (m, 1H), 1.56 (d, J = 5.5 Hz, 1H), 1.45-1.23 (m, 5H), 1.01 (q, J = 7.7 Hz, 2H), 0.73 (t, J = 7.3 Hz, 3H). |
| | 4-[(5-amino-7-{[(3S)-1-hydroxy- | | | |
| | hexan-3-yl]amino}-1H-pyrazolo[4,3- | | | |
| | d]pyrimidin-1-yl)methyl]-3-methoxy- | | | |
| | N-[(piperidin-4-yl)methyl]benzamide | | | |
| 102 | 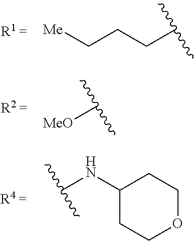 | 644.4 | 1000 | [M + H]⁺ 454.0 RT (min)/Method 1.09/A δ 8.27 (br d, J = 7.6 Hz, 1H), 7.58 (s, 1H), 7.45 (s, 1H), 7.30 (d, J = 8.2 Hz, 1H), 6.52 (br s, 1H), 6.49 (d, J = 7.6 Hz, 1H), 5.68 (s, 2H), 5.66 (s, 2H), 4.04-3.92 (m, 1H), 3.92-3.85 (m, 5H), 3.43-3.32 (m, 2H), 1.74 (br d, J = 12.5 Hz, 2H), 1.56 (qd, J = 11.9, 4.3 Hz, 2H), 1.47 (quin, J = 7.3 Hz, 2H), 1.24-1.08 (m, 2H), 0.82 (t, J = 7.3 Hz, 3H) |
| | 4-{[5-amino-7-(butylamino)-1H- | | | |
| | pyrazolo[4,3-d]pyrimidin-1- | | | |
| | yl]methyl}-3-methoxy-N-(oxan-4- | | | |
| | yl)benzamide | | | |
| 103 | 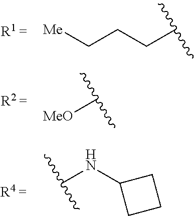 | 1,658.4 | 1000 | [M + H]⁺ 424.1 RT (min)/Method 1.41/A δ 8.61 (br d, J = 7.7 Hz, 1H), 8.26 (br s, 1H), 7.82 (br s, 1H), 7.77 (s, 1H), 7.44 (s, 1H), 7.37 (d, J = 7.8 Hz, 1H), 6.84 (d, J = 7.9 Hz, 1H), 5.77 (s, 2H), 4.47-4.34 (m, 1H), 3.83 (s, 3H), 3.64-3.50 (m, 2H), 2.26-2.17 (m, 2H), 2.11-2.01 (m, 2H), 1.74-1.62 (m, 2H), 1.57 (quin, J = 7.4 Hz, 2H), 1.24 (dq, J = 15.0, 7.4 Hz, 2H), 0.87 (t, J = 7.3 Hz, 3H) |
| | 4-{[5-amino-7-(butylamino)-1H- | | | |
| | pyrazolo[4,3-d]pyrimidin-1- | | | |
| | yl]methyl}-N-cyclobutyl-3- | | | |
| | methoxybenzamide | | | |
| 104 | 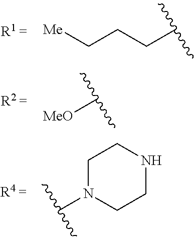 | 40.7 | 119.8 | [M + H]⁺ 439.2 RT (min)/Method 0.78/A δ 7.59 (s, 1H), 7.00 (s, 1H), 6.81 (d, J = 7.6 Hz, 1H), 6.53 (br s, 1H), 6.44 (d,J = 7.6 Hz, 1H), 5.66 (s, 2H), 5.64 (s, 2H), 3.86 (s, 3H), 3.43-3.35 (m, 2H), 2.80-2.57 (m, 4H), 1.47 (quin, J = 7.2 Hz, 2H), 1.22-1.12 (m, 2H), 0.82 (t, J = 7.5 Hz, 3H) |
| | N7-butyl-1-{[2-methoxy-4-(pipera- | | | |
| | zine-1-carbonyl)phenyl]methyl}-1H- | | | |
| | pyrazolo[4,3-d]pyrimidine-5,7- | | | |
| | diamine | | | |
| 105 | 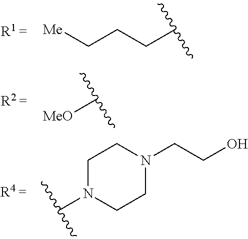 | 32.6 | 32.1 | [M + H]⁺ 483.2 RT (min)/Method 0.95/A δ 7.60 (s, 1H), 7.02 (s, 1H), 6.83 (d, J = 7.6 Hz, 1H), 6.62-6.51 (m, 1H), 6.45 (d, J = 7.9 Hz, 1H), 5.72-5.64 (m, 4H), 3.87 (s, 3H), 3.45-3.36 (m, 1H), 2.50-2.31 (m, 6H), 1.48 (quin, J = 7.2 Hz, 2H), 1.19 (sxt, J = 7.4 Hz, 2H), 0.84 (t, J = 7.5 Hz, 3H) |
| | 2-[4-(4-{[5-amino-7-(butylamino)-1H- | | | |
| | pyrazolo[4,3-d]pyrimidin-1- | | | |
| | yl]methyl}-3-methoxybenzoyl)- | | | |
| | piperazin-1-yl]ethan-1-ol | | | |
| 106 | 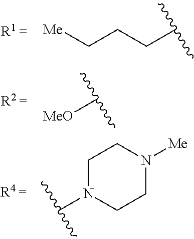 | 20.8 | 4.5 | [M + H]⁺ 453.1 RT (min)/Method 1.01/A δ 7.62 (s, 1H), 7.03 (s, 1H), 6.92- 6.75 (m, 2H), 6.49 (d, J = 7.6 Hz, 1H), 6.00 (br s, 2H), 5.69 (s, 2H), 3.87 (s, 3H), 3.10-3.04 (m, 1H), 2.44-2.26 (m, 4H), 2.22 (s, 3H), 1.50 (quin, J = 7.2 Hz, 2H), 1.23-1.15 (m, 2H), 0.85 (t, J = 7.3 Hz, 3H) |
| | N7-butyl-1-{[2-methoxy-4-(4- | | | |
| | methylpiperazine-1-carbonyl)- | | | |
| | phenyl]methyl}-1H-pyrazolo[4,3- | | | |
| | d]pyrimidine-5,7-diamine | | | |
| 107 | 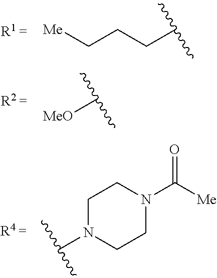 | 264.4 | 93.4 | [M + H]⁺ 481.2 RT (min)/Method 0.99/A δ 7.59 (s, 1H), 7.06 (s, 1H), 6.86 (d, J = 7.6 Hz, 1H), 6.62 (br s, 1H), 6.47 (br d, J = 7.6 Hz, 1H), 5.72 (s, 2H), 5.67 (s, 2H), 3.46-3.38 (m, 1H), 2.01 (brs, 3H), 1.92 (s, 2H), 1.56- 1.40 (m, 2H), 1.24-1.14 (m, 2H), 0.83 (t, J = 7.3 Hz, 3H) |
| | 1-[4-(4-{[5-amino-7-(butylamino)-1H- | | | |
| | pyrazolo[4,3-d]pyrimidin-1-yl]methyl}- | | | |
| | 3-methoxybenzoyl)piperazin-1- | | | |
| | yl]ethan-1-one | | | |
| 108 | 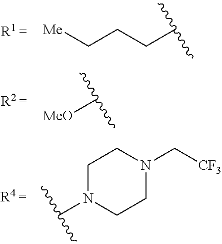 | 602.3 | 1000 | [M + H]⁺ 521.2 RT (min)/Method 1.39/A δ 7.59 (s, 1H), 7.03 (s, 1H), 6.83 (d, J = 7.6 Hz, 1H), 6.54 (br s, 1H), 6.45 (d, J = 7.9 Hz, 1H), 5.67 (s, 2H), 5.65 (s, 2H), 3.87 (s, 3H), 3.40 (br d, J = 6.1 Hz, 1H), 3.29-3.09 (m, 3H), 2.78-2.57 (m, 4H), 1.52-1.43 (m, 2H), 1.23-1.13 (m, 2H), 0.83 (t, J = 7.5 Hz, 3H) |
| | N7-butyl-1-({2-methoxy-4-[4-(2,2,2- | | | |
| | trifluoroethyl)piperazine-1-carbonyl] | | | |
| | phenyl}methyl)-1H-pyrazolo[4,3- | | | |
| | d]pyrimidine-5,7-diamine | | | |
| 109 | 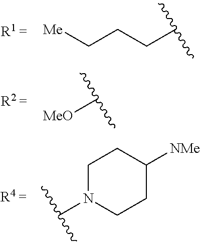 | 29.7 | 49.6 | δ[M + H]⁺ 480.9 RT (min)/Method 1.19/A δ 7.59 (s, 1H), 7.02 (s, 1H), 6.83 (d, J = 7.9 Hz, 1H), 6.57 (br s, 1H), 6.43 (d, J = 7.6 Hz, 1H), 5.69 (br s, 2H), 5.67 (s, 2H), 3.87 (s, 3H), 3.18 (br s, 1H), 3.09-2.70 (m, 3H), 2.31 (br s, 4H), 2.26 (br d, J = 6.1 Hz, 1H), 1.72 (br s, 3H), 1.48 (quin, J = 7.2 Hz, 2H), 1.36 (br s, 2H), 1.23-1.13 (m, 2H), 0.84 (t, J = 7.3 Hz, 3H) |
| | N7-butyl-1-({4-[4-(dimethylamino)- | | | |
| | piperidine-1-carbonyl]-2-methoxy- | | | |
| | phenyl}methyl)-1H-pyrazolo[4,3- | | | |
| | d]pyrimidine-5,7-diamine | | | |
| 110 | 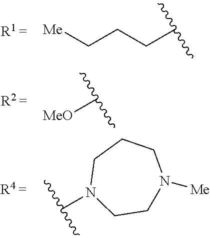 | 16.3 | 3.9 | [M + H]⁺ 467.1 RT (min)/Method 1.23/A δ 7.59 (s, 1H), 7.01 (s, 1H), 6.82 (br d, J = 7.3 Hz, 1H), 6.53 (br s, 1H), 6.43 (d, J = 7.9 Hz, 1H), 5.67 (s, 4H), 3.87 (s, 3H), 2.65 (br s, 1H), 2.30 (s, 2H), 2.24 (s, 1H), 1.84 (br s, 1H), 1.78-1.67 (m, 1H), 1.59-1.42 (m, 2H), 1.20 (br s, 2H), 0.88-0.80 (m, 3H) |
| | N7-butyl-1-{[2-methoxy-4-(4-methyl- | | | |
| | 1,4-diazepane-1-carbonyl)phenyl]- | | | |
| | methyl}-1H-pyrazolo[4,3- | | | |
| | d]pyrimidine-5,7-diamine | | | |
| 111 | 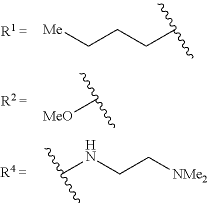 | 78.0 | 6.8 | [M + H]⁺ 441.1 RT (min)/Method 1.11/A δ 8.43-8.36 (m, 1H), 7.58 (s, 1H), 7.46 (s, 1H), 7.29 (d, J = 7.9 Hz, 1H), 6.52 (br t, J = 5.3 Hz, 1H), 6.48 (d, J = 7.6 Hz, 1H), 5.68 (s, 2H), 5.66 (s, 2H), 3.89 (s, 3H), 3.43-3.31 (m, 1H), 2.45 (br t, J = 6.7 Hz, 2H), 2.22 (s, 6H), 1.47 (quin, J = 7.2 Hz, 2H), 1.21-1.12 (m, 2H), 0.82 (t, J = 7.3 Hz, 3H) |
| | 4-{[5-amino-7-(butylamino)-1H- | | | |
| | pyrazolo[4,3-d]pyrimidin-1-yl] | | | |
| | methyl}-N-[2-(dimethylamino) | | | |
| | ethyl]-3-methoxybenzamide | | | |
| 112 | 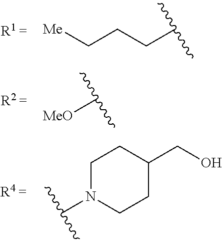 | 153.6 | 81.9 | [M + H]⁺ 468.2 RT (min)/Method 1.3/A δ 7.60 (s, 1H), 6.99 (s, 1H), 6.80 (d, J = 7.7 Hz, 1H), 6.64 (br s, 1H), 6.44 (d, J = 7.7 Hz, 1H), 5.79 (br s, 2H), 5.67 (s, 2H), 4.63-4.53 (m, 1H), 3.85 (s, 3H), 3.47-3.33 (m, 1H), 3.25 (br s, 1H), 2.98 (br s, 1H), 2.70 (br d, J = 12.9 Hz, 1H), 1.71 (br s, 1H), 1.62 (br s, 2H), 1.47 (quin, J = 7.2 Hz, 2H), 1.24-1.10 (m, 2H), 1.05 (br s, 2H), 0.82 (t, J = 7.4 Hz, 3H) |
| | [1-(4-{[5-amino-7-(butylamino)-1H- | | | |
| | pyrazolo[4,3-d]pyrimidin-1- | | | |
| | yl]methyl}-3-methoxybenzoyl)- | | | |
| | piperidin-4-yl]methanol | | | |
| 113 | 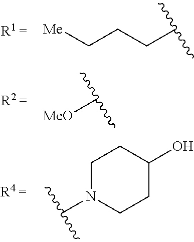 | 110.7 | 105.5 | [M + H]⁺ 454.2 RT (min)/Method 1.18/A δ 8.28 (br t, J = 5.6 Hz, 1H), 7.86 (br s, 1H), 7.79 (s, 1H), 7.01 (s, 1H), 6.87 (d, J = 7.6 Hz, 1H), 6.80 (d, J = 7.6 Hz, 1H), 5.76 (s, 2H), 3.82-3.78 (s, 3H), 3.78-3.68 (m, 1H), 3.66-3.53 (m, 1H), 3.36-3.02 (m, 2H), 1.92- 1.62 (m, 2H), 1.57 (quin, J = 7.2 Hz, 2H), 1.38 (br s, 1H), 1.30-1.19 (m, 2H), 0.88 (t, J = 7.3 Hz, 3H) |
| | 1-(4-{[5-amino-7-(butylamino)-1H- | | | |
| | pyrazolo[4,3-d]pyrimidin-1- | | | |
| | yl]methyl}-3-methoxybenzoyl)- | | | |
| | piperidin-4-ol | | | |
| 114 | 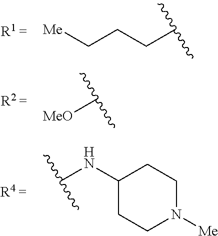 | 68.9 | 24.4 | [M + H]⁺ 467.2 RT (min)/Method 1.01/A δ 8.21 (br d, J = 7.9 Hz, 1H), 7.58 (s, 1H), 7.44 (s, 1H), 7.30 (d, J = 7.3 Hz, 1H), 6.53-6.49 (m, 1H), 6.48 (d, J = 7.9 Hz, 1H), 5.68 (s, 2H), 5.65 (s, 2H), 3.90 (s, 3H), 3.73 (br d, J = 7.9 Hz, 1H), 3.42-3.34 (m, 1H), 2.81 (br d, J = 11.6 Hz, 2H), 2.21 (s, 3H), 2.12-1.97 (m, 2H), 1.76 (br d, J = 11.0 Hz, 2H), 1.66-1.53 (m, 2H), 1.47 (quin, J = 7.3 Hz, 2H), 1.21- 1.12 (m, 2H), 0.82 (t, J = 7.3 Hz, 3H) |
| | 4-{[5-amino-7-(butylamino)-1H- | | | |
| | pyrazolo[4,3-d]pyrimidin-1- | | | |
| | yl]methyl}-3-methoxy-N-(1- | | | |
| | methylpiperidin-4-yl)benzamide | | | |
| 115 | 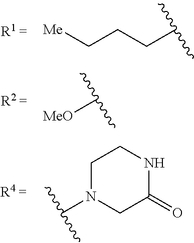 | 383.8 | 223.7 | [M + H]⁺ 453.2 RT (min)/Method 1.13/A δ 8.11 (br s, 1H), 7.60 (s, 1H), 7.10 (s, 1H), 6.90 (d, J = 7.9 Hz, 1H), 6.59 (br s, 1H), 6.45 (d, J = 7.9 Hz, 1H), 5.72 (s, 2H), 5.69 (s, 2H), 4.11-4.01 (m, 1H), 3.88 (s, 3H), 3.21 (br s, 1H), 1.49 (quin, J = 7.2 Hz, 2H), 1.27- 1.13 (m, 2H), 0.84 (t, J = 7.5 Hz, 3H) |
| | 4-(4-{[5-amino-7-(butylamino)-1H- | | | |
| | pyrazolo[4,3-d]pyrimidin-1- | | | |
| | yl]methyl}-3-methoxybenzoyl)- | | | |
| | piperazin-2-one | | | |
| 116 | 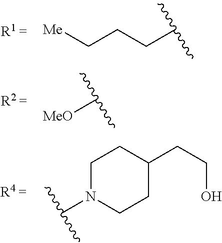 | 41.6 | 42.6 | [M + H]⁺ 482.2 RT (min)/Method 1.29/A δ 7.67 (s, 1H), 7.27-7.16 (m, 1H), 7.00 (s, 1H), 6.82 (d, J = 7.9 Hz, 1H), 6.58 (d, J = 7.6 Hz, 1H), 5.70 (s, 2H), 3.83 (s, 3H), 3.49-3.37 (m, 1H), 2.97 (br s, 1H), 2.71 (br d, J = 15.6 Hz, 1H), 1.83-1.56 (m, 3H), 1.51 (dt, J = 14.6, 7.3 Hz, 2H), 1.37 (br d, J = 5.8 Hz, 2H), 1.21 (sxt, J = 7.4 Hz, 2H), 1.06 (br s, 2H), 0.85 (t, J = 7.3 Hz, 3H) |
| | 2-[1-(4-{[5-amino-7-(butylamino)-1H- | | | |
| | pyrazolo[4,3-d]pyrimidin-1- | | | |
| | yl]methyl}-3-methoxybenzoyl)- | | | |
| | piperidin-4-yl]ethan-1-ol | | | |
| 117 | 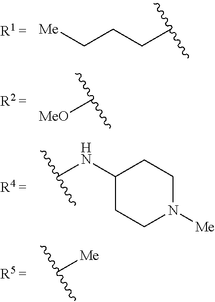 | 467.5 | 56.9 | [M + H]⁺ 481.1 RT (min)/Method 1.34/E δ 8.23 (br d, J = 7.6 Hz, 1H), 7.42 (s, 1H), 7.28 (d, J = 7.9 Hz, 1H), 6.47 (br d,J = 7.9 Hz, 1H), 5.63 (s, 1H), 5.59 (s, 2H), 3.88 (s, 3H), 3.80-3.68 (m, 1H), 3.64-3.50 (m, 1H), 3.38 (br d, J = 6.1 Hz, 1H), 2.82 (br d, J = 11.9 Hz, 2H), 2.24 (s, 2H), 2.21 (s, 3H), 2.06 (br t, J = 11.3 Hz, 2H), 1.91 (s, 3H), 1.76 (br d, J = 11.6 Hz, 2H), 1.67- 1.52 (m, 2H), 1.46 (quin, J = 7.3 Hz, 2H), 1.29-1.10 (m, 2H), 0.82 (t, J = 7.5 Hz, 3H) |
| | 4-{[5-amino-7-(butylamino)-3- | | | |
| | methyl-1H-pyrazolo[4,3-d]pyrimidin- | | | |
| | 1-yl]methyl}-3-methoxy-N-(1- | | | |
| | methylpiperidin-4-yl)benzamide | | | |
| 118 | 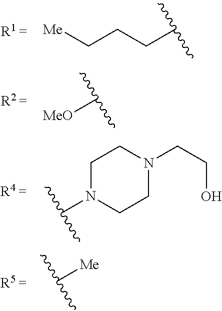 | 405.5 | 63.3 | [M + H]⁺ 497.3 RT (min)/Method 0.8/E δ 7.00 (s, 1H), 6.82 (d, J = 7.6 Hz, 1H), 6.53 (br s, 1H), 6.43 (d, J = 7.6 Hz, 1H), 5.69 (br s, 1H), 5.59 (s, 2H), 3.86 (s, 3H), 3.44-3.36 (m, 2H), 2.49-2.3 (m, 6H), 2.25 (s, 3H), 1.92 (s, 2H), 1.47 (quin, J = 7.2 Hz, 2H), 1.18 (dq, J = 14.8, 7.3 Hz, 2H), 0.83 (t, J = 7.3 Hz, 3H) |
| | 2-[4-(4-{[5-amino-7-(butylamino)-3- | | | |
| | methyl-1H-pyrazolo[4,3-d]pyrimidin- | | | |
| | 1-yl]methyl}-3-methoxybenzoyl)- | | | |
| | piperazin-1-yl]ethan-1-ol | | | |
| 119 | 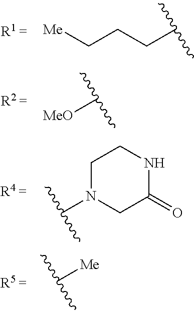 | 967.8 | 332.4 | [M + H]⁺ 467.2 RT (min)/Method 1.21/E δ 8.21 (br s, 1H), 8.13 (br s, 1H), 7.06 (s, 1H), 6.94 (d, J = 7.3 Hz, 1H), 6.82 (br d, J = 7.6 Hz, 1H), 5.67 (s, 2H), 4.13-4.01 (m, 2H), 3.79 (s, 3H), 3.69 (br d, J = 10.7 Hz, 2H), 3.57 (br d, J = 6.4 Hz, 2H), 3.22 (br s, 2H), 2.30 (s, 3H), 1.55 (quin, J = 7.3 Hz, 2H), 1.31-1.15 (m, 2H), 0.86 (t, J = 7.3 Hz, 3H) |
| | 4-(4-{[5-amino-7-(butylamino)-3- | | | |
| | methyl-1H-pyrazolo[4,3-d]pyrimidin- | | | |
| | 1-yl]methyl}-3-methoxybenzoyl)- | | | |
| | piperazin-2-one | | | |
| 120 | 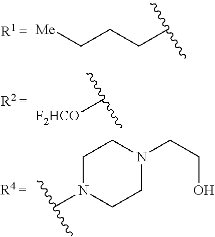 | 1,203.2 | 94.6 | [M + H]⁺ 519.0 RT (min)/Method 1.14/D δ 8.53-8.35 (m, 1H), 7.83 (s, 1H), 7.60-7.10 (m, 3H), 7.03 (d, J = 7.9 Hz, 1H), 5.88 (s, 2H), 3.76 (br t, J = 4.7 Hz, 2H), 3.21 (br s, 2H), 1.60 (quin, J = 7.2 Hz, 2H), 1.32-1.18 (m, 2H), 0.89 (t, J = 7.5 Hz, 3H) |
| | 2-[4-(4-{[5-amino-7-(butylamino)-1H- | | | |
| | pyrazolo[4,3-d]pyrimidin-1-yl]methyl}- | | | |
| | 3-(difluoromethoxy)benzoyl)- | | | |
| | piperazin-1-yl]ethan-1-ol | | | |
| 121 | 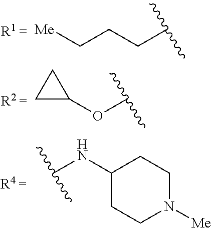 | 502.4 | 99.1 | [M + H]⁺ 493.2 RT (min)/Method 1.29/C 8.23 (br d, J = 7.3 Hz, 1H), 7.70 (s, 1H), 7.56 (s, 1H), 7.32 (br d, J = 7.6 Hz, 1H), 6.56 (br d, J = 7.9 Hz, 1H), 5.64 (s, 1H), 5.60 (s, 2H), 3.97 (br s, 2H), 3.75 (br s, 1H), 3.54 (br s, 1H), 3.39 (br d, J = 6.1 Hz, 2H), 2.83 (br d, J = 11.3 Hz, 2H), 2.22 (s, 3H), 2.08 (br t, J = 11.0 Hz, 2H), 1.77 (br d, J = 11.0 Hz, 2H), 1.67-1.54 (m, 2H), 1.54- 1.41 (m, 2H), 1.22-1.11 (m, 2H), 0.87-0.77 (m, 5H), 0.59 (br s, 2H) |
| | 4-{[5-amino-7-(butylamino)-1H- | | | |
| | pyrazolo[4,3-d]pyrimidin-1- | | | |
| | yl]methyl}-3-cyclopropoxy-N-(1- | | | |
| | methylpiperidin-4-yl)benzamide | | | |
| 122 | 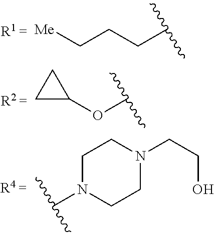 | 1,192.6 | 340.2 | [M + H]⁺ 509.1 RT (min)/Method 1.36/C δ 7.58 (s, 1H), 7.27 (s, 1H), 6.87 (br d, J = 7.3 Hz, 1H), 6.69 (br s, 1H), 6.55 (d, J = 7.6 Hz, 1H), 5.80 (br s, 1H), 5.60 (s, 2H), 3.95 (br s, 1H), 3.60-3.46 (m, 1H), 3.42 (br d, J = 6.1 Hz, 1H), 2.50-2.35 (m, 5H), 1.50 (quin, J = 7.2 Hz, 2H), 1.20 (sxt, J = 7.3 Hz, 2H), 0.84 (t, J = 7.3 Hz, 3H), 0.80 (br d, J = 6.4 Hz, 2H), 0.59 (br s, 2H) |
| | 2-[4-(4-{[5-amino-7-(butylamino)-1H- | | | |
| | pyrazolo[4,3-d]pyrimidin-1- | | | |
| | yl]methyl}-3-cyclopropoxybenzoyl) | | | |
| | piperazin-1-yl]ethan-1-ol | | | |
| 123 | 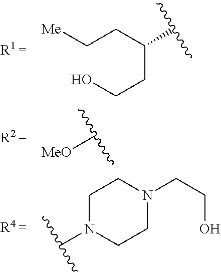 | 114.8 | 71.9 | [M + H]⁺ 527.3 RT (min)/Method 1.22/H δ 7.62 (s, 1H), 7.05 (s, 1H), 6.84 (d, J = 7.9 Hz, 1H), 6.40 (d, J = 7.9 Hz, 1H), 6.01-5.60 (m, 4H), 4.44-4.26 (m, 1H), 3.89 (s, 3H), 3.51 (br t, J = 5.6 Hz, 1H), 2.50-2.29 (m, 6H), 1.73- 1.62 (m, 1H), 1.61-1.50 (m, 1H), 1.43 (q, J = 6.8 Hz, 2H), 1.10 (tt, J = 14.4, 7.0 Hz, 2H), 0.78 (t, J = 7.3 Hz, 3H) |
| | (3S)-3-{[5-amino-1-({4-[4-(2-hydroxy- | | | |
| | ethyl)piperazine-1-carbonyl]-2- | | | |
| | methoxyphenyl}methyl)-1H-pyrazolo- | | | |
| | [4,3-d]pyrimidin-7-yl]amino}hexan- | | | |
| | 1-ol | | | |
| 124 | 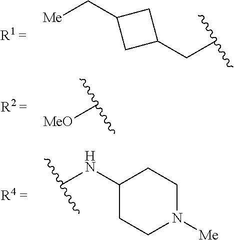 | 507.9 | 95.4 | [M + H]⁺ 507.3 RT (min)/Method 1.5/B 8.23 (br s, 1H), 7.59 (br s, 1H), 7.46 (br s, 1H), 7.31 (br s, 1H), 6.52- 6.38 (m, 1H), 5.75-5.59 (m, 4H), 3.91 (br s, 3H), 3.80-3.65 (m, 1H), 3.54 (br s, 1H), 3.37 (br s, 1H), 2.80 (br d, J = 10.1 Hz, 2H), 2.50-2.39 (m, 1H), 2.34 (br s, 1H), 2.19 (br s, 3H), 2.00 (br s, 2H), 1.74 (br s, 2H), 1.70- 1.43 (m, 4H), 1.34 (br s, 1H), 1.25- 1.09 (m, 2H), 0.76-0.64 (m, 3H) |
| | 4-[(5-amino-7-{[(3-ethylcyclo- | | | |
| | butyl)methyl]amino}-1H-pyrazolo- | | | |
| | [4,3-d]pyrimidin-1-yl)methyl]-3- | | | |
| | methoxy-N-(1-methylpiperidin-4- | | | |
| | yl)benzamide | | | |
| 125 | 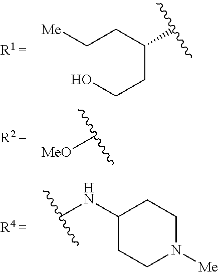 | 747.5 | 332.5 | [M + H]⁺ 511.1 RT (min)/Method 1.10/H δ 8.16 (br d, J = 7.6 Hz, 1H), 7.53 (s, 1H), 7.39 (s, 1H), 7.23 (br d, J = 7.9 Hz, 1H), 6.33 (br d, J = 7.9 Hz, 1H), 5.81-5.50 (m, 4H), 4.29-4.21 (m, 1H), 3.83 (s, 3H), 3.51-3.44 (m, 1H), 3.28 (br s, 1H), 2.77 (br d, J = 11.3 Hz, 2H), 2.16 (s, 3H), 2.02 (br t, J = 11.1 Hz, 2H), 1.69 (br d, J = 12.2 Hz, 2H), 1.62-1.41 (m, 6H), 1.32 (br dd, J = 9.0, 5.6 Hz, 1H), 1.24 (br dd, J = 14.5, 5.0 Hz, 1H), 0.96-0.81 (m, 2H), 0.64 (br t, J = 7.2 Hz, 3H) |
| | 4-[(5-amino-7-{[(3S)-1- | | | |
| | hydroxyhexan-3-yl]amino}-1H- | | | |
| | pyrazolo[4,3-d]pyrimidin-1- | | | |
| | yl)methyl]-3-methoxy-N-(1- | | | |
| | methylpiperidin-4-yl)benzamide | | | |
| 126 | 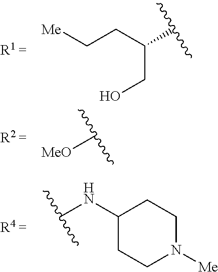 | 5,000.0 | 325.7 | [M + H]⁺ 497.1 RT (min)/Method 1.07/G δ 8.53-8.40 (m, 1H), 7.83-7.70 (m, 2H), 7.46 (s, 1H), 7.41-7.32 (m, 1H), 7.26-7.17 (m, 1H), 6.78 (br d, J = 8.0 Hz, 1H), 5.96-5.85 (m, 1H), 5.70 (d, J = 16.3 Hz, 1H), 4.48-4.34 (m, 1H), 4.08-3.94 (m, 1H), 3.89- 3.80 (m, 3H), 3.54-3.43 (m, 1H), 2.76 (s, 3H), 2.05-1.93 (m,2H), 1.84-1.69 (m, 2H), 1.59-1.47 (m, 1H), 1.45-1.33 (m, 1H), 1.11-0.98 (m, 2H), 0.77 (t, J = 7.3 Hz, 3H) |
| | 4-[(5-amino-7-{[(2S)-1- | | | |
| | hydroxypentan-2-yl]amino}-1H- | | | |
| | pyrazolo[4,3-d]pyrimidin-1- | | | |
| | yl)methyl]-3-methoxy-N-(1- | | | |
| | methylpiperidin-4-yl)benzamide | | | |
| 127 | 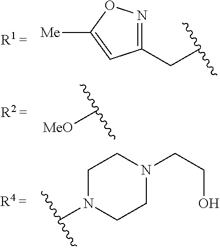 | 505 | 173.4 | LC/MS [M + H]⁺ = 522.2 RT (min) = 0.95 (LC/MS Procedure K) ¹H NMR (500 MHz, DMSO-d₆) δ 8.77-8.67 (m, 1H), 7.78-7.77 (m, 1H), 7.76-7.62 (m, 2H), 7.02 (s, 1H), 6.95-6.92 (m, 1H), 6.91-6.87 (m, 1H), 6.16 (s, 1H), 5.73 (s, 2H), 4.82-4.75 (m, 2H), 3.22-3.17 (m, 4H), 3.11 (br s, 2H), 2.35 (s, 3H) |
| | 2-(4-{4-[(5-amino-7-{[(5-methyl-1,2- | | | |
| | oxazo1-3-yl)methyl]amino}-1H- | | | |
| | pyrazolo[4,3-d]pyrimidin-1- | | | |
| | yl)methyl]-3-methoxybenzoyl}- | | | |
| | piperazin-1-yl)ethan-1-ol | | | |
| 128 | 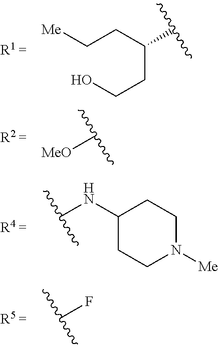 | 178.7 | 772 | LC/MS [M + H]⁺ 529.3 RT (min)/Method 1.21/B δ 8.23 (br d, J = 7.7 Hz, 1H), 7.43 (s, 1H), 7.32 (d, J = 7.8 Hz, 1H), 6.58 (d, J = 7.9 Hz, 1H), 6.18 (d, J = 8.5 Hz, 1H), 5.84 (br s, 2H), 5.60 (br d, J = 16.9 Hz, 1H), 5.44 (br d, J = 16.9 Hz, 1H), 4.34 (br d, J = 7.5 Hz, 1H), 3.85 (s, 3H), 3.81-3.65 (m, 2H), 3.37 (br t, J = 6.2 Hz, 1H), 2.82 (br d, J = 11.7 Hz, 2H), 2.21 (s, 3H), 2.07 (br t, J = 11.6 Hz, 2H), 1.75 (br d, J = 11.5 Hz, 2H), 1.71-1.53 (m, 4H), 1.41 (dt, J = 14.7, 7.3 Hz, 2H), 1.04 (br d, J = 6.3 Hz, 2H), 0.74 (t, J = 7.4 Hz, 3H). |
| | 4-[(5-amino-3-fluoro-7-{[(3S)-1- | | | |
| | hydroxyhexan-3-yl]amino}-1H- | | | |
| | pyrazolo[4,3-d]pyrimidin-1- | | | |
| | yl)methyl]-3-methoxy-N-(1- | | | |
| | methylpiperidin-4-yl)benzamide | | | |
| 129 | 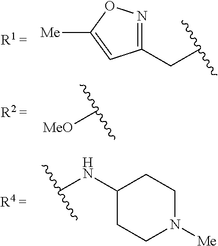 | 1235.1 | 67.7 | LC/MS [M + H]⁺ 506.1 RT (min/Method) = 0.94/Method K δ 8.48 (td, J = 5.7, 2.7 Hz, 1H), 7.81 (br s, 1H), 7.78 (s, 1H), 7.43-7.39 (m, 1H), 7.37 (br d, J = 7.6 Hz, 1H), 6.86 (br d, J = 7.5 Hz, 1H), 6.11 (s, 1H), 5.76 (s, 1H), 4.77 (br d, J = 4.0 Hz, 2H), 4.06-3.95 (m, 1H), 3.76 (s, 3H), 3.14-3.01 (m, 2H), 2.76 (s, 3H), 2.34 (s, 3H), 2.07-1.95 (m, 2H), 1.77 (br d, J = 7.8 Hz, 2H) |
| | 4-[(5-amino-7-{[(5-methyl-1,2- | | | |
| | oxazo1-3-yl)methyl]amino}-1H- | | | |
| | pyrazolo[4,3-d]pyrimidin-1- | | | |
| | yl)methyl]-3-methoxy-N-(1- | | | |
| | methylpiperidin-4-yl)benzamide | | | |
| 130 | 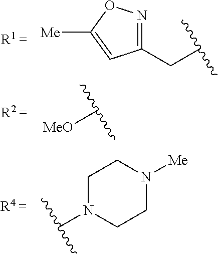 | 144.9 | 15.8 | LC/MS [M + H]⁺ 491.9 RT (min/Method) = 1.03/Method K δ 7.76 (s, 1H), 7.04-7.02 (m, 1H), 6.96-6.91 (m, 1H), 6.91-6.86 (m, 1H), 6.17 (s, 1H), 5.73 (s, 2H), 4.78 (br d, J = 3.5 Hz, 2H), 3.71 (br s, 3H), 3.23-3.15 (m, 4H), 2.77 (br s, 3H), 2.35 (s, 3H) |
| | 1-{[2-methoxy-4-(4-methylpiperazine- | | | |
| | 1-carbonyl)phenyl]methyl}-N7- | | | |
| | [(5-methyl-1,2-oxazo1-3-yl)methyl]- | | | |
| | 1H-pyrazolo[4,3-d]pyrimidine-5,7- | | | |
| | diamine | | | |
| 131 | 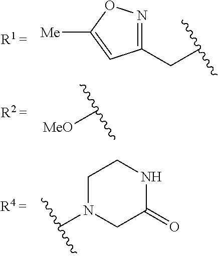 | 2187.9 | 363.1 | LC/MS [M + H]⁺ 492.3 RT (min/Method) = 1.00/Method K δ 8.11 (br s, 1H), 7.64 (s, 1H), 7.55- 7.44 (m, 1H), 7.09-7.03 (m, 1H), 6.89 (d, J = 7.6 Hz, 1H), 6.54 (br d, J = 7.7 Hz, 1H), 6.00 (br s, 2H), 5.68 (s, 2H), 4.65 (br d, J = 2.3 Hz, 2H), 4.26-3.94 (m, 2H), 3.79 (s, 3H), 3.31-3.11 (m, 4H), 2.33 (s, 3H) |
| | 4-{4-[(5-amino-7-{[(5-methyl-1,2- | | | |
| | oxazo1-3-yl)methyl]amino}-1H- | | | |
| | pyrazolo[4,3-d]pyrimidin-1- | | | |
| | yl)methyl]-3-methoxybenzoyl}- | | | |
| | piperazin-2-one | | | |
| 132 | 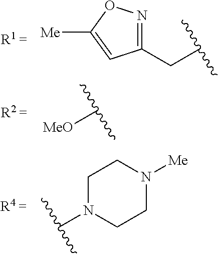 | 95.1 | 10.9 | LC/MS [M + H]⁺ 593.2 RT (min/Method) = 0.90/Method K δ 7.62 (s, 1H), 7.44 (br s, 1H), 6.99- 6.96 (m, 1H), 6.82 (d, J = 7.6 Hz, 1H), 6.64 (br d, J = 7.6 Hz, 1H), 5.80 (br s, 2H), 5.65 (s, 2H), 4.77 (br d, J = 4.6 Hz, 2H), 3.82 (s, 3H), 3.69-3.55 (m, 4H), 2.45-2.28 (m, 4H), 2.22 (br s, 3H) |
| | 1-{[2-methoxy-4-(4-methylpiperazine- | | | |
| | 1-carbonyl)phenyl]methyl}- | | | |
| | N7-[(5-methyl-1,2,4-oxadiazo1-3- | | | |
| | yl)methyl]-1H-pyrazolo[4,3- | | | |
| | d]pyrimidine-5,7-diamine | | | |
| 133 | 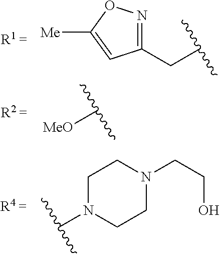 | 1869.7 | 552 | LC/MS [M + H]⁺ 523.3 RT (min/Method) = 0.97/Method C δ 7.62 (s, 1H), 7.41 (br d, J = 5.1 Hz, 1H), 6.99-6.97 (m, 1H), 6.82 (dd, J = 7.7, 1.1 Hz, 1H), 6.62 (d, J = 7.7 Hz, 1H), 5.77 (br s, 2H), 5.66 (s, 2H), 4.77 (br d, J = 5.2 Hz, 2H), 4.57-4.42 (m, 1H), 3.82 (s, 3H), 3.64-3.55 (m, 2H), 3.50 (br t, J = 6.0 Hz, 2H), 3.32- 3.29 (m, J = 8.7 Hz, 2H), 2.54 (s, 3H), 2.48-2.45 (m, 2H), 2.43 (br t, J = 5.9 Hz, 2H), 2.38 (br dd, J = 8.9, 6.9 Hz, 2H) |
| | 2-(4-{4-[(5-amino-7-{[(5-methyl- | | | |
| | 1,2,4-oxadiazo1-3-yl)methyl]amino}- | | | |
| | 1H-pyrazolo[4,3-d]pyrimidin-1- | | | |
| | yl)methyl]-3-methoxybenzoyl}- | | | |
| | piperazin-1-yl)ethan-1-ol | | | |

### Pharmaceutical Compositions and Administration

In another aspect, there is provided a pharmaceutical composition comprising a compound of as disclosed herein, or of a conjugate thereof, formulated together with a pharmaceutically acceptable carrier or excipient. It may optionally contain one or more additional pharmaceutically active ingredients, such as a biologic or a small molecule drug. The pharmaceutical compositions can be administered in a combination therapy with another therapeutic agent, especially an anti-cancer agent.

The pharmaceutical composition may comprise one or more excipients. Excipients that may be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003).

Preferably, a pharmaceutical composition is suitable for intravenous, intramuscular, subcutaneous, parenteral, spinal or epidermal administration (e.g., by injection or infusion). Depending on the route of administration, the active compound may be coated in a material to protect it from the action of acids and other natural conditions that may inactivate it. The phrase “parenteral administration” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the pharmaceutical composition can be administered via a non-parenteral route, such as a topical, epidermal or mucosal route of administration, for example, intranasally, orally, vaginally, rectally, sublingually or topically.

Pharmaceutical compositions can be in the form of sterile aqueous solutions or dispersions. They can also be formulated in a microemulsion, liposome, or other ordered structure suitable to achieve high drug concentration. The compositions can also be provided in the form of lyophilates, for reconstitution in water prior to administration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide a therapeutic response. For example, a single bolus may be administered, several divided doses may be administered over time, or the dose may be proportionally reduced or increased as indicated by the exigencies of the situation. It is especially advantageous to formulate parenteral compositions in dosage unit form for ease of administration and uniformity of dosage. “Dosage unit form” refers to physically discrete units suited as unitary dosages for the subjects to be treated; each unit containing a predetermined quantity of active compound calculated to produce the desired therapeutic response, in association with the required pharmaceutical carrier.

The dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg, or alternatively 0.1 to 5 mg/kg. Exemplary treatment regimens are administration once per week, once every two weeks, once every three weeks, once every four weeks, once a month, once every 3 months, or once every three to 6 months. Preferred dosage regimens include 1 mg/kg body weight or 3 mg/kg body weight via intravenous administration, using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/mL and in some methods about 25-300 μg/mL.

A “therapeutically effective amount” of a compound of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective amount” preferably inhibits tumor growth by at least about 20%, more preferably by at least about 40%, even more preferably by at least about 60%, and still more preferably by at least about 80% relative to untreated subjects. A therapeutically effective amount of a therapeutic compound can decrease tumor size, or otherwise ameliorate symptoms in a subject, which is typically a human but can be another mammal. Where two or more therapeutic agents are administered in a combination treatment, “therapeutically effective amount” refers to the efficacy of the combination as a whole, and not each agent individually.

The pharmaceutical composition can be a controlled or sustained release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices; (2) micro-infusion pumps; (3) transdermal devices; (4) infusion devices; and (5) osmotic devices.

In certain embodiments, the pharmaceutical composition can be formulated to ensure proper distribution in vivo. For example, to ensure that the therapeutic compounds of the invention cross the blood-brain barrier, they can be formulated in liposomes, which may additionally comprise targeting moieties to enhance selective transport to specific cells or organs.

### INDUSTRIAL APPLICABILITY AND USES

TLR7 agonist compounds disclosed herein can be used for the treatment of a disease or condition that can be ameliorated by activation of TLR7.

In one embodiment, the TLR7 agonist is used in combination with an anti-cancer immunotherapy agent—also known as an immuno-oncology agent. An anti-cancer immunotherapy agent works by stimulating a body's immune system to attack and destroy cancer cells, especially through the activation of T cells. The immune system has numerous checkpoint (regulatory) molecules, to help maintain a balance between its attacking legitimate target cells and preventing it from attacking healthy, normal cells. Some are stimulators (up-regulators), meaning that their engagement promotes T cell activation and enhances the immune response. Others are inhibitors (down-regulators or brakes), meaning that their engagement inhibits T cell activation and abates the immune response. Binding of an agonistic immunotherapy agent to a stimulatory checkpoint molecule can lead to the latter's activation and an enhanced immune response against cancer cells. Reciprocally, binding of an antagonistic immunotherapy agent to an inhibitory checkpoint molecule can prevent down-regulation of the immune system by the latter and help maintain a vigorous response against cancer cells. Examples of stimulatory checkpoint molecules are B7-1, B7-2, CD28, 4-1BB (CD137), 4-1BBL, ICOS, CD40, ICOS-L, OX40, OX40L, GITR, GITRL, CD70, CD27, CD40, DR3 and CD28H. Examples of inhibitory checkpoint molecules are CTLA-4, PD-1, PD-L1, PD-L2, LAG-3, TIM-3, Galectin 9, CEACAM-1, BTLA, CD69, Galectin-1, CD113, GPR56, VISTA, 2B4, CD48, GARP, PD1H, LAIR1, TIM-1, CD96 and TIM-4.

Whichever the mode of action of an anti-cancer immunotherapy agent, its effectiveness can be increased by a general up-regulation of the immune system, such as by the activation of TLR7. Thus, in one embodiment, this specification provides a method of treating a cancer, comprising administering to a patient suffering from such cancer a therapeutically effective combination of an anti-cancer immunotherapy agent and a TLR7 agonist as disclosed herein. The timing of administration can be simultaneous, sequential, or alternating. The mode of administration can systemic or local. The TLR7 agonist can be delivered in a targeted manner, via a conjugate.

Cancers that could be treated by a combination treatment as described above include acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, testicular cancer, throat cancer, thyroid cancer, urethral cancer, uterine cancer, vaginal cancer, and vulvar cancer.

Anti-cancer immunotherapy agents that can be used in combination therapies as disclosed herein include: AMG 557, AMP-224, atezolizumab, avelumab, BMS 936559, cemiplimab, CP-870893, dacetuzumab, durvalumab, enoblituzumab, galiximab, IMP321, ipilimumab, lucatumumab, MEDI-570, MEDI-6383, MEDI-6469, muromonab-CD3, nivolumab, pembrolizumab, pidilizumab, spartalizumab, tremelimumab, urelumab, utomilumab, varlilumab, vonlerolizumab. Table B below lists their alternative name(s) (brand name, former name, research code, or synonym) and the respective target checkpoint molecule.

TABLE B

| Immunotherapy Agent | Alternative Name(s) | Target |
|---|---|---|
| AMG 557 | | B7RP-1 (ICOSL) |
| AMP-224 | | PD-1 |
| Atezolizumab | MPDL3280A, RO5541267, | PD-L1 |
| | TECENTRIQ ® | |
| Avelumab | BAVENCIO ® | PD-L1 |
| BMS 936559 | | PD-L1 |
| Cemiplimab | LIBTAYO ® | PD-1 |
| CP-870893 | | CD40 |
| Dacetuzumab | | CD40 |
| Durvalumab | IMFINZI ® | PD-L1 |
| Enoblituzumab | MGA271 | B7-H3 |
| Galiximab | | B7-1 (CD80) |
| IMP321 | | LAG-3 |
| Ipilimumab | YERVOY ® | CTLA-4 |
| Lucatumumab | | CD40 |
| MEDI-570 | | ICOS (CD278) |
| MEDI-6383 | | OX40 |
| MEDI-6469 | | OX40 |
| Muromonab-CD3 | | CD3 |
| Nivolumab | OPDIVO ® | PD-1 |
| Pembrolizumab | KEYTRUDA ® | PD-1 |
| Pidilizumab | MDV9300 | PD-1 |
| Spartalizumab | PDR001 | PD-1 |
| Tremelimumab | Ticilimumab, CP-675, CP- | CTLA-4 |
| | 675,206 | |
| Urelumab | BMS-663513 | CD137 |
| Utomilumab | PF-05082566 | CD137 |
| Varlilumab | CDX 1127 | CD27 |
| Vonlerolizumab | RG7888, MOXR0916, | OX40 |
| | pogalizumab | |

In one embodiment of a combination treatment with a TLR7 agonist, the anti-cancer immunotherapy agent is an antagonistic anti-CTLA-4, anti-PD-1, or anti-PD-L1 antibody. The cancer can be lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

In another embodiment of a combination treatment with a TLR7 agonist, the anti-cancer immunotherapy agent is an antagonistic anti-CTLA-4 antibody, preferably ipilimumab.

In another embodiment of a combination treatment with a TLR7 agonist, the anti-cancer immunotherapy agent is an antagonistic anti-PD-1 antibody, preferably nivolumab or pembrolizumab.

The TLR7 agonists disclosed herein also are useful as vaccine adjuvants.

The practice of this invention can be further understood by reference to the following examples, which are provided by way of illustration and not of limitation.

### Analytical Procedures

### NMR

The following conditions were used for obtaining proton nuclear magnetic resonance (NMR) spectra: NMR spectra were taken in either 400 Mz or 500 Mhz Bruker instrument using either DMSO-d6 or CDCl₃ as solvent and internal standard. The crude NMR data was analyzed by using either ACD Spectrus version 2015-01 by ADC Labs or MestReNova software.

Chemical shifts are reported in parts per million (ppm) downfield from internal tetramethylsilane (TMS) or from the position of TMS inferred by the deuterated NMR solvent. Apparent multiplicities are reported as: singlet-s, doublet-d, triplet-t, quartet-q, or multiplet-m. Peaks that exhibit broadening are further denoted as br. Integrations are approximate. It should be noted that integration intensities, peak shapes, chemical shifts and coupling constants can be dependent on solvent, concentration, temperature, pH, and other factors. Further, peaks that overlap with or exchange with water or solvent peaks in the NMR spectrum may not provide reliable integration intensities. In some cases, NMR spectra may be obtained using water peak suppression, which may result in overlapping peaks not being visible or having altered shape and/or integration.

### Liquid Chromatography

The following liquid chromatography methods were used:

LC/MS Method A. Column: XBridge C18, 200 mm×19 mm, 5-μm particles; Mobile Phase A: 5:95 acetonitrile: water with ammonium acetate; Mobile Phase B: 95:5 acetonitrile:water with ammonium acetate; Gradient: a 0-minute hold at 6% B, 6-46% B over 20 min, then a 0-minute hold at 100% B; Flow Rate: 20 mL/min; Column temperature: 25° C. Fraction collection was triggered by MS signals.

LC/MS Method B. Column: XBridge C18, 200 mm×19 mm, 5-μm particles; Mobile Phase A: 5:95 acetonitrile: water with ammonium acetate; Mobile Phase B: 95:5 acetonitrile:water with ammonium acetate; Gradient: a 0-minute hold at 11% B, 11-51% B over 25 min, then a 0-minute hold at 100% B; Flow Rate: 20 mL/min; Column Temperature: 25° C. Fraction collection was triggered by MS signals.

LC/MS Method C. Column: XBridge C18, 200 mm×19 mm, 5-μm particles; Mobile Phase A: 5:95 acetonitrile: water with ammonium acetate; Mobile Phase B: 95:5 acetonitrile:water with ammonium acetate; Gradient: a 0-minute hold at 2% B, 2-42% B over 20 min, then a 0-minute hold at 100% B; Flow Rate: 20 mL/min; Column Temperature: 25° C. Fraction collection was triggered by MS signals.

LC/MS Method D. Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 5% B, 5-45% B over 20 min, then a 0-minute hold at 100% B; Flow Rate: 20 mL/min; Column Temperature: 25° C. Fraction collection was triggered by MS and UV signals.

LC/MS Method E. Column: XBridge C18, 200 mm×19 mm, 5-μm particles; Mobile Phase A: 5:95 acetonitrile: water with ammonium acetate; Mobile Phase B: 95:5 acetonitrile:water with ammonium acetate; Gradient: a 0-minute hold at 3% B, 3-43% B over 20 min, then a 0-minute hold at 100% B; Flow Rate: 20 mL/min; Column Temperature: 25° C. Fraction collection was triggered by MS and UV signals.

LC/MS Method F. Column: Acquity BEH C18, 2.1 mm×50 mm, 1.7 μm particles; Mobile Phase A: water with with 0.05% TFA; Mobile Phase B: acetonitrile with 0.05% TFA; Gradient: 2% B to 98% B over 1 min, then a 0.5 min hold at 98% B; Flow: 0.8 mL/min; Detection: MS and UV.

LC/MS Method G. Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min.

LC/MS Method H. Column: XBridge C18, 200 mm×19 mm, 5-μm particles; Mobile Phase A: 5:95 acetonitrile:water with ammonium acetate; Mobile Phase B: 95:5 acetonitrile:water with ammonium acetate; Gradient: a 0-minute hold at 5% B, 5-45% B over 20 min, then a 0-minute hold at 100% B; Flow Rate: 20 mL/min; Column Temperature: 25° C. Fraction collection was triggered by MS signals.

LC/MS Method I. Column: XBridge C18, 200 mm×19 mm, 5-μm particles; Mobile Phase A: 5:95 acetonitrile: water with ammonium acetate; Mobile Phase B: 95:5 acetonitrile:water with ammonium acetate; Gradient: a 0-minute hold at 0% B, 0-40% B over 20 min, then a 4-minute hold at 100% B; Flow Rate: 20 mL/min; Column Temperature: 25° C. Fraction collection was triggered by MS signals.

LC/MS Method J. Column: BEH C18 2.1×50 mm; Mobile Phase A: water with 0.05% TFA; Mobile Phase B: acetonitrile with 0.05% TFA; Temperature: 50° C.; Gradient: 2-98% B over 1.0 min, then a 0.50 min hold at 98% B; Flow: 0.8 mL/min. Detection: MS and UV (220 nm).

LC/MS Method K. Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm).

LC/MS Method L. Column: BEH C18 2.1×50 mm; Mobile Phase A: 95:5 H₂O:ACN with 0.01M NH4OAc; Mobile Phase B: 5:95 H₂O:ACN with 0.01M NH4OAc; Temperature: 50° C.; Gradient: 5-95% B over 1 min; Flow: 0.8 mL/min.

### Synthesis—General Procedures

Generally, the procedures disclosed herein produce a mixture of regioisomers, alkylated at the 1H or 2H position of the pyrazolopyrimidine ring system (which are also referred to as N1 and N2 regioisomers, respectively, alluding to the nitrogen that is alkylated). For brevity, the N2 regioisomers are not shown, but it is to be understood that they are present in the initial product mixture and separated at a later time, for example by preparative HPLC.

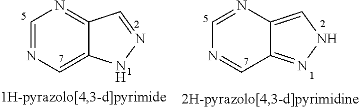 [SMILES: c1ncc2[nH]ncc2n1 ; c1ncc2n[nH]cc2n1]

The mixture of regioisomers can be separated at an early stage of the synthesis and the remaining synthetic steps carried out with the 1H regioisomer or, alternatively, the synthesis can be progressed carrying the mixture of regioisomers and separation effected at a later stage, as desired.

The compounds of the present disclosure can be prepared by a number of methods well known to one skilled in the art of synthetic organic chemistry. These methods include those described below, or variations thereof. Preferred methods include, but are not limited to, those described below in the Schemes below. The Schemes are intended to be generic, but in some instances a feature may be depicted specifically (e.g., a methyl ester or particular regioisomer) as a matter of convenience.

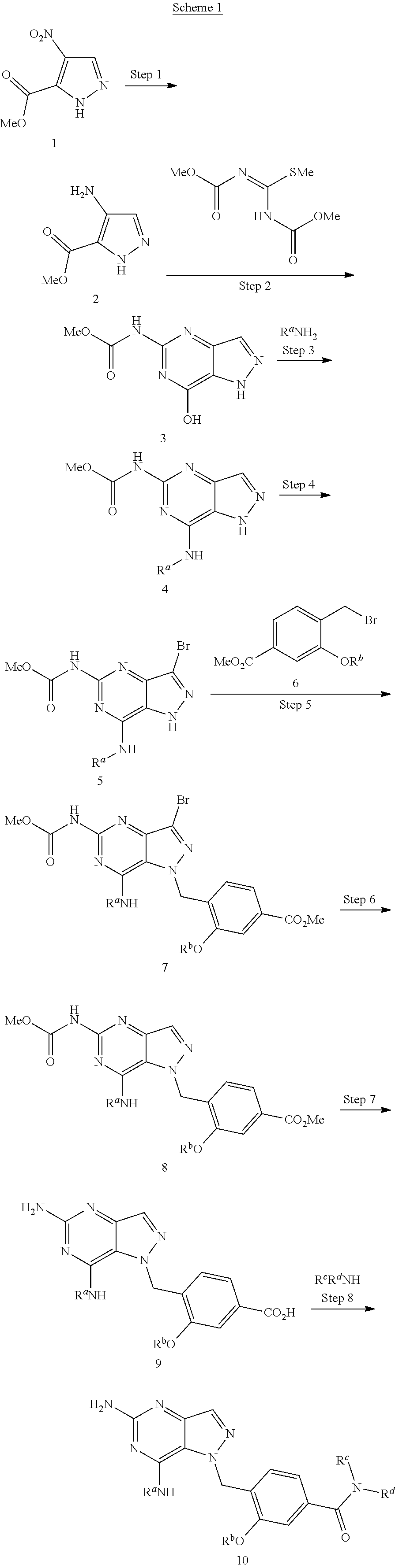

R^{a} can be, in Scheme 1 and other occurrences thereof, for example,

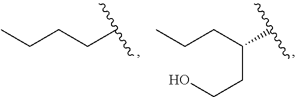

or other suitable moiety. R^{b} is, in Scheme 1 and other occurrences thereof, for example, C₁-C₃ alkyl. R^{c}NHR^{d} is, in Scheme 1 and other occurrences thereof, a primary or secondary amine. R^{a}, R^{b}, R^{c}, and/or R^{d} can have functional groups masked by a protecting group that is removed at the appropriate time during the synthetic process.

Compound 10 can be prepared by the synthetic sequence outlined in Scheme 1 above. Reduction of nitropyrazole 1 to afford compound 2 followed by cyclization with 1,3-bis(methoxycarbonyl)-2-methyl-2-thiopseudourea gives the hydroxypyrazolopyrimidine 3. The amine R^{a}NH₂ is introduced using BOP/DBU coupling conditions, and the subsequent bromination using NBS (Step 4) gives the bromopyrazolopyrimidine 5. Alkylation using a benzyl halide 6 gives a mixture of N1 and N2 products, which are separated, giving N1 intermediate 7. Catalytic hydrogenation (step 6) followed by a one-pot methyl carbamate deprotection and saponification gives the intermediate acid 9. Coupling of the acid 9 with an amine using HATU (or EDC) conditions gives the target compound 10. (Alkylation of brominated intermediate 5 in Step 5 gives a better ratio of N1/N2 product, compared to alkylation of unbrominated intermediate 4).

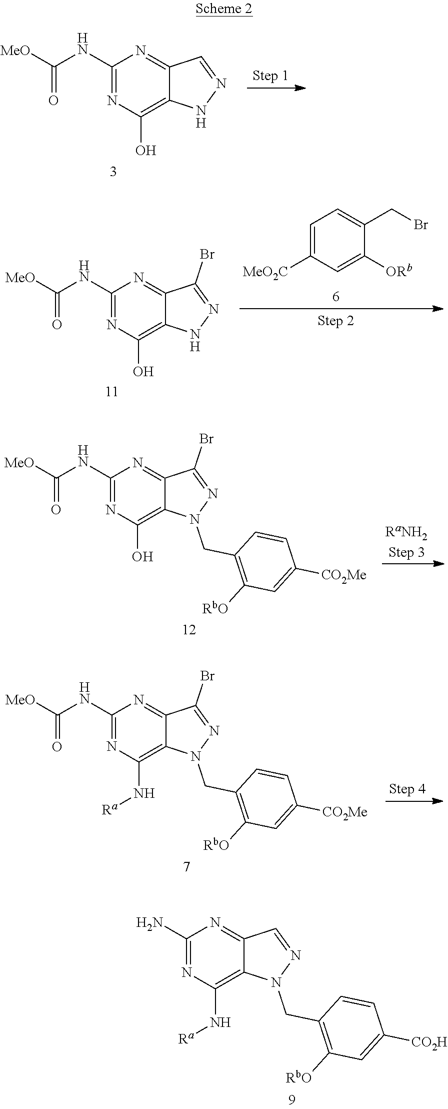

Alternatively, intermediate 9 may be accessed using the route described in Scheme 2 above. Intermediate 3 is brominated using NBS, then alkylated to give the intermediate ester 12. Amination then follows, using BOP coupling conditions to give intermediate 7. Catalytic hydrogenation followed by saponification and methyl carbamate deprotection gives intermediate 9.

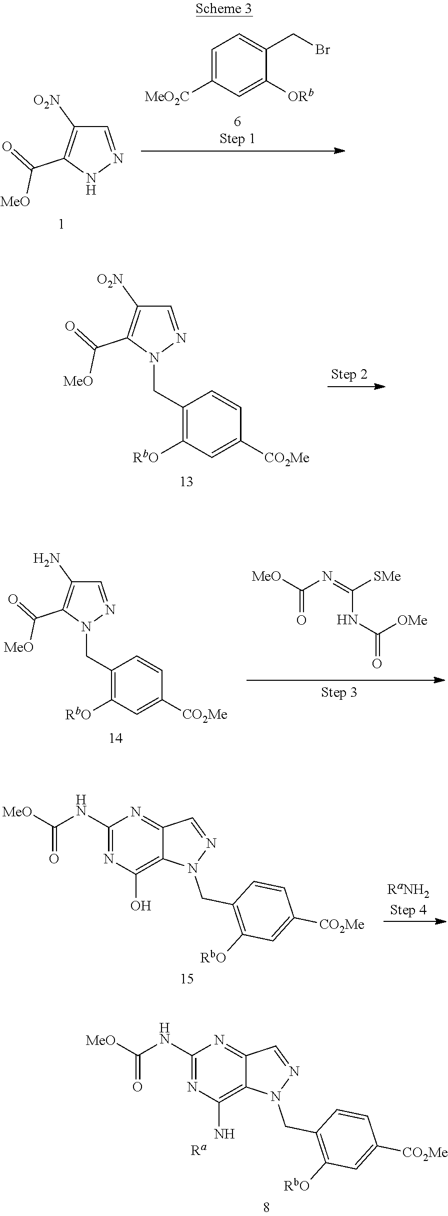

An alternative route to intermediate 8 begins with the alkylation of nitropyrazole 1 with benzyl halide 6, giving the benzyl pyrazole 13. Reduction of the nitro group followed by cyclisation with 1,3-bis(methoxycarbonyl)-2-methyl-2-thiopseudourea gives the hydroxypyrazolopyrimidine 15, which is converted to the appropriate amine derivative 8 using BOP/DBU conditions. This is illustrated in scheme 3 above.

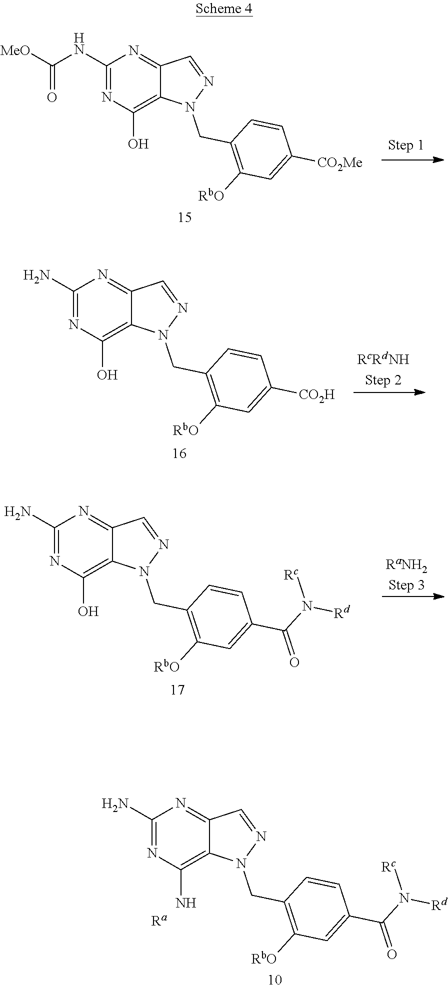

Another alternative route to the target compounds is shown in scheme 4 above. From intermediate 15, the ester group is hydrolysed and the methyl carbamate removed using sodium hydroxide, giving the acid 16. Amide coupling using HATU (or EDC) gives the amide 17, and subsequent amination using BOP/DBU conditions gives the target molecule 10.

### SYNTHESIS-SPECIFIC EXAMPLES

To further illustrate the foregoing, the following non-limiting, the following exemplary synthetic schemes are included. Variations of these examples within the scope of the the claims are within the purview of one skilled in the art and are considered to fall within the scope of this disclosure. The reader will recognize that the skilled artisan, provided with the present disclosure and skilled in the relevant art, will be able to prepare and use the compounds disclosed herein without exhaustive examples.

Analytical data for compounds numbered 100 and higher is found in Table A.

### Example 1—Compound 111

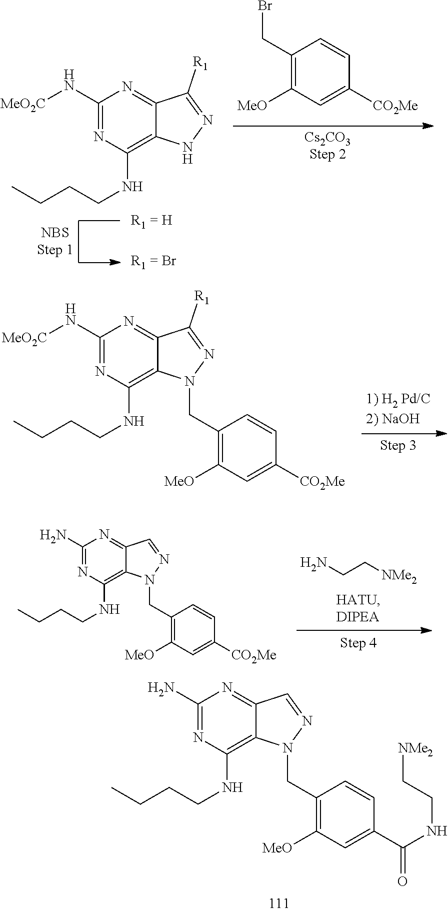

Step 1. A solution of NBS (6.94 g, 39.0 mmol) in DMF (20 mL) was added to a stirred suspension of methyl (7-(butylamino)-1H-pyrazolo[4,3-d]pyrimidin-5-yl)carbamate (10 g, 37.8 mmol) in DMF (80 mL). The reaction mixture was stirred at RT for 90 min and poured into water (400 mL) and stirred for 5 min. The product was collected by filtration, washed with water (200 mL), and left to air dry overnight, giving methyl (3-bromo-7-(butylamino)-1H-pyrazolo[4,3-d]pyrimidin-5-yl)carbamate (7.5 g, 21.85 mmol, 57.8% yield) as a solid.

LC-MS (ES, m/z): [M+H]⁺=343.0, 345.0.

¹H NMR (400 MHz, DMSO-d₆) δ 12.87 (br s, 1H), 9.80 (s, 1H), 7.56 (br s, 1H), 3.62 (s, 3H), 3.54 (q, J=6.6 Hz, 2H), 1.62 (quin, J=7.2 Hz, 2H), 1.40 (dq, J=14.8, 7.4 Hz, 2H), 0.94 (t, J=7.4 Hz, 3H).

Step 2. A solution of methyl 4-(bromomethyl)-3-methoxybenzoate (1.861 g, 7.18 mmol) in DMF (5 mL) was added portionwise over 5 min to a stirred suspension of methyl (3-bromo-7-(butylamino)-1H-pyrazolo[4,3-d]pyrimidin-5-yl)carbamate (2.9 g, 8.45 mmol) and Cs₂CO₃ (3.30 g, 10.14 mmol) in DMF (35 mL) at 0° C. The reaction mixture was warmed to RT, stirred overnight, poured into saturated NaHCO₃ solution (300 mL), and extracted with EtOAc (3×70 mL). The combined organic phases were washed with brine (4×50 mL), dried (MgSO₄), filtered, and concentrated. Flash chromatography (SiO₂ column, 0 to 50% EtOAc in hexanes) gave methyl 4-((3-bromo-7-(butylamino)-5-((methoxycarbonyl)amino)-1H-pyrazolo[4,3-d]pyrimidin-1-yl)methyl)-3-methoxybenzoate (1.400 g, 2.69 mmol, 31.8% yield) as a solid.

LC-MS (ES, m/z): [M+H]⁺=521.2, 523.2.

¹H NMR (400 MHz, DMSO-d₆) δ 9.88 (s, 1H), 7.54-7.48 (m, 2H), 7.32 (t, J=5.6 Hz, 1H), 6.79 (d, J=7.7 Hz, 1H), 5.78 (s, 2H), 3.86 (s, 3H), 3.85 (s, 3H), 3.63 (s, 3H), 3.52 (q, J=6.6 Hz, 2H), 1.56 (quin, J=7.3 Hz, 2H), 1.28-1.15 (m, 2H), 0.84 (t, J=7.4 Hz, 3H).

Step 3. Methyl 4-((3-bromo-7-(butylamino)-5-((methoxycarbonyl)amino)-1H-pyrazolo[4,3-d]pyrimidin-1-yl)methyl)-3-methoxybenzoate (1.400 g, 2.69 mmol) was suspended in EtOH (80 mL). 10% Pd/C (200 mg) was added. The reaction vessel was evacuated and purged with H₂ six times. The reaction mixture was stirred for 1 h under a H₂ atmosphere. The reaction vessel was evacuated, purged with N₂, and filtered through CELITE™ medium, washing with EtOH (100 mL). The filtrate was evaporated to dryness and the residue was dissolved in dioxane (10 mL). NaOH (3.22 mL, 16.11 mmol) was added, and the reaction mixture stirred at 80° C. for 2 h and cooled to RT. The reaction mixture was diluted with water (10 mL) and acidified with 5N HCl and the dioxane was removed by evaporation. The residue was diluted with more water (20 mL), and the product was collected by filtration and washed with water and then acetonitrile, to give 4-((5-amino-7-(butylamino)-1H-pyrazolo[4,3-d]pyrimidin-1-yl)methyl)-3-methoxybenzoic acid (900 mg, 2.430 mmol, 90% yield) as a white solid.

LC-MS (ES, m/z): [M+H]⁺ 371.2.

Step 4. A 20 mL scintillation vial was charged with 4-((5-amino-7-(butylamino)-1H-pyrazolo[4,3-d]pyrimidin-1-yl)methyl)-3-methoxybenzoic acid (30 mg, 0.081 mmol), HATU (37.0 mg, 0.097 mmol), DMF (2 mL) and N,N-dimethylethane-1,2-diamine (7.14 mg, 0.081 mmol). DIPEA (0.035 mL, 0.202 mmol) was added. The reaction mixture was stirred at 65° C. overnight. The crude product was purified via preparative LC/MS under the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 6% B, 6-46% B over 20 min, then a 0-minute hold at 100% B; Flow Rate: 20 mL/min; Column temperature: 25° C. Fraction collection was triggered by MS signals. Fractions containing the desired product were combined and dried via centrifugal evaporation, giving Compound 111 (3.3 mg, 0.0075 mmol, 9.3%).

The following compounds were analogously prepared: Compound 102, Compound 103, Compound 104, Compound 105, Compound 106, Compound 107, Compound 108, Compound 109, Compound 110, Compound 112, Compound 113, Compound 114, Compound 115, and Compound 116.

### Example 2—Compound 124

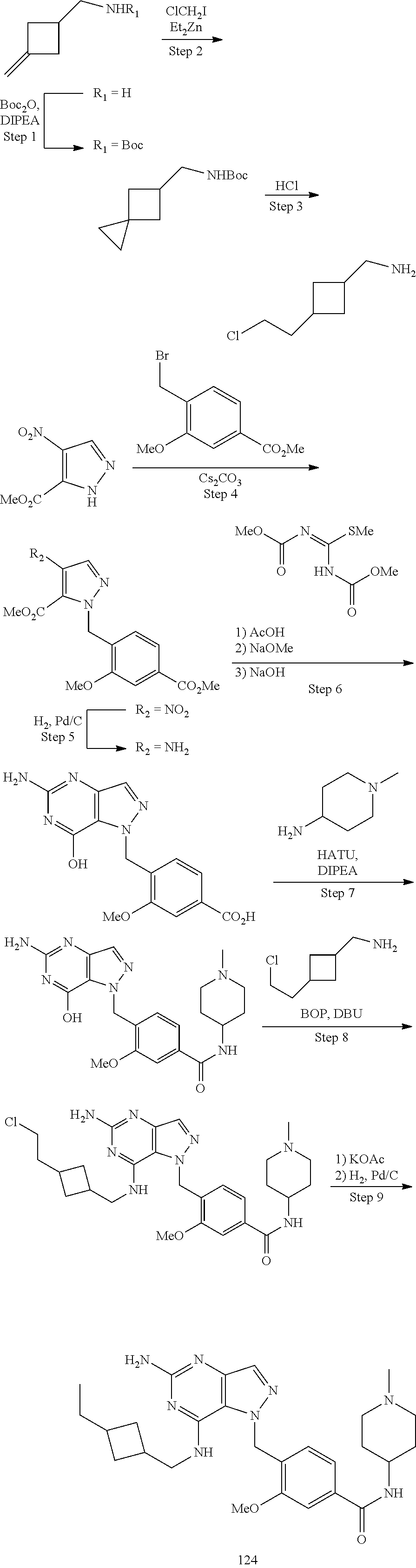

Step 1. (3-Methylenecyclobutyl)methanamine hydrochloride (4.5 g, 33.7 mmol) was suspended in DCM (30 mL). DIPEA (17.65 mL, 101 mmol) was added, followed by Boc-anhydride (8.60 mL, 37.0 mmol). The reaction mixture was stirred for 2 h at RT, poured into saturated NaHCO₃ solution (100 mL), and extracted with DCM (3×70 mL). The combined organic phases were washed with saturated NaHCO₃ solution (50 mL) and brine (4×50 mL), dried (MgSO₄), filtered and concentrated. Flash chromatography (SiO₂ column, 0 to 10% EtOAc in hexanes) gave tert-butyl ((3-methylenecyclobutyl)methyl)carbamate (4.73 g, 23.98 mmol, 71.2% yield) as an oil.

¹H NMR (400 MHz, CDCl₃) δ 4.77 (quin, J=2.3 Hz, 2H), 3.20 (br d, J=6.6 Hz, 2H), 2.81-2.72 (m, 2H), 2.48-2.32 (m, 3H), 1.45 (s, 9H).

Step 2. A solution of tert-butyl ((3-methylenecyclobutyl)methyl)carbamate (2.1 g, 10.64 mmol) in DCE (20 mL) was cooled in an ice bath. Chloroiodomethane (2.318 mL, 31.9 mmol) was added, followed by diethylzinc (15.97 mL, 15.97 mmol) portion-wise over 10 min. After the addition was complete, the reaction mixture was allowed to warm slowly to RT and stirred for 3 h. Water (5 mL) was added carefully to quench the reaction. The reaction mixture was acidified with 1N HCl (10 mL) and extracted with EtOAc (3×40 mL). The combined organic phases were washed with brine (10 mL), dried (MgSO₄), filtered and concentrated. Flash chromatography (SiO₂ column, 0 to 18% EtOAc in hexanes) gave tert-butyl (spiro[2.3]hexan-5-ylmethyl)carbamate (812 mg, 3.84 mmol, 36.1% yield) as an oil.

¹H NMR (400 MHz, CDCl₃-d) δ 4.53 (br s, 1H), 3.25 (br d, J=5.9 Hz, 2H), 2.53 (dt, J=14.6, 7.1 Hz, 1H), 2.18-2.11 (m, 2H), 1.81 (dd, J=12.4, 6.1 Hz, 2H), 1.45 (s, 9H), 0.39 (s, 4H).

Step 3. tert-Butyl (spiro[2.3]hexan-5-ylmethyl)carbamate (800 mg, 3.79 mmol) was dissolved in dioxane (3 mL) and HCl in dioxane (2.84 mL, 11.36 mmol) was added. The reaction mixture was stirred overnight at RT and evaporated to dryness, giving (3-(2-chloroethyl)-cyclobutyl)methanamine hydrochloride (680 mg, 3.7 mmol, 97% yield) as a solid.

¹H NMR (400 MHz, DMSO-d₆) δ 7.97 (br s, 4H), 3.54-3.39 (m, 1H), 3.03-2.91 (m, 1H), 2.91-2.79 (m, 2H), 2.54-2.45 (m, 3H), 2.45-2.26 (m, 3H), 2.23-2.15 (m, 1H), 1.91-1.79 (m, 3H), 0.98 (t, J=7.2 Hz, 2H), 0.92 (t, J=7.3 Hz, 2H).

Step 4. Cs₂CO₃ (11.42 g, 35.1 mmol) was added to a stirred solution of methyl 4-nitro-1H-pyrazole-5-carboxylate (5 g, 29.2 mmol) in DMF (30 mL). After cooling in an ice bath, a solution of methyl 4-(bromomethyl)-3-methoxybenzoate (7.57 g, 29.2 mmol) in DMF (20 mL) was added portion-wise over 5 min. The reaction mixture was allowed to warm slowly to RT, stirred overnight, poured into water (150 mL), and extracted with EtOAc (3×70 mL). The combined organic phases were washed with brine (4×50 mL), dried (MgSO₄), filtered and concentrated. Flash chromatography (SiO₂ column, 0 to 50% EtOAc in hexanes) gave methyl 1-(2-methoxy-4-(methoxycarbonyl)benzyl)-4-nitro-1H-pyrazole-5-carboxylate (1.012 g, 2.90 mmol, 9.92% yield), as a solid.

LC-MS (ES, m/z): [M+H]⁺ 350.1.

¹H NMR (400 MHz, DMSO-d₆) δ 8.40 (s, 1H), 7.57 (d, J=7.6 Hz, 1H), 7.50 (s, 1H), 7.27 (d, J=7.9 Hz, 1H), 5.53 (s, 2H), 3.96 (s, 3H), 3.86 (s, 3H), 3.82 (s, 3H).

Step 5. Methyl 1-(2-methoxy-4-(methoxycarbonyl)benzyl)-4-nitro-1H-pyrazole-5-carboxylate (2 g, 5.73 mmol) was suspended in EtOH (100 mL). 10% Pd/C (100 mg) was added. The reaction vessel was evacuated and purged six times with hydrogen. The reaction mixture was stirred overnight under a hydrogen atmosphere, filtered through CELITE™ medium, washing with EtOH (100 mL). The filtrate was evaporated to dryness, giving methyl 4-amino-1-(2-methoxy-4-(methoxycarbonyl)benzyl)-1H-pyrazole-5-carboxylate (1.764 g, 5.52 mmol, 96% yield) as a solid.

LC-MS (ES, m/z): [M+H]⁺ 320.1.

1H NMR (400 MHz, DMSO-d₆) δ 7.50 (s, 1H), 7.46 (d, J=7.7 Hz, 1H), 7.18 (s, 1H), 6.42 (d, J=7.9 Hz, 1H), 5.55 (s, 2H), 5.14 (s, 2H), 3.91 (s, 3H), 3.84 (s, 3H), 3.70 (s, 3H).

Step 6. Methyl 4-amino-1-(2-methoxy-4-(methoxycarbonyl)benzyl)-1H-pyrazole-5-carboxylate (1.75 g, 5.48 mmol) was suspended in MeOH (60 mL). 1,3-bis(methoxycarbonyl)-2-methyl-2-thiopseudourea (1.243 g, 6.03 mmol) was added, followed by AcOH (1.882 mL, 32.9 mmol). The reaction mixture was stirred for 1 h at RT. TFA (2 mL, 26 mmol) was added, and the reaction mixture was stirred overnight. Sodium methoxide (23.69 g, 110 mmol) was added, and the reaction mixture was stirred for 4 h at RT. The reaction mixture was filtered, and the filtrate was acidified with AcOH. The MeOH was evaporated, and the resulting precipitate was collected by filtration and suspended in dioxane (10 mL). Sodium hydroxide (1.896 mL, 9.48 mmol) was added, and the reaction mixture was stirred at 80° C. for 4 h. After cooling, the reaction mixture was neutralized with HCl and the organic phases evaporated to dryness. The product was collected and washed with water to give 4-((5-amino-7-hydroxy-1H-pyrazolo[4,3-d]pyrimidin-1-yl)methyl)-3-methoxybenzoic acid (250 mg, 0.793 mmol, 13% yield) as a solid.

LC-MS (ES, m/z): [M+H]⁺ 316.1.

Step 7. A 20 mL scintillation vial was charged with 4-((5-amino-7-hydroxy-1H-pyrazolo[4,3-d]pyrimidin-1-yl)methyl)-3-methoxybenzoic acid (250 mg, 0.793 mmol), HATU (332 mg, 0.872 mmol), DIPEA (0.277 mL, 1.586 mmol) and DMF (5 mL). The reaction mixture was stirred overnight at 50° C., cooled, filtered and purified using reverse-phase flash chromatography (C₁₈ column, 0 to 30% acetonitrile in water containing 0.05% formic acid), giving 4-((5-amino-7-hydroxy-1H-pyrazolo[4,3-d]pyrimidin-1-yl)methyl)-3-methoxy-N-(1-methylpiperidin-4-yl)benzamide (230 mg, 0.559 mmol, 70.5% yield) as a solid.

LC-MS (ES, m/z): [M+H]⁺ 412.3.

¹H NMR (400 MHz, DMSO-d₆) δ 8.26-8.19 (m, 1H), 7.56 (s, 1H), 7.40 (d, J=1.3 Hz, 1H), 7.31-7.27 (m, 1H), 6.52 (d, J=7.9 Hz, 1H), 6.08 (s, 2H), 5.62 (s, 2H), 3.87 (s, 3H), 3.84-3.75 (m, 1H), 2.99 (br d, J=11.7 Hz, 2H), 2.36 (s, 4H), 1.82 (br d, J=10.3 Hz, 2H), 1.75-1.54 (m, 2H).

Step 8. A 20 mL scintillation vial was charged with 4-((5-amino-7-hydroxy-1H-pyrazolo[4,3-d]pyrimidin-1-yl)methyl)-3-methoxy-N-(1-methylpiperidin-4-yl)benzamide (200 mg, 0.486 mmol), (3-(2-chloroethyl)cyclobutyl)methanamine hydrochloride (224 mg, 1.215 mmol), BOP (322 mg, 0.729 mmol), DBU (0.220 mL, 1.458 mmol) and DMSO (5 mL). The reaction mixture was stirred at RT for 2 h, diluted with water (2 mL), filtered and purified using reverse-phase flash chromatography (C₁₈ column, 0 to 75% MeCN in water containing 10 mM TEAA) gave 4-((5-amino-7-(((3-(2-chloroethyl)cyclobutyl)methyl)amino)-1H-pyrazolo[4,3-d]pyrimidin-1-yl)methyl)-3-methoxy-N-(1-methylpiperidin-4-yl)benzamide (227 mg, 0.420 mmol, 86% yield) as a brown solid.

LC-MS (ES, m/z): [M+H]⁺ 541.4.

Step 9. A 20 mL scintillation vial was charged with 4-((5-amino-7-(((3-(2-chloroethyl)cyclobutyl)methyl)amino)-1H-pyrazolo[4,3-d]pyrimidin-1-yl)methyl)-3-methoxy-N-(1-methylpiperidin-4-yl)benzamide (150 mg, 0.277 mmol), sodium acetate (227 mg, 2.77 mmol) and DMF (3 mL). The reaction mixture was stirred at 100° C. for 4 days, cooled, and diluted with water (3 mL). Purification using reverse-phase flash chromatography (C₁₈ column, 0 to 65% acetonitrile in water containing 0.05% TFA) afforded an intermediate product. The intermediate product was dissolved in EtOH (5 mL) and 10% Pd/C (10 mg) was added. The reaction vessel was evacuated and purged six times with hydrogen. Its contents were then stirred for 2 h under a hydrogen atmosphere. The reaction mixture was filtered and evaporated to dryness. The crude product was purified via preparative LC/MS with the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-minute hold at 11% B, 11-51% B over 25 min, then a 0-minute hold at 100% B; Flow Rate: 20 mL/min; Column Temperature: 25° C. Fraction collection was triggered by MS signals. Fractions containing the desired product were combined and dried via centrifugal evaporation, giving Compound 124 (42.6 mg, 0.084 mmol, 30% yield).

### Example 3—Compound 121

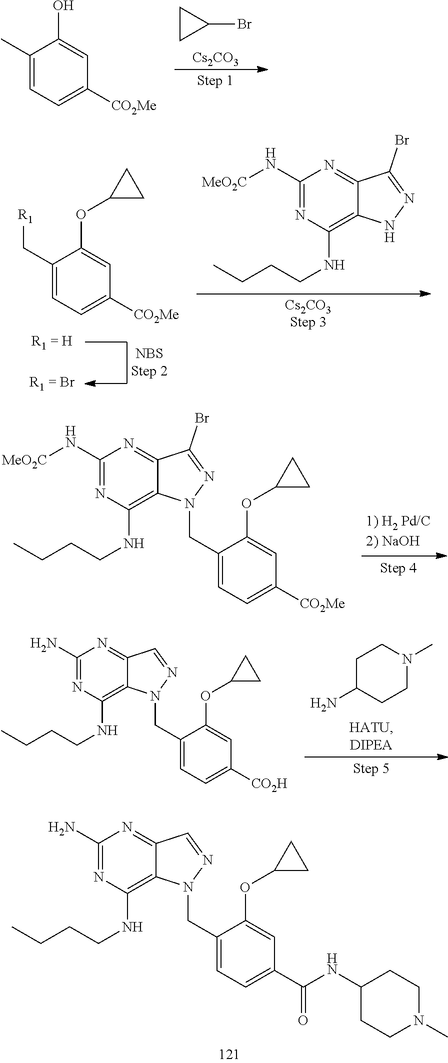

Step 1. A microwave vial was charged with methyl 3-hydroxy-4-methylbenzoate (2 g, 12.04 mmol), bromocyclopropane (1.747 g, 14.44 mmol), Cs₂CO₃ (4.71 g, 14.44 mmol) and DMF (15 mL). The reaction mixture was heated in a microwave oven at 160° C. for 3 h. After cooling, the reaction mixture was poured into water (150 mL) and extracted with EtOAc (3×50 mL). The combined organic phases were washed with brine (4×50 mL), dried (MgSO₄), filtered and concentrated. Flash chromatography (SiO₂ column, 0 to 5% EtOAc in hexanes) gave methyl 3-cyclopropoxy-4-methylbenzoate (980 mg, 1.901 mmol, 15.79% yield, purity 40%) as an oil, which was used in the next step without further purification.

LC-MS (ES, m/z): [M+H]⁺ 207.1.

Step 2. Methyl 3-cyclopropoxy-4-methylbenzoate (1 g, 1.939 mmol, 40% pure) was dissolved in CCl₄ (5 mL). NBS (0.759 g, 4.27 mmol) and benzoyl peroxide (0.103 g, 0.427 mmol) were added. The reaction mixture was stirred overnight at 70° C. After cooling, the reaction mixture was evaporated to dryness. Flash chromatography (SiO₂ column, 0 to 10% EtOAc in hexanes) gave methyl 4-(bromomethyl)-3-cyclopropoxybenzoate (550 mg, 1.54 mmol, purity 80%, 80% yield) as a solid. The product was used in the next step without further purification.

LC-MS (ES, m/z): [M+H]⁺ 285.0, 287.0.

Step 3. To a stirred solution of methyl (3-bromo-7-(butylamino)-1H-pyrazolo[4,3-d]pyrimidin-5-yl)carbamate (650 mg, 1.894 mmol; US 2020/0038403 A1) in DMF (5 mL) at 0° C. was added Cs₂CO₃ (1296 mg, 3.98 mmol), followed by a solution of methyl 4-(bromomethyl)-3-cyclopropoxybenzoate (540 mg, 1.515 mmol, 80% pure) in DMF (2 mL). The reaction mixture was allowed to warm to RT, was stirred overnight, poured into saturated NaHCO₃ solution (100 mL), and extracted with EtOAc (3×50 mL). The combined organic phases were washed with brine (4×50 mL), dried (MgSO₄), filtered and concentrated. Flash chromatography (SiO₂ column, 0 to 70% EtOAc in hexanes) gave methyl 4-((3-bromo-7-(butylamino)-5-((methoxy-carbonyl)amino)-1H-pyrazolo[4,3-d]pyrimidin-1-yl)methyl)-3-cyclopropoxybenzoate (153 mg, 0.279 mmol, 14.76% yield) as an oil which solidified on standing.

LC-MS (ES, m/z): [M+H]⁺ 547.2, 549.2.

¹H NMR (400 MHz, DMSO-d₆) δ 9.86 (s, 1H), 7.80 (d, J=1.5 Hz, 1H), 7.53 (dd, J=7.9, 1.5 Hz, 1H), 7.32 (t, J=5.5 Hz, 1H), 6.91 (d, J=7.9 Hz, 1H), 5.72 (s, 2H), 4.03-3.93 (m, 1H), 3.85 (s, 3H), 3.71-3.60 (m, 3H), 3.56-3.45 (m, 2H), 1.56 (quin, J=7.3 Hz, 2H), 1.22 (dq, J=14.8, 7.4 Hz, 2H), 0.85 (t, J=7.4 Hz, 3H), 0.81-0.73 (m, 2H), 0.52-0.41 (m, 2H).

Step 4. Methyl 4-((3-bromo-7-(butylamino)-5-((methoxycarbonyl)amino)-1H-pyrazolo[4,3-d]pyrimidin-1-yl)methyl)-3-cyclopropoxybenzoate (150 mg, 0.274 mmol) was dissolved in EtOH (5 mL). 10% Pd/C (15 mg) was added. The reaction vessel was evacuated and purged with hydrogen six times. The reaction mixture stirred under a hydrogen atmosphere for 1 h. The reaction mixture was filtered and the filtrate evaporated to dryness. The residue was dissolved in dioxane (3 mL), and sodium hydroxide (822 μl, 4.11 mmol) was added. The reaction mixture was stirred at 80° C. for 2 h, cooled, acidified with 5N HCl, and diluted with water (5 mL). The organic solvents were evaporated off, and the aqueous residue was purified using reverse-phase flash chromatography (C₁₈ column, 0 to 70% MeCN in water containing 0.05% TFA) gave 4-((5-amino-7-(butylamino)-1H-pyrazolo[4,3-d]pyrimidin-1-yl)methyl)-3-cyclopropoxybenzoic acid (35 mg, 0.088 mmol, 32% yield) as a solid.

LC-MS (ES, m/z): [M+H]⁺ 397.2.

¹H NMR (400 MHz, DMSO-d₆) δ 8.21 (br t, J=5.6 Hz, 1H), 7.81 (br s, 2H), 7.73 (s, 1H), 7.69 (s, 1H), 7.42 (dd, J=7.9, 1.3 Hz, 1H), 6.81 (d, J=7.9 Hz, 1H), 5.66 (s, 2H), 3.87 (tt, J=5.9, 2.9 Hz, 1H), 3.48 (q, J=6.7 Hz, 2H), 1.48 (quin, J=7.3 Hz, 2H), 1.14 (sxt, J=7.4 Hz, 2H), 0.78 (t, J=7.4 Hz, 3H), 0.75-0.68 (m, 2H), 0.48-0.38 (m, 2H).

Step 5. A 20 mL scintillation vial was charged with 4-((5-amino-7-(butylamino)-1H-pyrazolo[4,3-d]pyrimidin-1-yl)methyl)-3-cyclopropoxybenzoic acid (35 mg, 0.088 mmol), HATU (40.3 mg, 0.106 mmol), 1-methylpiperidin-4-amine (20.16 mg, 0.177 mmol) and DMF (2 mL). DIPEA (0.046 mL, 0.265 mmol) was added. The reaction mixture stirred at RT for 1 h. The reaction mixture was filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 2% B, 2-42% B over 20 min, then a 0-minute hold at 100% B; Flow Rate: 20 mL/min; Column Temperature: 25° C. Fraction collection was triggered by MS signals. Fractions containing the desired product were combined and dried via centrifugal evaporation, giving Compound 121 (31.2 mg, 0.063 mmol, 72% yield).

Compound 122 was analogously prepared.

### Example 4—Compound 120

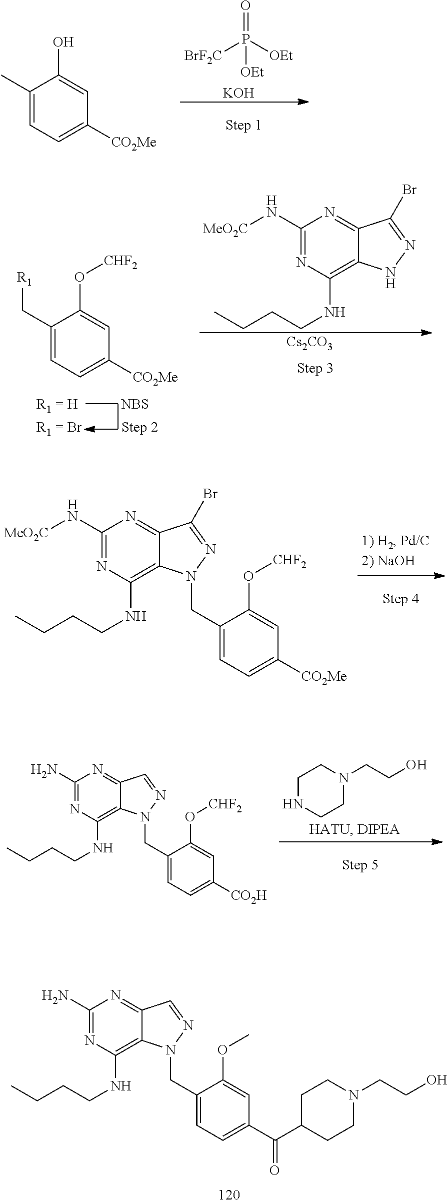

Step 1. A solution of KOH (5N, 24.07 mL, 120 mmol) in water was added to a cooled (ice bath) solution of methyl 3-hydroxy-4-methylbenzoate (4 g, 24.07 mmol) in acetonitrile (150 mL). After stirring at 0° C. for 5 min, diethyl (bromodifluoromethyl)phosphonate (12.85 g, 48.1 mmol) was added. The reaction mixture was allowed to warm slowly to RT and stirred for 16 h. More KOH solution (5N, 16 mL, 80 mmol) was added. The reaction mixture was stirred at RT for a further 30 min, diluted with water (200 mL), and extracted with EtOAc (3×50 mL). The combined organic phases washed with brine (2×50 mL), dried (MgSO₄), filtered and concentrated. Flash chromatography (SiO₂ column, 0 to 10% EtOAc in hexanes) gave methyl 3-(difluoromethoxy)-4-methylbenzoate (2.552 g, 11.80 mmol, 49.0% yield) as an oil.

LC-MS (ES, m/z): [M+H]⁺ 217.1.

¹H NMR (400 MHz, DMSO-d₆) δ 7.76 (dd, J=7.8, 1.7 Hz, 1H), 7.68 (br. s, 1H), 7.51-7.10 (m, 2H), 3.87 (s, 3H), 2.31 (s, 3H).

Step 2. NBS (1.811 g, 10.18 mmol) and benzoyl peroxide (0.448 g, 1.850 mmol) were added to a stirred solution of methyl 3-(difluoromethoxy)-4-methylbenzoate (2 g, 9.25 mmol) in carbon tetrachloride (20 mL) The reaction was stirred at 75° C. for 4 h, then at RT overnight. The reaction mixture was evaporated to dryness and purified using flash chromatography (SiO2 column, 0 to 15% EtOAc in hexanes), giving methyl 4-(bromomethyl)-3-(difluoromethoxy)-benzoate (1.561 g, 5.29 mmol, 57.2% yield) as an oil.

LC-MS (ES, m/z): [M+H]⁺ 295.0, 297.0.

¹H NMR (400 MHz, CDCl₃) δ 7.88 (dd, J=8.1, 1.5 Hz, 1H), 7.80 (s, 1H), 7.52 (d, J=8.1 Hz, 1H), 6.64 (t, J=73.0 Hz, 1H), 4.57-4.51 (m, 2H), 3.98-3.90 (m, 3H).

Step 3. Cs₂CO₃ (1329 mg, 4.08 mmol) was added to a stirred solution of methyl (3-bromo-7-(butylamino)-1H-pyrazolo[4,3-d]pyrimidin-5-yl)carbamate (700 mg, 2.040 mmol) in DMF (5 mL). After cooling in an ice bath, a solution of methyl 4-(bromomethyl)-3-(difluoro-methoxy)benzoate (572 mg, 1.938 mmol) in DMF (2 mL) was added. The reaction mixture was allowed to warm to RT and stirred for 3 h. Water (20 mL) was added, and the reaction mixture extracted with EtOAc (3×5 mL). The combined organic phases were washed with brine (4×10 mL), dried (MgSO₄), filtered and concentrated. Flash chromatography (SiO₂ column, loaded in DCM, 0 to 60% EtOAc in hexanes) gave methyl 4-((3-bromo-7-(butylamino)-5-((methoxy-carbonyl)amino)-1H-pyrazolo[4,3-d]pyrimidin-1-yl)methyl)-3-(difluoromethoxy)benzoate (275 mg, 0.493 mmol, 24.19% yield) as a solid.

LC-MS (ES, m/z): [M+H]⁺ 557.1, 559.1.

¹H NMR (400 MHz, DMSO-d₆) δ 9.89 (s, 1H), 7.82-7.69 (m, 2H), 7.61-7.14 (m, 2H), 6.87 (d, J=7.9 Hz, 1H), 5.88 (s, 2H), 3.87 (s, 3H), 3.64 (s, 3H), 3.54-3.45 (m, 2H), 1.58-1.46 (m, 2H), 1.19 (dq, J=15.0, 7.4 Hz, 2H), 0.83 (t, J=7.3 Hz, 3H).

Step 4. Methyl 4-((3-bromo-7-(butylamino)-5-((methoxycarbonyl)amino)-1H-pyrazolo[4,3-d]pyrimidin-1-yl)methyl)-3-(difluoromethoxy)benzoate (275 mg, 0.493 mmol) was dissolved in EtOH (15 mL). 10% Pd/C (27 mg) was added. The reaction vessel was evacuated and purged six times, with hydrogen and then stirred under a hydrogen atmosphere for 2 h. The reaction mixture was filtered and evaporated to dryness. The residue was dissolved in dioxane (2 mL). Sodium hydroxide (0.564 mL, 2.82 mmol) was added, and the reaction mixture was stirred at 80° C. for 2 h, and then allowed to cool. The reaction mixture was neutralized with 5N HCl and evaporated to dryness. The residue was dissolved in MeOH/water (1:1, 8 mL). The methanol was removed by evaporation. The residual aqueous suspension filtered, washing with water, to give 4-((5-amino-7-(butylamino)-1H-pyrazolo[4,3-d]pyrimidin-1-yl)methyl)-3-(difluoromethoxy)benzoic acid (54 mg, 0.133 mmol, 27% yield) as a solid.

LC-MS (ES, m/z): [M+H]⁺=407.2.

¹H NMR (400 MHz, DMSO-d₆) δ 8.50 (br s, 1H), 7.84 (s, 2H), 7.79-7.68 (m, 2H), 7.63-7.05 (t, J=73.2 Hz 1H), 6.97 (d, J=7.9 Hz, 1H), 5.94 (s, 2H), 3.54 (q, J=6.4 Hz, 2H), 1.54 (quin, J=7.2 Hz, 2H), 1.19 (d q, J=14.9, 7.3 Hz, 2H), 0.84 (t, J=7.3 Hz, 3H).

Step 5. A 20 mL scintillation vial was charged with 4-((5-amino-7-(butylamino)-1H-pyrazolo[4,3-d]pyrimidin-1-yl)methyl)-3-(difluoromethoxy)benzoic acid (30 mg, 0.074 mmol), HATU (33.7 mg, 0.089 mmol), 2-(piperazin-1-yl)ethan-1-ol (9.61 mg, 0.074 mmol) and DMF (2 mL). DIPEA (0.039 mL, 0.221 mmol) was added. The reaction stirred at RT for 1 h, filtered and purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 5% B, 5-45% B over 20 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 120, 2TFA salt (28.1 mg, 0.037 mmol, 51% yield).

### Example 5—Compound 117

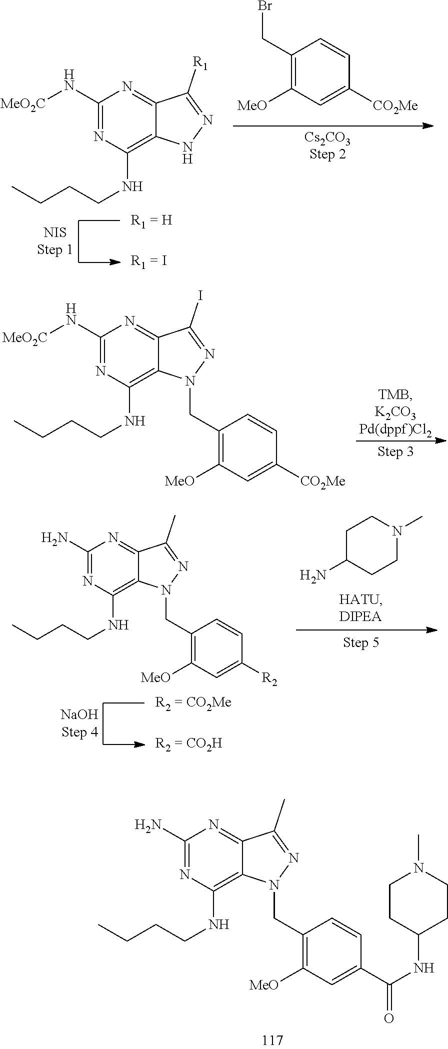

Step 1. A stirred solution of methyl (7-(butylamino)-1H-pyrazolo[4,3-d]pyrimidin-5-yl)carbamate (4.98 g, 18.84 mmol; US 2020/0038403 A1) in DMF (60 mL) was cooled with an ice bath. NIS (5.09 g, 22.61 mmol) was added portion-wise. The reaction mixture stirred at RT for 2 h and poured into water (400 mL). The product was collected by filtration, giving methyl (7-(butylamino)-3-iodo-1H-pyrazolo[4,3-d]pyrimidin-5-yl)carbamate (6.46 g, 15.73 mmol, 83% yield) as a solid.

LC-MS (ES, m/z): [M+H]⁺=391.1.

¹H NMR (400 MHz, DMSO-d6) δ 12.96 (s, 1H), 9.74 (s, 1H), 7.52 (s, 1H), 3.62 (s, 3H), 3.53 (q, J=6.5 Hz, 2H), 1.68-1.55 (m, 2H), 1.40 (m, 2H), 0.94 (t, J=7.4 Hz, 3H).

Step 2. Cs₂CO₃ (4.18 g, 12.81 mmol) was added to a stirred solution of methyl (7-(butylamino)-3-iodo-1H-pyrazolo[4,3-d]pyrimidin-5-yl)carbamate (2.5 g, 6.41 mmol) in DMF (50 mL). After sonicating for 5 min, a solution of methyl 4-(bromomethyl)-3-methoxybenzoate (1.743 g, 6.73 mmol) in DMF (10 mL) was added. The reaction mixture was stirred at RT for 2 h, poured into 10% citric acid solution (100 mL), and extracted with DCM (3×100 mL). The combined organic phases were washed with brine, dried (Na₂SO₄), filtered and concentrated. Flash chromatography (SiO₂ column, 0 to 100% EtOAc in hexanes) gave methyl 4-((7-(butylamino)-3-iodo-5-((methoxycarbonyl)amino)-1H-pyrazolo[4,3-d]pyrimidin-1-yl)methyl)-3-methoxybenzoate (2.26 g, 2.98 mmol, 46% yield, 75% purity) as a solid, which was used in the next step without further purification.

LC-MS (ES, m/z): [M+H]⁺=569.2.

Step 3. A 20 mL microwave vial was charged with methyl 4-((7-(butylamino)-3-iodo-5-((methoxycarbonyl)amino)-1H-pyrazolo[4,3-d]pyrimidin-1-yl)methyl)-3-methoxybenzoate (1.34 g, 1.771 mmol, 75% purity), [1,1′-bis(diphenylphosphino)ferrocene]dichloropalladium(II) (Pd(dppf)Cl₂, 91 mg, 0.124 mmol), trimethylboroxine (TMB, 1001 mg, 7.97 mmol), K₂CO₃ (734 mg, 5.31 mmol) and dioxane (7 mL). The reaction mixture was heated in a microwave oven at 120° C. for 1 h and diluted with DCM and 10% citric acid. The phases were separated. The organic phase was washed sequentially with 10% citric acid and brine, dried (Na₂SO₄) filtered and concentrated under reduced pressure. Flash chromatography give methyl 4-((5-amino-7-(butylamino)-3-methyl-1H-pyrazolo[4,3-d]pyrimidin-1-yl)methyl)-3-methoxybenzoate (422 mg, 1.06 mmol, 59.8% yield) as a solid.

LC-MS (ES, m/z): [M+H]⁺=399.2.

Step 4. NaOH (1.190 mL, 5.95 mmol) was added to a suspension of methyl 4-((5-amino-7-(butylamino)-3-methyl-1H-pyrazolo[4,3-d]pyrimidin-1-yl)methyl)-3-methoxybenzoate (237 mg, 0.595 mmol) in dioxane (5 mL). The reaction mixture stirred at 80° C. for 1 h, cooled, s neutralized with 5N hydrochloric acid, and evaporated to dryness. The residue was suspended in DMSO (2 mL) and water (20 mL) and filtered off, washing with water, giving 4-((5-amino-7-(butylamino)-3-methyl-1H-pyrazolo[4,3-d]pyrimidin-1-yl)methyl)-3-methoxybenzoic acid (184 mg, 0.479 mmol, 80% yield) as a solid.

LC-MS (ES, m/z): [M−H]⁺=383.2.

Step 5. A 20 mL scintillation vial was charged with 4-((5-amino-7-(butylamino)-3-methyl-1H-pyrazolo[4,3-d]pyrimidin-1-yl)methyl)-3-methoxybenzoic acid (35 mg, 0.091 mmol), HBTU (41.4 mg, 0.109 mmol), 1-methylpiperidin-4-amine (20.79 mg, 0.182 mmol) and DMF (2 mL). DIPEA (0.048 mL, 0.273 mmol) was added. The reaction was stirred at RT overnight. The reaction mixture was filtered and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 3% B, 3-43% B over 20 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 117 (37.6 mg, 0.78 mmol, 85%).

Compound 118 and Compound 119 were analogously prepared.

### Example 6-Compound 126, TFA Salt

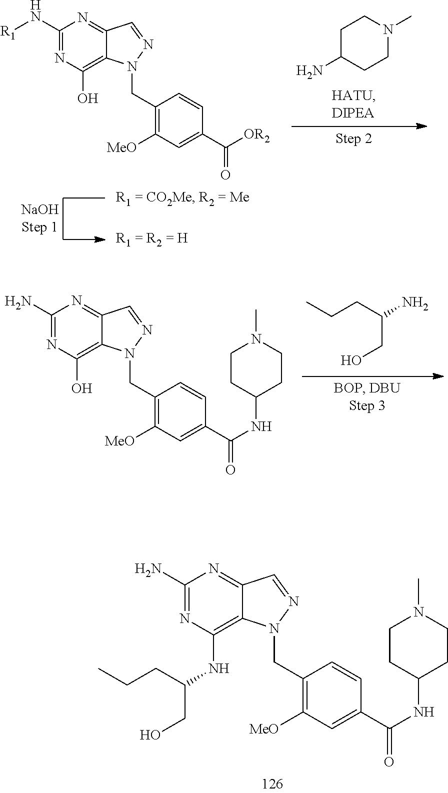

Step 1: NaOH (651 mg, 16.26 mmol) was added to a suspension of methyl 3-methoxy-4-((5-((methoxycarbonyl)amino)-7-oxo-6,7-dihydro-1H-pyrazolo[4,3-d]pyrimidin-1-yl)methyl)benzoate (630 mg, 1.626 mmol; US 2020/0038403 A1) in dioxane (16 mL) and water (3.3 mL). The reaction mixture was heated at 80° C. for 2 h and cooled. The dioxane was evaporated. The reaction mixture was diluted with water and acidified to pH 4 with concentrated HCl. The precipitate was filtered off, washed with water, and dried. The material was azeotroped with toluene and dried further to give 4-((5-amino-7-oxo-6,7-dihydro-1H-pyrazolo-[4,3-d]pyrimidin-1-yl)methyl)-3-methoxybenzoic acid (500 mg, 1.586 mmol, 98% yield).

LC/MS (Method F): RT=0.50 min. M/Z=316.0.

Step 2: Hunig's base (0.277 mL, 1.586 mmol) and HATU (332 mg, 0.872 mmol) were added to a suspension of 1-methylpiperidin-4-amine (136 mg, 1.189 mmol) and 4-((5-amino-7-oxo-6,7-dihydro-1H-pyrazolo[4,3-d]pyrimidin-1-yl)methyl)-3-methoxybenzoic acid (250 mg, 0.793 mmol) in DMF (5 mL). The reaction mixture was heated to 50° C. After 4.5 h, 1-methylpiperidin-4-amine (136 mg, 1.189 mmol) and 160 mg HATU were added and the reaction mixture was heated overnight. 200 mg piperidine, HATU (332 mg, 0.872 mmol), and Hunig's base (0.277 mL, 1.586 mmol) were added, and heating was continued for 5 h. 1-methylpiperidin-4-amine (136 mg, 1.189 mmol) and HATU (332 mg, 0.872 mmol) were added and heating was continued overnight. 1-methylpiperidin-4-amine (136 mg, 1.189 mmol) and 165 mg HATU were added. After 5 h, the reaction mixture was cooled and diluted with water. The solid was collected by filtration, washed with water, and dried to give 4-((5-amino-7-oxo-6,7-dihydro-1H-pyrazolo[4,3-d]pyrimidin-1-yl)methyl)-3-methoxy-N-(1-methylpiperidin-4-yl)benzamide (126 mg, 0.306 mmol, 38.6% yield).

¹H NMR (400 MHz, DMSO-d₆) δ 8.16 (d, J=7.6 Hz, 1H), 7.57 (s, 1H), 7.41 (d, J=1.3 Hz, 1H), 7.32-7.26 (m, 1H), 6.52 (d, J=8.0 Hz, 1H), 6.05 (br d, J=1.8 Hz, 2H), 5.63 (s, 2H), 3.88 (s, 3H), 3.74-3.63 (m, 1H), 2.80-2.71 (m, 2H), 2.15 (s, 3H), 1.97-1.86 (m, 2H), 1.79-1.68 (m, 2H), 1.62-1.49 (m, 2H).

LC/MS (Method A): RT=0.52 min. M/Z=412.4.

Step 3. DBU (0.022 mL, 0.146 mmol) and BOP (48.4 mg, 0.109 mmol) were added to a suspension of (S)-2-aminopentan-1-ol (37.6 mg, 0.365 mmol) and 4-((5-amino-7-oxo-6,7-dihydro-1H-pyrazolo[4,3-d]pyrimidin-1-yl)methyl)-3-methoxy-N-(1-methylpiperidin-4-yl)benzamide (30 mg, 0.073 mmol) in DMF (0.5 mL). All the suspended material dissolved in minutes. After 1.75 h, the reaction mixture was cooled, diluted with MeOH, and filtered through a syringe filter. The crude material was purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-minute hold at 5% B, 5-45% B over 25 min, then a 0-minute hold at 100% B; Flow Rate: 20 mL/min; Column Temperature: 25° C. Fraction collection was triggered by MS and UV signals. Fractions containing the desired product were combined and dried via centrifugal evaporation to give Compound 126, as its TFA salt (16 mg, 0.025 mmol, 34.2% yield).

### Example 7-Compound 123

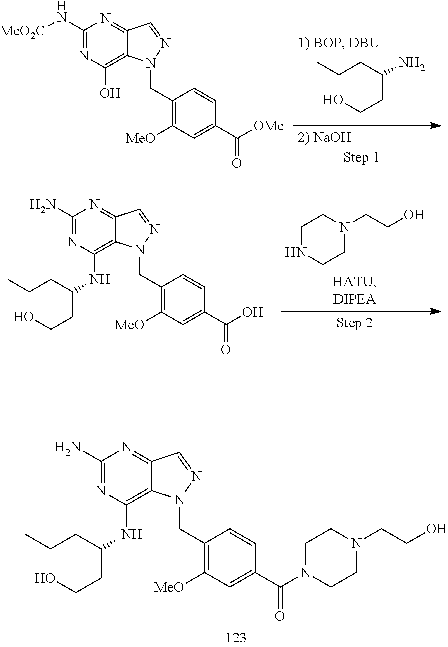

Step 1. DBU (0.856 mL, 5.68 mmol) was added to a suspension of methyl 4-((7-hydroxy-5-((methoxycarbonyl)amino)-1H-pyrazolo[4,3-d]pyrimidin-1-yl)methyl)-3-methoxy-benzoate (550 mg, 1.420 mmol; see Step 6 of Example 2 before NaOH treatment) and (S)-3-aminohexan-1-ol hydrochloride 2 (327 mg, 2.130 mmol) in DMSO (5 mL). The reaction mixture was stirred at RT for 10 min, when it became a clear solution. BOP (1256 mg, 2.84 mmol) was added. The reaction mixture was stirred at 70° C. for 2 h. 5M NaOH (5 mL, 25.00 mmol) was added and the reaction mixture was stirred at 70° C. for 0.5 h. After cooling, it was filtered through a syringe filter disc. The filtrate was purified on preparative reverse C18 column (150 g), eluted with acetonitrile:water (with 0.05% TFA modifier), 0-50% gradient. The desired fraction was frozen and lyophilized to afford (S)-4-((5-amino-7-((1-hydroxyhexan-3-yl)amino)-1H-pyrazolo[4,3-d]pyrimidin-1-yl)methyl)-3-methoxybenzoic acid (860.8 mg, 1.246 mmol, 88% yield).

LCMS ESI: calculated for C₂₀H₂₇N₆O₄=415.2 (M+H⁺), found 415.2(M+H⁺).

Step 2. A mixture of (S)-4-((5-amino-7-((1-hydroxyhexan-3-yl)amino)-1H-pyrazolo[4,3-d]pyrimidin-1-yl)methyl)-3-methoxybenzoic acid (30 mg, 0.072 mmol), 2-(piperazin-1-yl)ethan-1-ol (18.85 mg, 0.145 mmol) in DMF (1 mL) was treated with Hunig's base (0.063 mL, 0.362 mmol), followed by BOP (48.0 mg, 0.109 mmol). The reaction mixture was stirred at RT for 3 h and filtered through a syringe frit disc. The filtrate was purified via LC/MS Method H. Fractions containing the desired product were combined and dried via centrifugal evaporation to yield Compound 123 (12.1 mg, 0.022 mmol, 30.1% yield).

Compound 125 was analogously prepared.

### Example 8—Compound 101

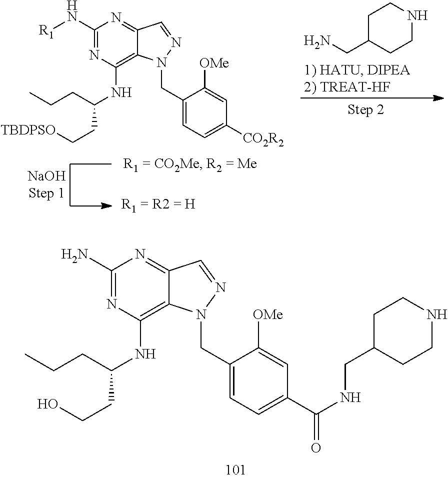

Step 1. A solution of methyl (S)-4-((7-((1-((tert-butyldiphenylsilyl)oxy)hexan-3-yl)amino)-5-((methoxycarbonyl)amino)-1H-pyrazolo[4,3-d]pyrimidin-1-yl)methyl)-3-methoxybenzoate (30 mg, 0.041 mmol; US 2020/0038403 A1) in dioxane (1 mL) was treated with NaOH (0.207 mL, 0.207 mmol) and heated at 80° C. for 2 h. The reaction mixture was neutralized to pH 7 by the slow addition of 6M aq. HCl. The solvent was evaporated in a V-10 apparatus and the residue was dissolved in DMF (2 mL) and filtered. The filtrate was purified on reverse phase ISCO (50 g column) eluting with 0-50% acetonitrile/water 90.05%) formic acid. The product containing fractions were lyophilized to provide the desired product as white solid. LCMS ESI: calculated for C₃₆H₄₄N₆O₄Si=653.8 (M+H⁺), found 653.4(M+H⁺).

Step 2. A solution of (S)-4-((5-amino-7-((1-((tert-butyldiphenylsilyl)oxy)hexan-3-yl)amino)-1H-pyrazolo[4,3-d]pyrimidin-1-yl)methyl)-3-methoxybenzoic acid (26 mg, 0.040 mmol) in DMF (1 mL) was treated with HATU (22.71 mg, 0.060 mmol) and stirred for 10 min, after which piperidin-4-ylmethanamine (4.55 mg, 0.040 mmol) was added. LCMS (M+H=749.6) after 30 min showed completion of the reaction. The solution was treated with triethylamine trihydrofluoride (0.065 mL, 0.398 mmol) and stirred for 2 h after which LCMS (M+H=511.3) showed loss of the TBDPS protecting group. Excess of triethylamine trihydrofluoride (0.065 mL, 0.398 mmol) was neutralized by sat. aq. NaHCO₃ solution (1 mL) and the solvents were evaporated in a V-10 apparatus. The crude material was purified via LC/MS Method I. Fractions containing the desired product were combined and dried via centrifugal evaporation to provide Compound 101 (6.7 mg, 32% yield).

### Example 9—Compound 127

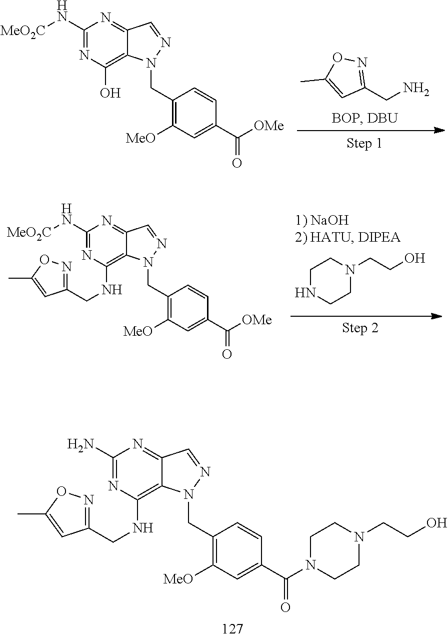

Step 1: A solution of methyl 4-((7-hydroxy-5-((methoxycarbonyl)amino)-1H-pyrazolo[4,3-d]pyrimidin-1-yl)methyl)-3-methoxybenzoate (US 2020/0038403 A1; 300 mg, 0.774 mmol) in DMSO (3.9 mL) was treated with (5-methylisoxazol-3-yl)methanamine (174 mg, 1.55 mmol), BOP (411 mg, 0.929 mmol) and DBU (233 μl, 1.549 mmol). The reaction mixture was stirred at RT for 2 h, diluted with EtOAc and washed with H₂O (3×). The organic layer was dried over Na₂SO₄, filtered and concentrated in vacuo to give methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (353 mg, 95% yield).

¹H NMR (400 MHz, DMSO-d₆) δ 9.80 (s, 1H), 7.99-7.93 (m, 1H), 7.77 (t, J=5.9 Hz, 1H), 7.49 (d, J=1.5 Hz, 1H), 7.45 (dd, J=7.8, 1.5 Hz, 1H), 6.62 (d, J=7.9 Hz, 1H), 6.10 (d, J=0.9 Hz, 1H), 5.80 (s, 2H), 4.73 (d, J=5.9 Hz, 2H), 3.84 (s, 3H), 3.82 (s, 3H), 3.64 (s, 3H), 2.31 (s, 3H).

LC RT: 0.67 min. LC/MS [M+H]⁺ 482.3 (Method J)

Step 2. A solution of methyl 3-methoxy-4-((5-((methoxycarbonyl)amino)-7-(((5-methylisoxazol-3-yl)methyl)amino)-1H-pyrazolo[4,3-d]pyrimidin-1-yl)methyl)benzoate (125 mg, 0.260 mmol) in dioxane (1.3 mL) was treated with NaOH (10 M aq. soln, 0.2 mL, 2.0 mmol) and heated to 75° C. After 2 h, the reaction mixture was cooled to RT and treated with HCl (4 M in dioxane, 0.52 mL, 2.1 mmol) and concentrated in vacuo. The residue was re-dissolved in MeOH/DCM and concentrated in vacuo. Of this crude material, 40 mg was dissolved in DMF (469 μl) and treated with 2-(piperazin-1-yl)ethan-1-ol (12 mg, 0.094 mmol), DIEA (41 μl, 0.23 mmol) and 2,4,6-tripropyl-1,3,5,2,4,6-trioxatriphosphorinane-2,4,6-trioxide (50% solution in EtOAc, 55.8 μL, 0.094 mmol). The reaction mixture was stirred at RT for 1 h, diluted with DMF (1 mL) and H₂O (0.2 mL), and filtered through a PTFE frit. The crude material was purified via preparative LC/MS with the following conditions: Column: XBridge C18, 200 mm×19 mm, 5-μm particles; Mobile Phase A: 5:95 acetonitrile: water with 0.05% TFA; Mobile Phase B: 95:5 acetonitrile: water with 0.05% TFA; Gradient: a 0-min hold at 0% B, 0-30% B over 20 minutes, then a 0-minute hold at 100% B; Flow Rate: 20 mL/min; Column Temperature: 25° C. Fraction collection was triggered by MS signals. Fractions containing product were combined and dried via centrifugal evaporation to give Compound 127 as the bis TFA salt. (11.7 mg, 39% yield).

Compound 129, Compound 130, and Compound 131 were analogously prepared.

### Example 10—Compound 128

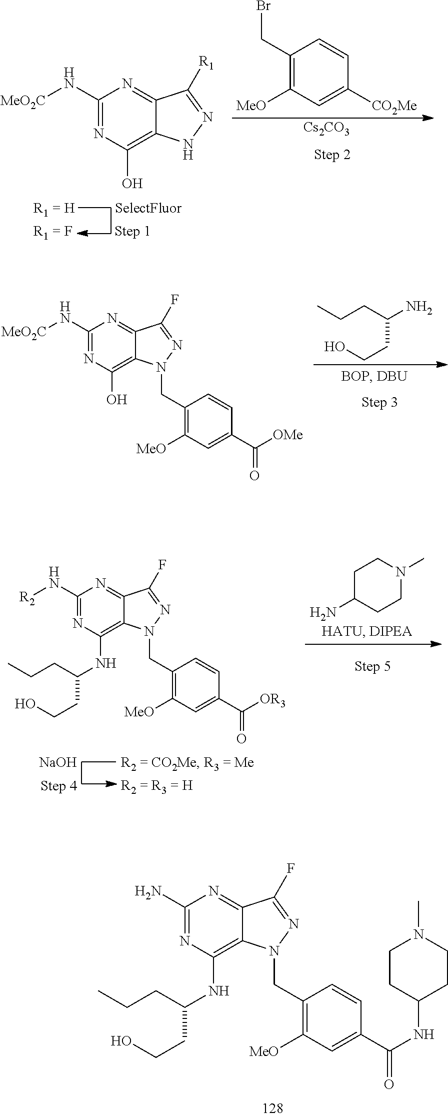

Step 1. Methyl (7-hydroxy-1H-pyrazolo[4,3-d]pyrimidin-5-yl)carbamate (1 g, 4.78 mmol) and Selectfluor™ (5.08 g, 14.34 mmol) were suspended in acetonitrile (20 mL). Acetic acid (2 mL) was added. The reaction mixture as stirred at 70° C. for 24 h, cooled, and poured into water (100 mL). The resulting mixture was cooled in a freezer (−20° C.) for 30 min, then the product was filtered off, washing with water (40 mL), giving methyl (3-fluoro-7-hydroxy-1H-pyrazolo[4,3-d]pyrimidin-5-yl)carbamate (623 mg, 2.74 mmol, 57.4% yield) as a solid.

LC-MS (ES, m/z): [M+H]⁺=228.2.

¹H NMR (400 MHz, DMSO-d₆) δ 13.69 (s, 1H), 11.63 (s, 1H), 11.26 (s, 1H), 3.76 (s, 3H).

Step 2. A stirred suspension of methyl (3-fluoro-7-hydroxy-1H-pyrazolo[4,3-d]pyrimidin-5-yl)carbamate (620 mg, 2.73 mmol) and Cs₂CO₃ (1030 mg, 3.16 mmol) in DMF (5 mL) was cooled in an ice bath. A solution of methyl 4-(bromomethyl)-3-methoxybenzoate (744 mg, 2.87 mmol) in DMF (5 mL) was added. The reaction allowed to warm slowly to RT, stirred for 2 h, poured into water (100 mL), and extracted with EtOAc (3×100 mL). The combined organic phases were washed with brine (4×50 mL), dried (MgSO₄), filtered and concentrated. Flash chromatography (40 g SiO₂ column, loaded on silica, 0 to 100% EtOAc in hexanes) gave methyl 4-((3-fluoro-7-hydroxy-5-((methoxycarbonyl)amino)-1H-pyrazolo[4,3-d]pyrimidin-1-yl)methyl)-3-methoxybenzoate (342 mg, ca. 60% pure, 0.506 mmol, 17.6% yield) as a solid. LC-MS (ES, m/z): [M+H]⁺=406.2.

Step 3. A 20 mL scintillation vial was charged with methyl 4-((3-fluoro-7-hydroxy-5-((methoxycarbonyl)amino)-1H-pyrazolo[4,3-d]pyrimidin-1-yl)methyl)-3-methoxybenzoate (340 mg, 0.839 mmol), (S)-3-aminohexan-1-ol, HCl (193 mg, 1.258 mmol), BOP (742 mg, 1.678 mmol), DMSO (4 mL) and DBU (0.379 mL, 2.52 mmol). The reaction mixture was heated at 70° C. for 15 min, cooled, poured into saturated NaHCO₃ solution (100 mL), and extracted with EtOAc (3×40 mL). The combined organic phases were washed with brine (4×40 mL), dried (MgSO₄), filtered and concentrated. Flash chromatography (40 g SiO₂ column, 0 to 100% EtOAc in hexanes) gave methyl (S)-4-((3-fluoro-7-((1-hydroxyhexan-3-yl)amino)-5-((methoxycarbonyl)-amino)-1H-pyrazolo[4,3-d]pyrimidin-1-yl)methyl)-3-methoxybenzoate (105 mg, 0.208 mmol, 24.8% yield) as a solid.

LC-MS (ES, m/z): [M+H]⁺=505.3.

¹H NMR (400 MHz, DMSO-d₆) δ 9.90 (s, 1H), 7.52 (s, 1H), 7.49 (d, J=8.2 Hz, 1H), 6.74 (d, J=7.7 Hz, 1H), 6.69 (d, J=7.9 Hz, 1H), 5.77 (d, J=17.2 Hz, 1H), 5.61 (d, J=16.9 Hz, 1H), 4.54-4.43 (m, 1H), 4.38 (t, J=5.5 Hz, 1H), 3.87 (s, 3H), 3.85 (s, 3H), 3.63 (s, 3H), 3.45-3.34 (m, 2H), 1.75-1.62 (m, 2H), 1.58-1.40 (m, 2H), 1.18-1.01 (m, 2H), 0.75 (t, J=7.4 Hz, 3H).

Step 4. Methyl (S)-4-((3-fluoro-7-((1-hydroxyhexan-3-yl)amino)-5-((methoxy-carbonyl)amino)-1H-pyrazolo[4,3-d]pyrimidin-1-yl)methyl)-3-methoxybenzoate (100 mg, 0.198 mmol) was dissolved in dioxane (2 mL) and sodium hydroxide (0.595 mL, 2.97 mmol) was added. The reaction mixture was stirred at 80° C. for 2 hours, then at RT overnight. The reaction mixture was neutralized using 5N HCl and evaporated to dryness, giving (S)-4-((5-amino-3-fluoro-7-((1-hydroxyhexan-3-yl)amino)-1H-pyrazolo[4,3-d]pyrimidin-1-yl)methyl)-3-methoxybenzoic acid (210 mg, ca. 40% pure, 0.19 mmol, 98% yield) which was then used without purification.

LC-MS (ES, m/z): [M+H]⁺=433.2.

Step 5. A 20 mL scintillation vial was charged with (S)-4-((5-amino-3-fluoro-7-((1-hydroxyhexan-3-yl)amino)-1H-pyrazolo[4,3-d]pyrimidin-1-yl)methyl)-3-methoxybenzoic acid (70 mg, 0.081 mmol), HATU (36.9 mg, 0.097 mmol) and DMF (2 mL). 1-methylpiperidin-4-amine (18.48 mg, 0.162 mmol) was added, followed by DIPEA (0.042 mL, 0.243 mmol). The reaction mixture stirred at RT for 1 h, filtered, and purified via preparative LC/MS with the following conditions: Column: XBridge C18, 200 mm×19 mm, 5-μm particles; Mobile Phase A: 5:95 acetonitrile: water with NH₄OAc; Mobile Phase B: 95:5 acetonitrile: water with NH₄OAc; Gradient: a 0-minute hold at 4% B, 4-44% B over 20 minutes, then a 0-minute hold at 100% B; Flow Rate: 20 mL/min; Column Temperature: 25° C. Fraction collection was triggered by MS and UV signals. Fractions containing the desired product were combined and dried via centrifugal evaporation, giving Compound 128 (15.1 mg, 0.028 mmol, 34.7% yield).

### Example 11—Compound 132

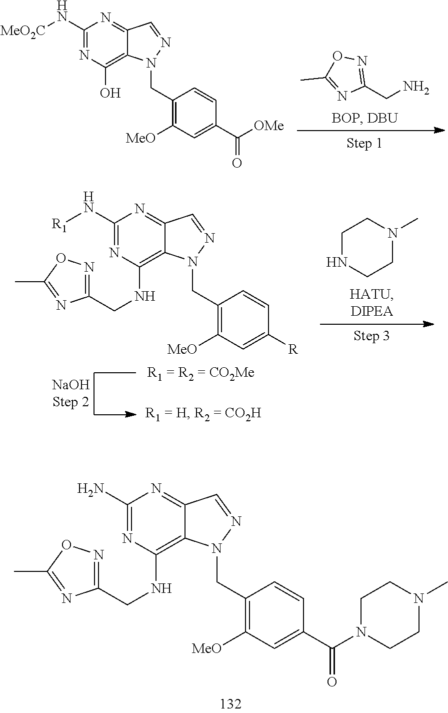

Step 1. A solution of methyl 4-((7-hydroxy-5-((methoxycarbonyl)amino)-1H-pyrazolo[4,3-d]pyrimidin-1-yl)methyl)-3-methoxybenzoate (510 mg, 1.32 mmol; US 2020/0038403 A1, FIG. 2A, compound 16) in DMSO (6.6 mL) was treated with (5-methyl-1,2,4-oxadiazol-3-yl)methanamine.HCl (236 mg, 1.58 mmol), BOP (698 mg, 1.58 mmol) and DBU (595 μL, 3.95 mmol). The reaction was stirred at RT. After 16 h, additional (5-methyl-1,2,4-oxadiazol-3-yl)methanamine.HCl (50 mg, 0.33 mmol), BOP (50 mg, 0.11 mmol) and DBU (200 μL, 1.33 mmol) were added. The reaction was stirred at RT for 2 h, diluted with EtOAc, and washed with H₂O (4×). The organic layer was absorbed onto Celite and purified via column chromatography (100 g C18 gold column; Mobile Phase A: 5:95 acetonitrile:water with 0.05% trifluoroacetic acid; Mobile Phase B: 95:5 acetonitrile:water with 0.05% trifluoroacetic acid; Flow Rate: 60 mL/min, 20-60% gradient). Fractions containing the desired product were combined, treated with HCl (1 M in H₂O, 2 mL, 2 mmol) and concentrated in vacuo to give methyl 3-methoxy-4-((5-((methoxycarbonyl)amino)-7-(((5-methyl-1,2,4-oxadiazol-3-yl)methyl)amino)-1H-pyrazolo[4,3-d]pyrimidin-1-yl)methyl)benzoate (382 mg, 60% yield). ¹H NMR (400 MHz, DMSO-d₆) δ 9.72-9.70 (m, 1H), 7.96-7.94 (m, 1H), 7.83-7.76 (m, 1H), 7.49 (d, J=1.4 Hz, 1H), 7.46 (dd, J=7.8, 1.5 Hz, 1H), 6.74 (d, J=7.8 Hz, 1H), 5.79 (s, 2H), 4.86 (d, J=5.8 Hz, 2H), 3.86 (s, 3H), 3.84 (s, 3H), 3.60 (s, 3H), 2.54 (s, 3H). LC RT: 0.64 min. LC/MS [M+H]⁺ 483.3 (Method J).

Step 2. A solution of methyl 3-methoxy-4-((5-((methoxycarbonyl)amino)-7-(((5-methyl-1,2,4-oxadiazol-3-yl)methyl)amino)-1H-pyrazolo[4,3-d]pyrimidin-1-yl)methyl)benzoate (382 mg, 0.791 mmol) in Dioxane (9.0 mL) was treated with NaOH (10 M aq. soln, 0.32 mL, 3.2 mmol) and heated to 40° C. After 30 minutes the temperature was increased to 60° C. Additional portions of NaOH (10M aqueous solution, 450 μL, 3 mmol) and MeOH (1 mL) were added to the reaction mixture over a period of 6 h. The reaction mixture was cooled to RT, neutralized with HOAc and concentrated in vacuo. The crude product was dissolved in MeOH, filtered through a PTFE frit, and purified via preparative HPLC with the following conditions: Column: Axia C18 100 mm×30 mm, 5-μm particles; Mobile Phase A: 10:90 Methanol: water with 0.1% trifluoroacetic acid; Mobile Phase B: 90:10 Methanol: water with 0.1% trifluoroacetic acid; Gradient: a 0-minute hold at 15% B, 15-30% B over 10 minutes, then a 4-minute hold at 30% B; Flow Rate: 40 mL/min; UV detection at 220 nm; Column Temperature: 25° C. Fractions containing the desired product were combined, treated with HCl (1 M in H₂O, 2 mL, 2 mmol) and concentrated in vacuo to give 4-((5-amino-7-(((5-methyl-1,2,4-oxadiazol-3-yl)methyl)amino)-1H-pyrazolo[4,3-d]pyrimidin-1-yl)methyl)-3-methoxybenzoic acid-HCl (98.9 mg, 28% yield). ¹H NMR (400 MHz, DMSO-d₆) δ 13.23-12.93 (m, 1H), 12.67-12.43 (m, 1H), 9.06-8.92 (m, 1H), 8.03-7.87 (m, 2H), 7.83 (s, 1H), 7.51-7.46 (m, 2H), 6.98 (d, J=8.2 Hz, 1H), 5.80 (s, 2H), 4.91 (d, J=5.7 Hz, 2H), 3.82 (s, 3H), 2.57 (s, 3H). LC RT: 0.52 min. LC/MS [M+H]⁺ 411.3 (Method J).

Step 3. A solution of 4-((5-amino-7-(((5-methyl-1,2,4-oxadiazol-3-yl)methyl)amino)-1H-pyrazolo[4,3-d]pyrimidin-1-yl)methyl)-3-methoxybenzoic acid-HCl (25 mg, 0.056 mmol) in DMF (0.6 mL) was treated with 1-methylpiperazine (11.2 mg, 0.112 mmol), DIEA (49 μL, 0.28 mmol) and 2,4,6-Tripropyl-1,3,5,2,4,6-trioxatriphosphorinane-2,4,6-trioxide (50% solution in EtOAc, 67 μL, 0.11 mmol). The reaction mixture was stirred at RT for 16 h and treated with additional 1-methylpiperazine (11.2 mg, 0.112 mmol), DIEA (49 μL, 0.28 mmol) and 2,4,6-tripropyl-1,3,5,2,4,6-trioxatriphosphorinane-2,4,6-trioxide (50% solution in EtOAc, 67 μL, 0.11 mmol) and stirred at RT overnight. The reaction mixture was diluted with DMF (1 mL) and H₂O (0.2 mL) and filtered through a PTFE frit. The crude material was purified via preparative LC/MS with the following conditions: Column: XBridge C18, 200 mm×19 mm, 5-μm particles; Mobile Phase A: 5:95 acetonitrile:water with 10 mM NH₄OAc; Mobile Phase B: 95:5 acetonitrile: water with 10 mM NH₄OAc; Gradient: a 0-minute hold at 0% B, 0-40% B over 20 minutes, then a 0-minute hold at 100% B; Flow Rate: 20 mL/min; Column Temperature: 25° C. Fraction collection was triggered by MS signals. Fractions containing the desired product were combined and dried via centrifugal evaporation to give Compound 132 (16.6 mg, 59% yield).

Compound 133 was analogously prepared.

### Example 12—Starting Materials and Intermediates

The Charts below show schemes for making compounds that could be useful as starting materials or intermediates for the preparation of TLR7 agonists disclosed herein. The schemes can be adapted to make other, analogous compounds that could be used as starting materials or intermediates. The reagents employed are well known in the art and in many instances their use has been demonstrated in the preceding Examples.

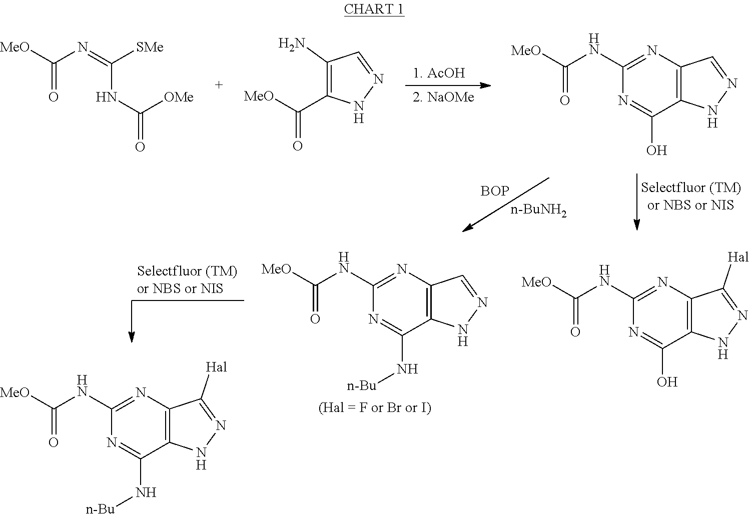

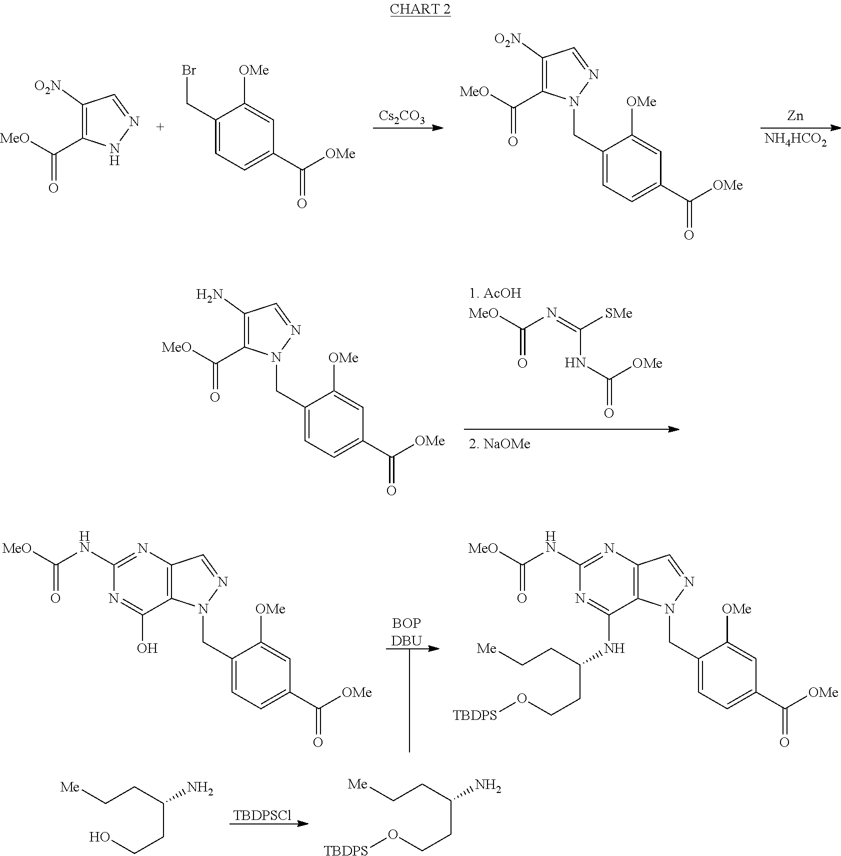

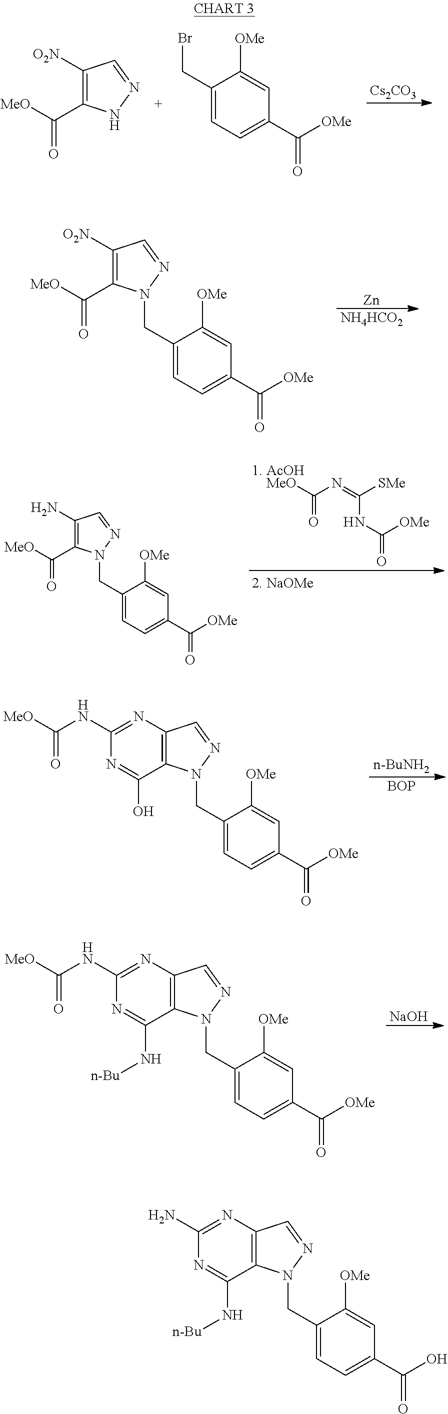

### Biological Activity

The biological activity of compounds disclosed herein as TLR7 agonists can be assayed by the procedures following.

### Human TLR7 Agonist Activity Assay

This procedure describes a method for assaying human TLR7 (hTLR7) agonist activity of the compounds disclosed in this specification.

Engineered human embryonic kidney blue cells (HEK-Blue™ TLR cells; Invivogen) possessing a human TLR7-secreted embryonic alkaline phosphatase (SEAP) reporter transgene were suspended in a non-selective, culture medium (DMEM high-glucose (Invitrogen), supplemented with 10% fetal bovine serum (Sigma)). HEK-Blue™ TLR7 cells were added to each well of a 384-well tissue-culture plate (15,000 cells per well) and incubated 16-18 h at 37° C., 5% CO₂. Compounds (100 nl) were dispensed into wells containing the HEK-Blue™ TLR cells and the treated cells were incubated at 37° C., 5% CO₂. After 18 h treatment ten microliters of freshly-prepared Quanti-Blue™ reagent (Invivogen) was added to each well, incubated for 30 min (37° C., 5% CO₂) and SEAP levels measured using an Envision plate reader (OD=620 nm). The half maximal effective concentration values (EC₅₀; compound concentration which induced a response halfway between the assay baseline and maximum) were calculated.

### Induction of Type I Interferon Genes (MX-1) and CD69 in Human Blood

The induction of Type I interferon (IFN) MX-1 genes and the B-cell activation marker CD69 are downstream events that occur upon activation of the TLR7 pathway. The following is a human whole blood assay that measures their induction in response to a TLR7 agonist.

Heparinized human whole blood was harvested from human subjects and treated with test TLR7 agonist compounds at 1 mM. The blood was diluted with RPMI 1640 media and Echo was used to predot 10 nL per well giving a final concentration of 1 uM (10 nL in 10 uL of blood). After mixing on a shaker for 30 sec, the plates were covered and placed in a 37° C. chamber for o/n=17 hrs. Fixing/lysis buffer was prepared (5×->1× in H₂O, warm at 37° C.; Cat #BD 558049) and kept the perm buffer (on ice) for later use.

For surface markers staining (CD69): prepared surface Abs: 0.045 ul hCD14-FITC (ThermoFisher Cat #MHCD1401)+0.6 ul hCD19-ef450 (ThermoFisher Cat #48-0198-42)+1.5 ul hCD69-PE (cat #BD555531)+0.855 ul FACS buffer. Added 3 ul/well, spin1000 rpm for 1 min and mixed on shaker for 30 sec, put on ice for 30 mins. Stop stimulation after 30 min with 70 uL of prewarmed 1× fix/lysis buffer and use Feliex mate to resuspend (15 times, change tips for each plate) and incubate at 37 C for 10 min.

Centrifuge at 2000 rpm for 5 min aspirate with HCS plate washer, mix on shaker for 30 sec and then wash with 70 uL in dPBS and pelleted 2×s (2000 rpm for 5 min) and 50 ul wash in FACS buffer pelleted 1×s (2000 rpm for 5 min). Mix on shaker for 30 sec. For Intracellular markers staining (MX-1): Add 50 ul of BD Perm buffer III and mix on shaker for 30 sec. Incubate on ice for 30 minutes (in the dark). Wash with 50 uL of FACS buffer 2× (spin @2300 rpm×5 min after perm) followed by mixing on shaker for 30 sec. Resuspended in 20 ul of FACS buffer containing MX1 antibody ( )(4812)-Alexa 647: Novus Biologicals #NBP2-43704AF647) 20 ul FACS bf+0.8 ul hlgG+0.04 ul MX-1. Spin 1000 rpm for 1 min, mix on shaker for 30se and the samples were incubated at RT in the dark for 45 minutes followed by washing 2×FACS buffer (spin @2300 rpm×5 min after perm). Resuspend 20 ul (35 uL total per well) of FACS buffer and cover with foil and place in 4° C. to read the following day. Plates were read on iQuePlus. The results were loaded into toolset and IC50 curves are generated in curve master. The y-axis 100% is set to 1 uM of resiquimod.

### Induction of TNF-Alpha and Type I IFN Response Genes in Mouse Blood

The induction of TNF-alpha and Type I IFN response genes are downstream events that occur upon activation of the TLR7 pathway. The following is an assay that measures their induction in whole mouse blood in response to a TLR7 agonist.

Heparinized mouse whole blood was diluted with RPMI 1640 media with Pen-Strep in the ratio of 5:4 (50 uL whole blood and 40 uL of media). A volume of 90 uL of the diluted blood was transferred to wells of Falcon flat bottom 96-well tissue culture plates, and the plates were incubated at 4° C. for 1 h. Test compounds in 100% DMSO stocks were diluted 20-fold in the same media for concentration response assays, and then 10 uL of the diluted test compounds were added to the wells, so that the final DMSO concentration was 0.5%. Control wells received 10 uL media containing 5% DMSO. The plates were then incubated at 37° C. in a 5% CO₂ incubator for 17 h. Following the incubation, 100 uL of the culture medium as added to each well. The plates were centrifuged and 130 uL of supernatant was removed for use in assays of TNFa production by ELISA (Invitrogen, Catalog Number 88-7324 by Thermo-Fisher Scientific). A 70 uL volume of mRNA catcher lysis buffer (1×) with DTT from the Invitrogen mRNA Catcher Plus kit (Cat #K1570-02) was added to the remaining 70 uL sample in the well, and was mixed by pipetting up and down 5 times. The plate was then shaken at RT for 5-10 min, followed by addition of 2 uL of proteinase K (20 mg/mL) to each well. Plates were then shaken for 15-20 min at RT. The plates were then stored at −80° C. until further processing.

The frozen samples were thawed and mRNA was extracted using the Invitrogen mRNA Catcher Plus kit (Cat #K1570-02) according to the manufacturer's instructions. Half yield of mRNA from RNA extraction were used to synthesize cDNA in 20 μL reverse transcriptase reactions using Invitrogen SuperScript IV VILO Master Mix (Cat #11756500). TaqMan* real-time PCR was performed using QuantStudio Real-Time PCR system from ThermoFisher (Applied Biosystems). All real-time PCR reactions were run in duplicate using commercial predesigned TaqMan assays for mouse IFIT1, IFIT3, MX1 and PPIA gene expression and TaqMan Master Mix. PPIA was utilized as the housekeeping gene. The recommendations from the manufacturer were followed. All raw data (Ct) were normalized by average housekeeping gene (Ct) and then the comparative Ct (ΔΔCt) method were utilized to quantify relative gene expression (RQ) for experimental analysis.

### Definitions

“Aliphatic” means a straight- or branched-chain, saturated or unsaturated, non-aromatic hydrocarbon moiety having the specified number of carbon atoms (e.g., as in “C₃ aliphatic,” “C₁₋₅ aliphatic,” “C₁-C₅ aliphatic,” or “C₁ to C₅ aliphatic,” the latter three phrases being synonymous for an aliphatic moiety having from 1 to 5 carbon atoms) or, where the number of carbon atoms is not explicitly specified, from 1 to 4 carbon atoms (2 to 4 carbons in the instance of unsaturated aliphatic moieties). A similar understanding is applied to the number of carbons in other types, as in C₂₋₄ alkene, C₄-C₇ cycloaliphatic, etc. In a similar vein, a term such as “(CH₂)₁₋₃” is to be understand as shorthand for the subscript being 1, 2, or 3, so that such term represents CH₂, CH₂CH₂, and CH₂CH₂CH₂.

“Alkyl” means a saturated aliphatic moiety, with the same convention for designating the number of carbon atoms being applicable. By way of illustration, C₁-C₄ alkyl moieties include, but are not limited to, methyl, ethyl, propyl, isopropyl, isobutyl, t-butyl, 1-butyl, 2-butyl, and the like. “Alkanediyl” (sometimes also referred to as “alkylene”) means a divalent counterpart of an alkyl group, such as

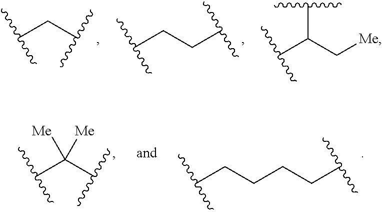

“Alkenyl” means an aliphatic moiety having at least one carbon-carbon double bond, with the same convention for designating the number of carbon atoms being applicable. By way of illustration, C₂-C₄ alkenyl moieties include, but are not limited to, ethenyl (vinyl), 2-propenyl (allyl or prop-2-enyl), cis-1-propenyl, trans-1-propenyl, E- (orZ-) 2-butenyl, 3-butenyl, 1,3-butadienyl (but-1,3-dienyl) and the like.

“Alkynyl” means an aliphatic moiety having at least one carbon-carbon triple bond, with the same convention for designating the number of carbon atoms being applicable. By way of illustration, C₂-C₄ alkynyl groups include ethynyl (acetylenyl), propargyl (prop-2-ynyl), 1-propynyl, but-2-ynyl, and the like.

“Cycloaliphatic” means a saturated or unsaturated, non-aromatic hydrocarbon moiety having from 1 to 3 rings, each ring having from 3 to 8 (preferably from 3 to 6) carbon atoms. “Cycloalkyl” means a cycloaliphatic moiety in which each ring is saturated. “Cyclo-alkenyl”means a cycloaliphatic moiety in which at least one ring has at least one carbon-carbon double bond. “Cycloalkynyl” means a cycloaliphatic moiety in which at least one ring has at least one carbon-carbon triple bond. By way of illustration, cycloaliphatic moieties include, but are not limited to, cyclopropyl, cyclobutyl, cyclopentyl, cyclopentenyl, cyclohexyl, cyclohexenyl, cycloheptyl, cyclooctyl, and adamantyl. Preferred cycloaliphatic moieties are cycloalkyl ones, especially cyclopropyl, cyclobutyl, cyclopentyl, and cyclohexyl. “Cycloalkanediyl” (sometimes also referred to as “cycloalkylene”) means a divalent counterpart of a cycloalkyl group. Similarly, “bicycloalkanediyl” (or “bicycloalkylene”) and “spiroalkanediyl” (or “spiroalkylene”) refer to divalent counterparts of a bicycloalkyl and spiroalkyl (or “spirocycloalkyl”) group.

“Heterocycloaliphatic” means a cycloaliphatic moiety wherein, in at least one ring thereof, up to three (preferably 1 to 2) carbons have been replaced with a heteroatom independently selected from N, O, or S, where the N and S optionally may be oxidized and the N optionally may be quaternized. Preferred cycloaliphatic moieties consist of one ring, 5- to 6-membered in size. Similarly, “heterocycloalkyl,” “heterocycloalkenyl,” and “heterocycloalkynyl” means a cycloalkyl, cycloalkenyl, or cycloalkynyl moiety, respectively, in which at least one ring thereof has been so modified. Exemplary heterocycloaliphatic moieties include aziridinyl, azetidinyl, 1,3-dioxanyl, oxetanyl, tetrahydrofuryl, pyrrolidinyl, piperidinyl, piperazinyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolanyl, tetrahydro-1,1-dioxothienyl, 1,4-dioxanyl, thietanyl, and the like. “Heterocycloalkylene” means a divalent counterpart of a heterocycloalkyl group.

“Alkoxy,” “aryloxy,” “alkylthio,” and “arylthio” mean —O(alkyl), —O(aryl), —S(alkyl), and —S(aryl), respectively. Examples are methoxy, phenoxy, methylthio, and phenylthio, respectively.

“Halogen” or “halo” means fluorine, chlorine, bromine or iodine, unless a narrower meaning is indicated.

“Aryl” means a hydrocarbon moiety having a mono-, bi-, or tricyclic ring system (preferably monocyclic) wherein each ring has from 3 to 7 carbon atoms and at least one ring is aromatic. The rings in the ring system may be fused to each other (as in naphthyl) or bonded to each other (as in biphenyl) and may be fused or bonded to non-aromatic rings (as in indanyl or cyclohexylphenyl). By way of further illustration, aryl moieties include, but are not limited to, phenyl, naphthyl, tetrahydronaphthyl, indanyl, biphenyl, phenanthryl, anthracenyl, and acenaphthyl. “Arylene” means a divalent counterpart of an aryl group, for example 1,2-phenylene, 1,3-phenylene, or 1,4-phenylene.

“Heteroaryl” means a moiety having a mono-, bi-, or tricyclic ring system (preferably 5- to 7-membered monocyclic) wherein each ring has from 3 to 7 carbon atoms and at least one ring is an aromatic ring containing from 1 to 4 heteroatoms independently selected from from N, O, or S, where the N and S optionally may be oxidized and the N optionally may be quaternized. Such at least one heteroatom containing aromatic ring may be fused to other types of rings (as in benzofuranyl or tetrahydroisoquinolyl) or directly bonded to other types of rings (as in phenylpyridyl or 2-cyclopentylpyridyl). By way of further illustration, heteroaryl moieties include pyrrolyl, furanyl, thiophenyl (thienyl), imidazolyl, pyrazolyl, oxazolyl, isoxazolyl, thiazolyl, isothiazolyl, triazolyl, tetrazolyl, pyridyl, N-oxopyridyl, pyridazinyl, pyrimidinyl, pyrazinyl, quinolinyl, isoquinolynyl, quinazolinyl, cinnolinyl, quinozalinyl, naphthyridinyl, benzo-furanyl, indolyl, benzothiophenyl, oxadiazolyl, thiadiazolyl, phenothiazolyl, benzimidazolyl, benzotriazolyl, dibenzofuranyl, carbazolyl, dibenzothiophenyl, acridinyl, and the like. “Heteroarylene” means a divalent counterpart of a heteroaryl group.

Where it is indicated that a moiety may be substituted, such as by use of “unsubstituted or substituted” or “optionally substituted” phrasing as in “unsubstituted or substituted C₁-C₅ alkyl” or “optionally substituted heteroaryl,” such moiety may have one or more independently selected substituents, preferably one to five in number, more preferably one or two in number. Substituents and substitution patterns can be selected by one of ordinary skill in the art, having regard for the moiety to which the substituent is attached, to provide compounds that are chemically stable and that can be synthesized by techniques known in the art as well as the methods set forth herein. Where a moiety is identified as being “unsubstituted or substituted” or “optionally substituted,” in a preferred embodiment such moiety is unsubstituted.

“Arylalkyl,” (heterocycloaliphatic)alkyl,” “arylalkenyl,” “arylalkynyl,” “biarylalkyl,” and the like mean an alkyl, alkenyl, or alkynyl moiety, as the case may be, substituted with an aryl, heterocycloaliphatic, biaryl, etc., moiety, as the case may be, with the open (unsatisfied) valence at the alkyl, alkenyl, or alkynyl moiety, for example as in benzyl, phenethyl, N-imidazoylethyl, N-morpholinoethyl, and the like. Conversely, “alkylaryl,” “alkenylcycloalkyl,” and the like mean an aryl, cycloalkyl, etc., moiety, as the case may be, substituted with an alkyl, alkenyl, etc., moiety, as the case may be, for example as in methylphenyl (tolyl) or allylcyclohexyl. “Hydroxyalkyl,” “haloalkyl,” “alkylaryl,” “cyanoaryl,” and the like mean an alkyl, aryl, etc., moiety, as the case may be, substituted with one or more of the identified substituent (hydroxyl, halo, etc., as the case may be).

For example, permissible substituents include, but are not limited to, alkyl (especially methyl or ethyl), alkenyl (especially allyl), alkynyl, aryl, heteroaryl, cycloaliphatic, heterocycloaliphatic, halo (especially fluoro), haloalkyl (especially trifluoromethyl), hydroxyl, hydroxyalkyl (especially hydroxyethyl), cyano, nitro, alkoxy, —O(hydroxyalkyl), —O(haloalkyl) (especially —OCF₃), —O(cycloalkyl), —O(heterocycloalkyl), —O(aryl), alkylthio, arylthio, ═O, ═NH, ═N(alkyl), ═NOH, ═NO(alkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), —SO₂N(alkyl)₂, and the like.

Where the moiety being substituted is an aliphatic moiety, preferred substituents are aryl, heteroaryl, cycloaliphatic, heterocycloaliphatic, halo, hydroxyl, cyano, nitro, alkoxy, —O(hydroxyalkyl), —O(haloalkyl), —O(cycloalkyl), —O(heterocycloalkyl), —O(aryl), alkylthio, arylthio, ═O, ═NH, ═N(alkyl), ═NOH, ═NO(alkyl), —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(═O)alkyl, —S(cycloalkyl), —SO₂(alkyl), —SO₂NH₂, —SO₂NH(alkyl), and —SO₂N(alkyl)₂. More preferred substituents are halo, hydroxyl, cyano, nitro, alkoxy, —O(aryl), ═O, ═NOH, ═NO(alkyl), —OC(═O)(alkyl), —OC(═O)O(alkyl), —OC(═O)NH₂, —OC(═O)NH(alkyl), —OC(═O)N(alkyl)₂, azido, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are phenyl, cyano, halo, hydroxyl, nitro, C₁-C₄ alkyoxy, O(C₂-C₄ alkanediyl)OH, and O(C₂-C₄ alkanediyl)halo.

Where the moiety being substituted is a cycloaliphatic, heterocycloaliphatic, aryl, or heteroaryl moiety, preferred substituents are alkyl, alkenyl, alkynyl, halo, haloalkyl, hydroxyl, hydroxyalkyl, cyano, nitro, alkoxy, —O(hydroxyalkyl), —O(haloalkyl), —O(aryl), —O(cycloalkyl), —O(heterocycloalkyl), alkylthio, arylthio, —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, azido, —NH₂, —NH(alkyl), —N(alkyl)₂, —NH(aryl), —NH(hydroxyalkyl), —NHC(═O)(alkyl), —NHC(═O)H, —NHC(═O)NH₂, —NHC(═O)NH(alkyl), —NHC(═O)N(alkyl)₂, —NHC(═NH)NH₂, —OSO₂(alkyl), —SH, —S(alkyl), —S(aryl), —S(cycloalkyl), —S(═O)alkyl, —SO₂(alkyl), —SO₂NH₂, —SO₂NH(alkyl), and —SO₂N(alkyl)₂. More preferred substituents are alkyl, alkenyl, halo, haloalkyl, hydroxyl, hydroxyalkyl, cyano, nitro, alkoxy, —O(hydroxyalkyl), —C(═O)(alkyl), —C(═O)H, —CO₂H, —C(═O)NHOH, —C(═O)O(alkyl), —C(═O)O(hydroxyalkyl), —C(═O)NH₂, —C(═O)NH(alkyl), —C(═O)N(alkyl)₂, —OC(═O)(alkyl), —OC(═O)(hydroxyalkyl), —OC(═O)O(alkyl), —OC(═O)O(hydroxyalkyl), —OC(═O)NH₂, —OC(═O)NH(alkyl), —OC(═O)N(alkyl)₂, —NH₂, —NH(alkyl), —N(alkyl)₂, —NH(aryl), —NHC(═O)(alkyl), —NHC(═O)H, —NHC(═O)NH₂, —NHC(═O)NH(alkyl), —NHC(═O)N(alkyl)₂, and —NHC(═NH)NH₂. Especially preferred are C₁-C₄ alkyl, cyano, nitro, halo, and C₁-C₄alkoxy.

Where a range is stated, as in “C₁-C₅ alkyl” or “5 to 10%,” such range includes the end points of the range, as in C₁ and C₅ in the first instance and 5% and 10% in the second instance.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature or symbols), all stereoisomers are included within the scope of the invention, as pure compounds as well as mixtures thereof. Unless otherwise indicated, racemates, individual enantiomers (whether optically pure or partially resolved), diastereomers, geometrical isomers, and combinations and mixtures thereof are all encompassed by this invention.

Those skilled in the art will appreciate that compounds may have tautomeric forms (e.g., keto and enol forms), resonance forms, and zwitterionic forms that are equivalent to those depicted in the structural formulae used herein and that the structural formulae encompass such tautomeric, resonance, or zwitterionic forms.

“Pharmaceutically acceptable ester” means an ester that hydrolyzes in vivo (for example in the human body) to produce the parent compound or a salt thereof or has per se activity similar to that of the parent compound. Suitable esters include C₁-C₅ alkyl, C₂-C₅ alkenyl or C₂-C₅ alkynyl esters, especially methyl, ethyl or n-propyl.

“Pharmaceutically acceptable salt” means a salt of a compound suitable for pharmaceutical formulation. Where a compound has one or more basic groups, the salt can be an acid addition salt, such as a sulfate, hydrobromide, tartrate, mesylate, maleate, citrate, phosphate, acetate, pamoate (embonate), hydroiodide, nitrate, hydrochloride, lactate, methyl-sulfate, fumarate, benzoate, succinate, mesylate, lactobionate, suberate, tosylate, and the like. Where a compound has one or more acidic groups, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenylcyclohexylamine salt, benzathine salt, sodium salt, tetramethylammonium salt, and the like. Polymorphic crystalline forms and solvates are also encompassed within the scope of this invention.

“Subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In the formulae of this specification, a wavy line ( [symbol] ) transverse to a bond or an asterisk (*) at the end of the bond denotes a covalent attachment site. For instance, a statement that R is

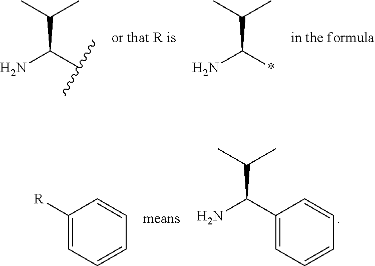

In the formulae of this specification, a bond traversing an aromatic ring between two carbons thereof means that the group attached to the bond may be located at any of the positions of the aromatic ring made available by removal of the hydrogen that is implicitly there (or explicitly there, if drawn out). By way of illustration, the formula

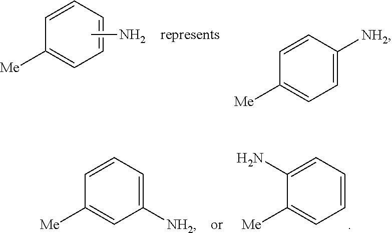

In other illustrations,

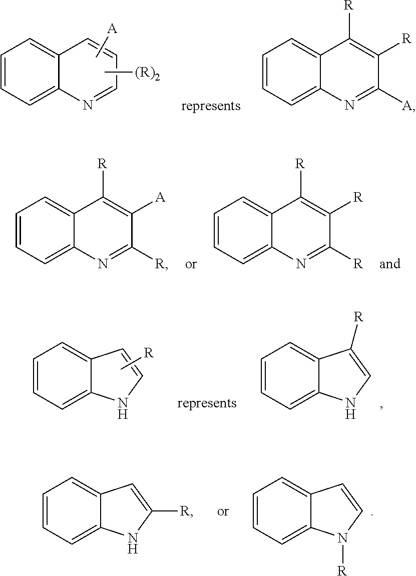

This disclosure includes all isotopes of atoms occurring in the compounds described herein. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. By way of example, a C₁-C₃ alkyl group can be undeuterated, partially deuterated, or fully deuterated and “CH₃” includes CH₃, ¹³CH₃, ¹⁴CH₃, CH₂T, CH₂D, CHD₂, CD₃, etc. In one embodiment, the various elements in a compound are present in their natural isotopic abundance.

Those skilled in the art will appreciate that certain structures can be drawn in one tautomeric form or another—for example, keto versus enol—and that the two forms are equivalent.

### Acronyms and Abbreviations

This is a list of acronyms and abbreviations used in this specification, along with their meanings.

| ACRONYM OR | |
|---|---|
| ABBREVIATION | MEANING OR DEFINITION |
| AIBN | Azobisisobutyronitrile |
| Alloc | Allyloxycarbonyl |
| Aq. | Aqueous |
| Boc | t-Butyloxycarbonyl |
| BOP | (Benzotriazol-1-yloxy)tris(dimethylamino)- |
| | phosphonium hexafluorophosphate (V) |
| BOP | (Benzotriazol-1-yloxy)tris(dimethylamino)- |
| | phosphonium hexafluorophosphate (V) |
| DBU | 1,8-Diazabicyclo[5.4.0]undec-7-ene |
| DCM | Dichloromethane |
| DIAD | Diisopropyl azodicarboxylate |
| DIPEA, DIEA | N,N-diisopropylethylamine, also known as |
| | Hünig′s base |
| DMA | N,N-Dimethylacetamide |
| DMAP | 4-(Dimethylamino)pyridine |
| DMF | N,N-dimethylformamide |
| DMSO | Dimethyl sulfoxide |
| DTDP | 2,2′-dithiodipyridine |
| DTPA | Diethylenetriaminepentaacetic acid |
| EEDQ | Ethyl 2-ethoxyquinoline-1(2H)-carboxylate |
| Fmoc | Fluorenylmethyloxycarbonyl |
| HATU | Hexafluorophosphate Azabenzotriazole |
| | Tetramethyl Uronium; 1-[Bis(dimethylamino)- |
| | methylene]-1H-1,2,3-triazolo[4,5-b] |
| | pyridinium 3-oxide hexafluorophosphate |
| HEPES | 4-(2-Hydroxyethyl)piperazine-1-ethanesulfonic |
| | acid, N-(2-Hydroxyethyl)piperazine-N′-(2- |
| | ethanesulfonic acid) |
| HPLC | High pressure liquid chromatography |
| Hunig′s base | See DIPEA, DIEA |
| LCMS, LC-MS, LC/MS | Liquid chromatography-mass spectrometry |
| mCPBA | m-chloroperbenzoic acid |
| MS | Mass spectrometry |
| MsCl | Methanesylfonyl chloride, mesyl chloride |
| NBS | N-Bromosuccinimide |
| NMR | Nuclear magnetic resonance |
| PEG | Poly(ethylene glycol) |
| PTFE | Poly(tetrafluoroethylene) |
| RT (in context of liquid | Retention time, in min |
| chromatography) | |
| RT (in the context of | Room (ambient) temperature, circa 25° C. |
| reaction conditions) | |
| Sat. | Saturated |
| Soln | Solution |
| TBDPS | tert-Butyldiphenylsilyl |
| TBS | t-Butyldimethylsily group |
| TEA | Triethylamine |
| TEAA | Triethylammonium acetate |
| TFA | Trifluoroacetic acid |
| THF | Tetra hydrofuran |

### REFERENCES

Full citations for the following references cited in abbreviated fashion by first author (or inventor) and date earlier in this specification are provided below. Each of these references is incorporated herein by reference for all purposes.
Akinbobuyi et al., Tetrahedron Lett. 2015, 56, 458, “Facile syntheses of functionalized toll-like receptor 7 agonists”.
Akinbobuyi et al., Bioorg. Med. Chem. Lett. 2016, 26, 4246, “Synthesis and immunostimulatory activity of substituted TLR7 agonists.”
Barberis et al., US 2012/0003298 A1 (2012).
Beesu et al., J. Med. Chem. 2017, 60, 2084, “Identification of High-Potency Human TLR8 and Dual TLR7/TLR8 Agonists in Pyrimidine-2,4-diamines.”
Berghöfer et al., J. Immunol. 2007, 178, 4072, “Natural and Synthetic TLR7 Ligands Inhibit CpG-A- and CpG-C-Oligodeoxynucleotide-Induced IFN-α Production.”
Bonfanti et al., US 2014/0323441 A1 (2015) [2015a].
Bonfanti et al., US 2015/0299221 A1 (2015) [2015b].
Bonfanti et al., US 2016/0304531 A1 (2016).
Carson et al., US 2013/0202629 A1 (2013).
Carson et al., U.S. Pat. No. 8,729,088 B2 (2014).
Carson et al., U.S. Pat. No. 9,050,376 B2 (2015).
Carson et al., US 2016/0199499 A1 (2016).
Chan et al., Bioconjugate Chem. 2009, 20, 1194, “Synthesis and Immunological Characterization of Toll-Like Receptor 7 Agonistic Conjugates.”
Chan et al., Bioconjugate Chem. 2011, 22, 445, “Synthesis and Characterization of PEGylated Toll Like Receptor 7 Ligands.”
Chen et al., U.S. Pat. No. 7,919,498 B2 (2011).
Coe et al., U.S. Pat. No. 9,662,336 B2 (2017).
Cortez and Va, Medicinal Chem. Rev. 2018, 53, 481, “Recent Advances in Small-Molecule TLR7 Agonists for Drug Discovery”.
Cortez et al., US 2017/0121421 A1 (2017).
Cortez et al., U.S. Pat. No. 9,944,649 B2 (2018).
Dellaria et al., WO 2007/028129 A1 (2007).
Desai et al., U.S. Pat. No. 9,127,006 B2 (2015).
Ding et al., WO 2016/107536 A1 (2016).
Ding et al., US 2017/0273983 A1 (2017) [2017a].
Ding et al., WO 2017/076346 A1 (2017) [2017b].
Gadd et al., Bioconjugate Chem. 2015, 26, 1743, “Targeted Activation of Toll-Like Receptors: Conjugation of a Toll-Like Receptor 7 Agonist to a Monoclonal Antibody Maintains Antigen Binding and Specificity.”
Graupe et al., U.S. Pat. No. 8,993,755 B2 (2015).
Embrechts et al., J. Med. Chem. 2018, 61, 6236, “2,4-Diaminoquinazolines as Dual Toll Like Receptor (TLR) 7/8 Modulators for the Treatment of Hepatitis B Virus.”
Halcomb et al., U.S. Pat. No. 9,161,934 B2 (2015).
Hashimoto et al., US 2009/0118263 A1 (2009).
He et al., U.S. Pat. No. 10,487,084 B2 (2019) [2019a].
He et al., U.S. Pat. No. 10,508,115 B2 (2019) [2019b].
Hirota et al., U.S. Pat. No. 6,028,076 (2000).
Holldack et al., US 2012/0083473 A1 (2012).
Isobe et al., U.S. Pat. No. 6,376,501 B1 (2002).
Isobe et al., JP 2004137157 (2004).
Isobe et al., J. Med. Chem. 2006, 49 (6), 2088, “Synthesis and Biological Evaluation of Novel 9-Substituted-8-Hydroxyadenine Derivatives as Potent Interferon Inducers.”
Isobe et al., U.S. Pat. No. 7,521,454 B2 (2009) [2009a].
Isobe et al., US 2009/0105212 A1 (2009) [2009b].
Isobe et al., US 2011/0028715 A1 (2011).
Isobe et al., U.S. Pat. No. 8,148,371 B2 (2012).
Jensen et al., WO 2015/036044 A1 (2015).
Jones et al., U.S. Pat. No. 7,691,877 B2 (2010).
Jones et al., US 2012/0302598 A1 (2012).
Kasibhatla et al., U.S. Pat. No. 7,241,890 B2 (2007).
Koga-Yamakawa et al., Int. J. Cancer 2013, 132 (3), 580, “Intratracheal and oral administration of SM-276001: A selective TLR7 agonist, leads to antitumor efficacy in primary and metastatic models of cancer.”
Li et al., U.S. Pat. No. 9,902,730 B2 (2018).
Lioux et al., U.S. Pat. No. 9,295,732 B2 (2016).
Lund et al., Proc. Nat'l Acad. Sci (USA) 2004, 101 (15), 5598, “Recognition of single-stranded RNA viruses by Toll-like receptor 7.”
Maj et al., U.S. Pat. No. 9,173,935 B2 (2015).
McGowan et al., US 2016/0168150 A1 (2016) [2016a].
McGowan et al., U.S. Pat. No. 9,499,549 B2 (2016) [2016b].
McGowan et al., J. Med. Chem. 2017, 60, 6137, “Identification and Optimization of Pyrrolo[3,2-d]pyrimidine Toll-like Receptor 7 (TLR7) Selective Agonists for the Treatment of Hepatitis B.”
Musmuca et al., J. Chem. Information & Modeling 2009, 49 (7), 1777, “Small-Molecule Interferon Inducers. Toward the Comprehension of the Molecular Determinants through Ligand-Based Approaches.”
Nakamura et al., Bioorg. Med. Chem. Lett. 2013, 13, 669, “Synthesis and evaluation of 8-oxoadenine derivatives as potent Toll-like receptor agonists with high water solubility.”
Ogita et al., US 2007/0225303 A1 (2007).
Ota et al., WO 2019/124500 A1 (2019).
Pilatte et al., WO 2017/216293 A1 (2017).
Poudel et al., U.S. Pat. No. 10,472,361 B2 (2019) [2019a].
Poudel et al., U.S. Pat. No. 10,494,370 B2 (2019) [2019b].
Poudel et al., US 2020/0038403 A1 (2020) [2020a].
Poudel et al., US 2020/0039986 A1 2020) [2020b].
Purandare et al., WO 2019/209811 A1 (2019).
Pryde, U.S. Pat. No. 7,642,350 B2 (2010).
Sato-Kaneko et al., JCI Insight 2017, 2, e93397, “Combination Immunotherapy with TLR Agonists and Checkpoint Inhibitors Suppresses Head and Neck Cancer”.
Smits et al., The Oncologist 2008, 13, 859, “The Use of TLR7 and TLR8 Ligands for the Enhancement of Cancer Immunotherapy”.
Vasilakos and Tomai, Expert Rev. Vaccines 2013, 12, 809, “The Use of Toll-like Receptor 7/8 Agonists as Vaccine Adjuvants”.
Vernejoul et al., US 2014/0141033 A1 (2014).
Young et al., U.S. Pat. No. 10,457,681 B2 (2019).
Yu et al., PLoS One 2013, 8 (3), e56514, “Toll-Like Receptor 7 Agonists: Chemical Feature Based Pharmacophore Identification and Molecular Docking Studies.”
Zhang et al., Immunity 2016, 45, 737, “Structural Analysis Reveals that Toll-like Receptor 7 Is a Dual Receptor for Guanosine and Single-Stranded RNA.”
Zhang et al., WO 2018/095426 A1 (2018)>
Zurawski et al., US 2012/0231023 A1 (2012).

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A compound having a structure according to formula I 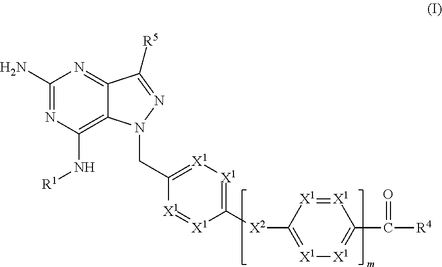
wherein
each X¹ is independently N or CR²;
X² is O, CH₂, NH, S, or N(C₁-C₃ alkyl);
R¹ is (C₁-C₅ alkyl),
(C₂-C₅ alkenyl),
(C₁-C₈ alkanediyl)₀₋₁(C₃-C₆ cycloalkyl),
(C₂-C₈ alkanediyl)OH,
(C₂-C₈ alkanediyl)O(C₁-C₃ alkyl),
(C₁-C₄ alkanediyl)₀₋₁(5-6 membered heteroaryl),
(C₁-C₄ alkanediyl)₀₋₁phenyl,
(C₁-C₄ alkanediyl)CF₃,
(C₂-C₈ alkanediyl)N[C(═O)](C₁-C₃ alkyl),
or
(C₂-C₈ alkanediyl)NR^{x}R^{y};
each R² is independently H, O(C₁-C₃ alkyl), S(C₁-C₃ alkyl), SO₂(C₁-C₃ alkyl), C₁-C₃ alkyl, O(C₃-C₄ cycloalkyl), S(C₃-C₄ cycloalkyl), SO₂(C₃-C₄ cycloalkyl), C₃-C₄ cycloalkyl, Cl, F, CN, or [C(═O)]₀₋₁NR^{x}R^{y};
R⁴ is NH₂,
NH(C₁-C₅ alkyl),
N(C₁-C₅ alkyl)₂,
NH(C₁-C₄ alkanediyl)₀₋₁(C₃-C₈ cycloalkyl),
N(C₃-C₆ cycloalkyl)₂,
or
a moiety having the structure 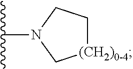
R⁵ is H, C₁-C₅ alkyl, C₂-C₅ alkenyl, C₃-C₆ cycloalkyl, halo, O(C₁-C₅ alkyl), (C₁-C₄ alkanediyl)OH, (C₁-C₄ alkanediyl)O(C₁-C₃ alkyl), phenyl, NH(C₁-C₅ alkyl), 5 or 6 membered heteroaryl, 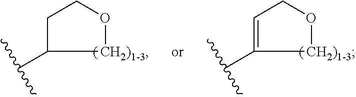
R^{x} and R^{y} are independently H or C₁-C₃ alkyl or R^{x} and R^{y} combine with the nitrogen to which they are bonded to form a 3- to 7-membered heterocycle;
and
m is 0 or 1;
wherein in R¹, R², R⁴, and R⁵
an alkyl, alkanediyl, cycloalkyl, phenyl, 5 or 6-membered heteroaryl, or moiety of the formula 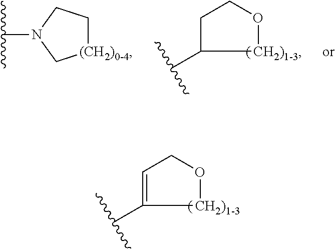
is optionally substituted with one or more substituents selected from OH, halo, CN, (C₁-C₃ alkyl), O(C₁-C₃ alkyl), C(═O)(C₁-C₃ alkyl), SO₂(C₁-C₃ alkyl), NR^{x}R^{y}, (C₁-C₄ alkanediyl)OH, (C₁-C₄ alkanediyl)O(C₁-C₃ alkyl); and
an alkyl, alkanediyl, cycloalkyl, or cyclic moiety of the formula 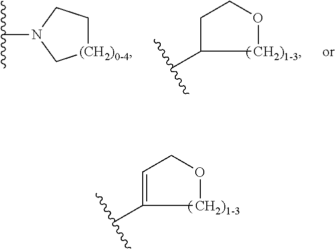
may have a CH₂ group replaced by O, S₂, CF₂, C(═O), NH,
N[C(═O)]₀₋₁(C₁-C₃ alkyl),
N[C(═O)]₀₋₁(C₁-C₄ alkanediyl)CF₃,
N[C(═O)]₀₋₁(C₁-C₄ alkanediyl)OH,
or
N[C(═O)]₀₋₁(C₁-C₄ alkanediyl)₀₋₁(C₃-C₅ cycloalkyl).

2. A compound according to claim 1, wherein, in formula (I), the moiety

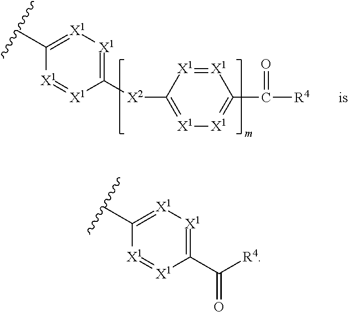

3. A compound according to claim 1, having a structure according to formula (Ia):

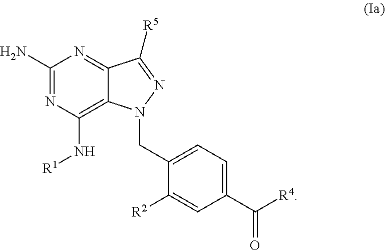

4. A compound according to claim 3, wherein R¹ is selected from the group consisting of

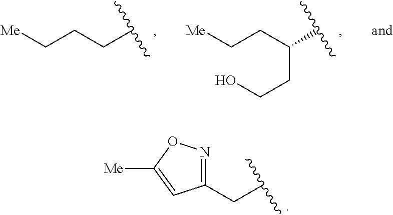 [SMILES: *CCCC ; *[C@@H](CCC)CCO ; *Cc1cc(C)on1]

5. A compound according to claim 3, wherein R² is

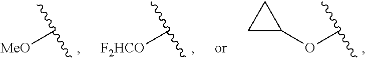

6. A compound according to claim 3, wherein R⁵ is H or Me.

7. A compound according to claim 3, wherein R⁴ is

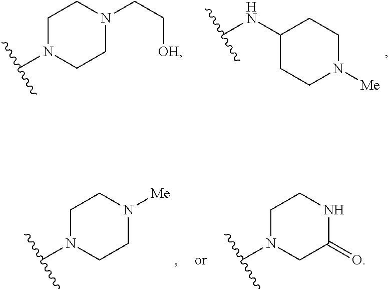 [SMILES: *N1CCN(CCO)CC1 ; *NC1CCN(C)CC1 ; *N1CCN(C)CC1 ; *N1CCNC(=O)C1]

8. A compound according to claim 7, wherein R¹ is selected from the group consisting of

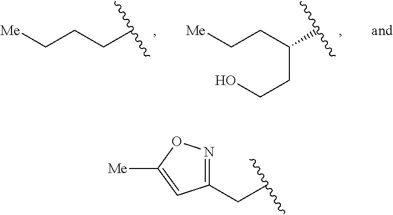 [SMILES: *CCCC ; *[C@@H](CCC)CCO ; *Cc1cc(C)on1]

9. A compound according to claim 1, having a structure according to formula (Ib):

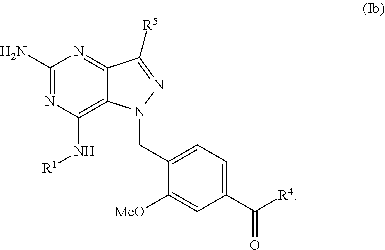

10. A compound according to claim 9
wherein
R¹ is 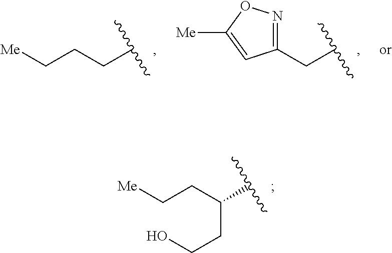 [SMILES: *CCCC ; *Cc1cc(C)on1 ; *[C@@H](CCC)CCO]
R⁴ is 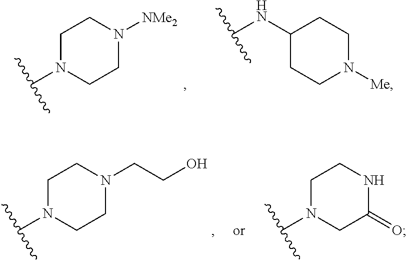 [SMILES: *N1CCN(N(C)C)CC1 ; *NC1CCN(C)CC1 ; *N1CCN(CCO)CC1 ; *N1CCNC(=O)C1]
and
R⁵ is H, Me, or F.

11. A method of treating a cancer, comprising administering to a patient suffering from such cancer a therapeutically effective combination of an anti-cancer immunotherapy agent and a compound according to claim 1.

12. A method according to claim 11, wherein the anti-cancer immunotherapy agent is an antagonistic anti-CTLA-4, anti-PD-1, or anti-PD-L1 antibody.

13. A method according to claim 11, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

14. A method according to claim 13, wherein the anti-cancer immunotherapy agent is ipilimumab, nivolumab, or pembrolizumab.

15. A method of treating a cancer, comprising administering to a patient suffering from such cancer a therapeutically effective combination of an anti-cancer immunotherapy agent and a compound according to claim 10.

16. A method according to claim 15, wherein the anti-cancer immunotherapy agent is an antagonistic anti-CTLA-4, anti-PD-1, or anti-PD-L1 antibody.

17. A method according to claim 15, wherein the cancer is lung cancer (including non-small cell lung cancer), pancreatic cancer, kidney cancer, head and neck cancer, lymphoma (including Hodgkin's lymphoma), skin cancer (including melanoma and Merkel skin cancer), urothelial cancer (including bladder cancer), gastric cancer, hepatocellular cancer, or colorectal cancer.

18. A method according to claim 17, wherein the anti-cancer immunotherapy agent is ipilimumab, nivolumab, or pembrolizumab.
